SEQUENCE LISTING (3 sequences)

SEQ ID NO: 1 - protein, 465 aa, Chromobacterium violaceum
HHHHHHMQKQRTTSQWRELDAAHHLHPFTDTASLNQAGARVMTRGEGVYLWDSEGNKIIDGMAGLCCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 2 - protein, 466 aa, Vibrio fluvialis
MASRGSHHHHHHGANKPQSWEARAETYSLYGFTDMPSLHQRGTVVVTHGEGPYIVDVNGRRYLDANSGLWNMVAGFDHKGLIDAAKAQYERFPGYHAFFGRMSDQTVMLSEKLVEVSPFDSGRVFYTNSGSEANDTMVKMLWFLHAAEGKPQKRKILTRWNAYHGVTAVSASMTGKPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPMGAVILGPELSKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICRPLGQSVVLCPPFILTEAQMDEMFDKLEKALDKVFAEVA

SEQ ID NO: 3 - protein, 453 aa, Vibrio fluvialis
MNKPQSWETRAETYSLYGFTDMPSLHQRGTVVVTHGEGPYIVDVHGRRYLDANSGALNMVAGFDHKGLIDAAKTQYERFPGYHAFSGRMSDQTVMLSEKLVEVSPFDSGRVFYTNSGSEANDTMVKMLWFLHAAEGKPQKRKILTRWNAYHGATAVSASMTGFPYNSVFGLPLPGFLHLTCPHYWRYGEEGETEEQFVARLARELEETIQKEGADTIAGFFAEPVMGAGGVIPPAKGYFQAILPILRKYDIPVISDEVICGFGRTGNTWGCVTYDFTPDAIISSKSLTAGFFPVGAVILGPELSKRLETAIEAIEEFPHGFTTGGHPVGCAIALKAIDVVMNEGLAENVRRLAPRFEERLKHIAERPNIGEYRGIGFMWALEVVKDKASKAPFGGNLSVSERIANTCTDLGLICRPLGQSVVLCPPFILTEAQMDEMFDKLEKALDKVFAEVA

(19) United States
(12) Patent Application Publication
(10) Pub. No.: US 2025/0122542 A1
(43) Pub. Date: Apr. 17, 2025

(54) PROCESS TO PRODUCE (1R,4R)-4-SUBSTITUTED CYCLOHEXANE-1-AMINES

(71) Applicants: Richter Gedeon Nyrt., Budapest (HU); BUDAPESTI MÜSZAKI ÉS GAZDASÁGTUDOMÁNYI EGYETEM, Budapest (HU)

(72) Inventors: Emese Farkas, Aszalo (HU); László Poppe, Budapest (HU); Gábor Hornyánszky, Budapest (HU); Dániel János Incze, Budapest (HU); János Éles, Budapest (HU); Evelin Sánta-Bell, Budapest (HU); Zsófia Klára Molnár, Budapest (HU); József Szemes, Szigethalom (HU); Anna Schneider, Zalaegerszeg (HU); Pál Csuka, Pilisvorosvar (HU)

(21) Appl. No.: 18/689,627

(22) PCT Filed: Sep. 14, 2022
(86) PCT No.: PCT/IB2022/058641

§ 371 Date: Mar. 6, 2024

(30) Foreign Application Priority Data
Sep. 15, 2021 (HU) P2100325
Sep. 13, 2022 (HU) P2200363

(51) Int. Cl. C12P 13/00; C12P 17/04; C12P 41/00

(52) U.S. Cl. CPC C12P 13/001; C12P 17/04; C12P 41/006

(57) ABSTRACT

The invention relates to a process to produce a (1r,4r)-4-substituted cyclohexane-1-amine [further referred as trans-4-substituted cyclohexane-1-amine] of formula (T), starting from a diastereomeric mixture of 4-substituted cyclohexane-1-amines (formula (C)+formula (T)) or any salt of them by using a single transaminase biocatalyst in whole-cell, soluble or immobilized form in the presence of an amine acceptor used in sub-equimolar up to equimolar quantities in batch mode or in continuous-flow mode. In the first aspect of the present invention 2-(trans-4-aminocyclohexyl)acetic acid esters, more preferably a C₁₋₆ alkyl esters, particularly 2-(trans-4-aminocyclohexyl)acetic acid ethyl ester may be produced. In the second aspect of the present invention hydroxyl-protected or protective group-free trans-4-(2-hydroxyethyl)cyclohexan-1-amines, particularly trans-4-(2-hydroxyethyl)cyclohexan-1-amine may be produced. In the third aspect of the present invention protected 2-(trans-4-aminocyclohexyl)acetaldehydes, particularly trans-4-((1,3-dioxolan-2-yl)methyl)cyclohexan-1-amine may be produced.

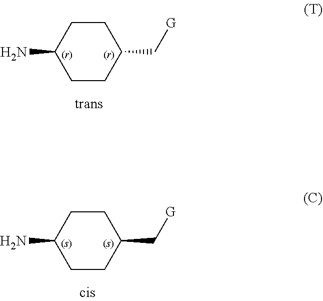

### FIELD OF THE INVENTION

The present invention relates to a new process to produce a (1r,4r)-4-substituted cyclohexane-1-amine [further referred as trans-4-substituted cyclohexane-1-amine] of formula (T), starting from a diastereomeric mixture of 4-substituted cyclohexane-1-amines (formula (C)+formula (T))

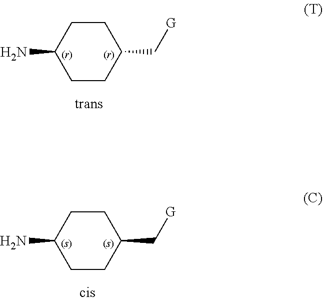

or any salt of them by using a single transaminase biocatalyst in whole-cell, soluble or immobilized form in the presence of an amine acceptor used in sub-equimolar up to equimolar quantities in batch mode or in continuous-flow mode. In the first aspect of the present invention 2-(trans-4-aminocyclohexyl)acetic acid esters, more preferably a C₁-6 alkyl esters, particularly 2-(trans-4-aminocyclohexyl)acetic acid ethyl ester may be produced. In the second aspect of the present invention hydroxyl-protected or protective group-free trans-4-(2-hydroxyethyl)cyclohexan-1-amines, particularly trans-4-(2-hydroxyethyl)cyclohexan-1-amine may be produced. In the third aspect of the present invention protected 2-(trans-4-aminocyclohexyl)acetaldehydes, particularly trans-4-((1,3-dioxolan-2-yl)methyl)cyclohexan-1-amine may be produced.

### TECHNICAL FIELD

The 2-(trans-4-aminocyclohexyl)acetic acid esters, preferably C₁-6 alkyl esters, particularly trans-4-amino-cyclohexyl acetic acid ethyl ester are excellent starting materials for the synthesis of active pharmacological agents, including 3-((1r,4r)-4-(2-(4-(2,3-dichloro-phenyl)piperazin-1-yl)ethyl)cyclohexyl)-1,1-dimethylurea [referred as: trans-N-{4-[2-[4-(2,3-dichlorophenyl)-piperazin-1-yl]ethyl]cyclohexyl}-N′,N′-dimethylurea], commonly known as Cariprazine, the synthesis of which was first disclosed in the international patent application WO 2005/012266A1.

Cariprazine, marketed as Reagila® in Europe and as Vraylar® in the USA, is an atypical antipsychotic (E. Agai-Csongor et al. Bioorg. Med. Chem. Lett. 22, 3437-3440 (2012), DOT: 10.1016/j.bmcl.2012.03.104) for the treatment of schizophrenia and bipolar mania/mixed episodes (L. Citrome, Expert Opin. Drug Metab. Toxicol. 9, 193-206 (2013), DOI: 10.1517/17425255.2013.759211). It is a dopamine receptor partial agonist (D3 and D2) with high selectivity for the D3 receptor (D3 antagonist) in the central nervous system.

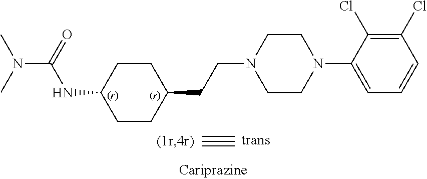

A trans-4-substituted cyclohexaneamine unit (containing two centers of pseudoasymmetry, 1r and 4r providing the trans arrangement) is a crucial element of the chemical structure of the active pharmaceutical agent Cariprazine. Accordingly, for the synthesis of Cariprazine only the diastereomerically pure trans-isomer form (I) of the intermediate compound is applicable and the presence of the diastereomeric cis-isomer form (II) is undesired.

| 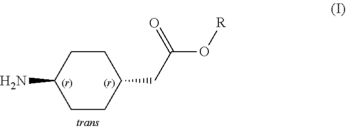 | |
|---|---|
| 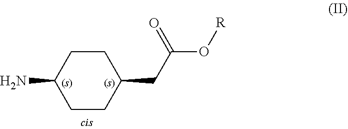 | |
| 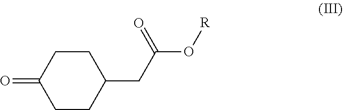 | |
| (I-III) | R |
| a | Me |
| b | Et |
| c | Pr |
| d | iPr |

The stereochemistry within the amino ester of formula (I) is indeed trans (meaning that the 1- and 4-substituents are on two opposite sides of the “unfolded” cyclohexane ring), but when broken down into basic stereogenic elements it is actually caused by the presence of two centers of pseudoasymmetry in the cyclohexane ring (1r,4r) (the centers of pseudoasymmetry are marked r/s in the CIP system; for more details, see L. Poppe et al. Stereochemistry and Stereoselective Synthesis, Wiley-VCH Verlag KGaA, Weinheim (2016), pp. 52-54). A peculiar feature of this system is that if one center of pseudoasymmetry is eliminated (the 1r center is destroyed upon deamination to ketone (III)), the other (in this case 4r) is also eradicated without altering any of the four covalent bonds directly attached to the central atom. In this respect, the system caused by the presence of two centers of pseudoasymmetry behaves rather as a single stereogenic unit, therefore the explanation with one stereodescriptor (cis or trans) is more appropriate.

The (1r,4r)-4-(2-ethoxy-2-oxoethyl)cyclohexan-1-aminium chloride [further referred as 2-(trans-4-aminocyclohexyl)acetic acid ethyl ester hydrochloride] starting material, characterized with formula (Ib·HCl), is provided in industrial scale via simple reaction steps in high quality in accordance with the production process described in WO2010/070368.

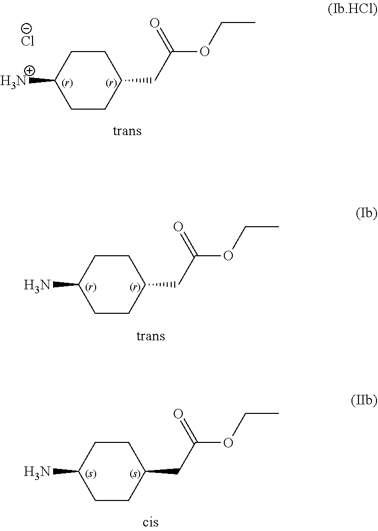

In the process, 2-(4-nitrophenyl)acetic acid, a cheap and readily available large-scale industrial chemical raw material, is hydrogenated in an aqueous medium using a Pd/C catalyst. In the first step, the nitro group and then the aromatic ring are saturated, then the diastereomeric mixture of 2-(4-aminocyclohexyl)acetic acid is esterified in an ethanol medium in an acid-catalyzed reaction, and from the obtained 2-(4-aminocyclohexyl)acetic acid ethyl ester isomeric mixture (Ib+IIb), pure 2-(trans-4-aminocyclohexyl)acetic acid ethyl ester hydrochloride (Ib·HCl) final product is obtained by selective crystallization.

By using this procedure cyclohexyl acetic esters (I+II), for example ethyl (Ib+IIb) and propyl ester (Ic+IIc) derivatives, are prepared in a mixture of trans/cis-isomers in a ratio of about 1:1 and that can be separated to trans- and cis-products (compounds I and II, respectively) with a relatively good yield of the trans-diastereomer (I).

According to the description of WO2010/070368, 2-(trans-4-aminocyclohexyl)acetic acid ethyl ester hydrochloride (Ib·HCl) can be isolated in 40% yield with extremely high diastereomeric purity after acetonitrile treatment. Usually, the trans/cis mixture (Ib+IIb) is separated by crystallization.

According to the publications of X.-W. Chen et al. (Synthesis 48(18), 3120-3126 (2016), DOI: 10.1055/s-0035-1561865; Eur. J. Med. Chem. 123, 332-353 (2016), DOI: 10.1016/j.ejmech.2016.07.038) the N-acylated (N-acetyl, N-Boc, N—(N′-dimethylamino)carbonyl) derivatives of 2-(trans-4-aminocyclohexyl)acetic acid ethyl ester hydrochloride (Ib·HCl) can be isolated by direct recrystallization in ethanol from the cis/trans isomer mixture with moderate isolated yield (64% and 62% for the N-acetyl and N—(N′-dimethylamino)carbonyl derivatives, respectively) and excellent diastereomeric purity (trans:cis˜100:0).

According to the description of CN2015/10649822, 4-aminocyclohexanone without protection of the amino moiety is adopted as raw material. From this compound, Witting reaction is conducted with (dimethoxyphosphinyl)acetic acid methyl ester or with (diethoxyphosphinyl)acetic acid ethyl ester, and then through catalytic hydrogenation of the resulted crude alkyl 2-(trans/cis-4-aminocyclohexylidene)acetate (Ia+IIa, or Ib+IIb) is subjected to salifying crystallization to obtain the purer trans product (compound Ia or Ib, with trans:cis ratio of 95-99.9:5-0.1).

Usually, after a catalytic reduction the trans and cis mixture of the corresponding 2-(4-aminocyclohexyl)acetic acid ester (I+II) is separated by crystallization and the cis by-product (II) is discarded. Isomerization of the cis by-product (II) to a mixture of cis- and trans-isomer is also feasible according to the process of R. S. Muromova, et el. (Zhurnal Vsesoyuznogo Khimicheskogo Obshchestva im. D. I. Mendeleeva, 10(6), 711-712 (1965): Chem. Abstr. 64, 51662 (1966); USSR, SU177422 1965-12-18: Chem. Abstr. 64, 103634 (1966)). In this process, ethyl 2-(4-nitrophenyl)acetate was hydrogenated over Rh—Pt at 60° C. and 100 atm in EtOH to 98% mixed cis/trans-isomers of 2-(4-aminocyclohexyl)acetic acid ethyl ester (compounds Ib+IIb), which were hydrolyzed to the mixed isomers of the free acid. Treatment of either isomers (compound Ib or compound IIb) or isomeric mixture of 2-(4-aminocyclohexyl)acetic acid ethyl ester (compounds Ib+IIb) with 29% H₂O₂ in aqueous NaOH in the presence of tungstic acid gave 80% of the ethyl 4-oxocyclohexylacetate oxime, which in 12 h at 100° C. in aqueous NaOH gave the corresponding isomeric composition of free acid oxime. Electrolytic reduction of the free acid oxime in EtOH-10% H₂SO₄ at 10-12° C. and 3 A dm⁻² gave the mixed 2-(cis/trans-4-aminocyclohexyl)acetic acid, containing 50% of trans-isomer.

According to the description of CN2018/10649822 (Chem. Abstr. 172, 252756 (2019)), Cariprazin can be prepared also from trans-4-(2-hydroxyethyl)cyclohexan-1-aminium chloride (IVa·HCl)

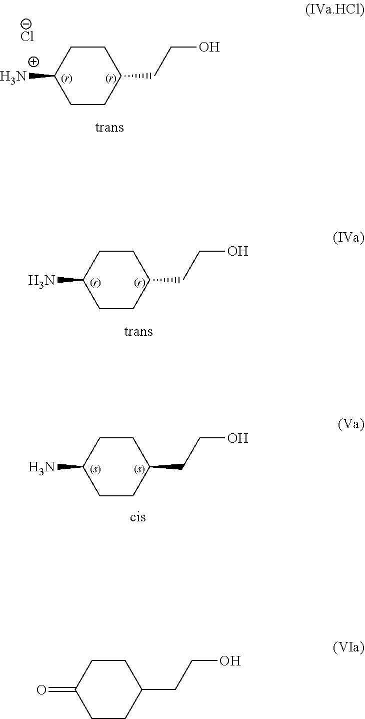 [SMILES: [Cl-] ; [NH3+][C@H]1CC[C@H](CCO)CC1 ; N[C@H]1CC[C@H](CCO)CC1 ; N[C@H]1CC[C@@H](CCO)CC1 ; O=C1CCC(CCO)CC1]

This compound, however, can be prepared from 2-(4-nitrophenyl)ethan-1-ol by hydrogenation in autoclave under high pressure by a costly ruthenium catalyst providing the cis/trans-diastereomeric mixture of 4-(2-hydroxyethyl)cyclohexan-1-amines (compound IVa+compound Va) followed by crystallization-aided isomer separation. In case of these diastereomers (compound IVa+compound Va), diminishing one center of pseudoasymmetry (e.g. the 1r center is not present in ketone (VIa)) also eradicates the other (4r).

According to the description of WO2002006229 (Chem. Abstr. 136, 134675 (2002)), heterocyclic amino alcohol β-3 adrenergic receptor agonists resembling to the structure of Cariprazine can be prepared from trans-4-((1,3-dioxolan-2-yl)methyl)cyclohexan-1-aminium chloride (VIIa·HCl).

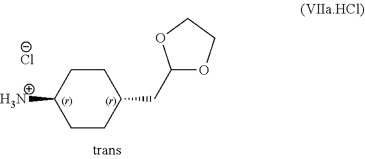 [SMILES: [NH3+][C@H]1CC[C@H](CC2OCCO2)CC1 ; [Cl-]]

The process involves first conversion of the amine moiety of the free base form of the trans-amine (compound VIIa) to an N′,N′-dimethylurea derivative, followed by liberating the aldehyde moiety and coupling with the proper secondary amine reagent.

Our aim was to improve the currently most common manufacturing process in which the cis-isomer (formula II) was treated as waste until now, and to make it possible to convert a predominantly large proportion of the cis by-product (formula II) into the desired trans-isomer (formula I) in a single step under mild conditions.

Initially, we have assumed that separation of cis- and trans-isomers of 2-(4-aminocyclohexyl)acetic acid esters (compounds I+II) can be carried out much more effectively by biocatalytic processes than in the case of separation processes that have been part of the traditional production method to date. Surprisingly, we have fund that a single enzyme can catalyze not only separation of the cis-diastereomer (compound II) but a dynamic isomerization process for the cis-diastereomer of 2-(4-aminocyclohexyl)acetic acid esters (compound II) to the desired trans-intermediate compound (I).

With the explosive development of bioinformatics, enzymology, and molecular biology, practical applications of biocatalytic processes can be well designed and become competitive processes nowadays.

Although for similar related compounds, ketoreductase (reduction of 4-propylcyclohexanone and 4-ethylcyclohexanone to the corresponding cis- and trans-alcohol, respectively) and transaminase [methyl 2-(3-oxocyclohexyl)acetate derivatives and 2-methylcyclohexanone to the corresponding amines] catalyzed investigations are available in the literature, no biocatalytic processes have been developed for the stereoselective preparation of any of the diastereomers of 2-(4-aminocyclohexyl)acetic acid esters (I or II) to date.

It has been hypothesized that a transaminase (TA) catalyzed process for an alkyl 2-(trans-4-aminocyclohexyl)acetate (compound I), especially for ethyl 2-(trans-4-aminocyclohexyl)-acetate hydrochloride described by the chemical structure (Ib·HCl) may be effective in achieving this goal.

In addition, it seemed to be possible that the process could be carried out not only in batch mode but also in a continuous flow mode. Many examples of immobilization and continuous-flow application of transaminases can be found in the literature which can enhance stability, activity and efficiency of enzymes and enable their recovery and use of them in a green and intensified processes. Enzymes can be used as biocatalysts in isolated (purified) form, in the extract of the host cell that produces them (either wild or recombinant), or even embedded in the whole cell.

Transaminases (TAs) catalyze the redox-neutral amino group transfer from an amine donor to a carbonyl compound. Operation of TAs depends on pyridoxal-5′-phosphate (PLP) cofactor, which serves as electron and nitrogen shuttle. The most often employed applications of TAs are stereoselective processes targeting a single enantiomer of a chiral amine. The reactions catalyzed by a single TA and aiming chiral amine preparation can be divided into two different type of fashions: (i) in an asymmetric synthesis (AS) starting from a prochiral ketone the amine transfer from a suitable amine donor produces a chiral amine (S. Mathew et al. ACS Catal. 8(12), 993-1001 (2012), DOI: 10.1021/cs300116n; D. Koszelewski et al. Trends Biotechnol. 28(6), 324-332 (2010), DOI: 10.1016/j.tibtech.2010.03.003), while (ii) in a kinetic resolution (KR) one preferred enantiomer of a racemic amine acts as an amine donor to a simple amine acceptor, thereby resulting in a mixture of the residual enantiomer of the racemic amine and the ketone corresponding to the reacting enantiomer (D. Pressnitz et al. ACS Catal. 3(4), 555-559 (2013), DOI: 10.1021/cs400002d).

The application of these approaches depends on accessibility of starting materials, either ketones or racemic amines. From the practical point of view (achievable 100% conversion), asymmetric synthesis is rather preferred type of reaction, but due to the often unfavored thermodynamic equilibrium of the asymmetric reaction direction, the kinetic resolution is more convenient. Despite some thermodynamic problems, substrate- and product inhibition phenomena, which can stymie its dominance, the above processes for chiral amine production indicate that industrial application of TAs is becoming more widespread.

The preparation method disclosed by C. K. Savile et al. (Science 329(5989), 305-309 (2010), DOI: 10.1126/science.1188934) illustrates the applicability of TAs to perform industrial manufacturing. The process by Merck & Co. and Codexis using the CodeEvolver® protein engineering technology is developed in connection with (R)-Sitagliptin (marked as Januvia) drug used in the treatment of diabetes. To achieve the synthetic goals, a new variant of an initial TA was developed with substrate walking, modelling, and mutation approach in the first-round followed by directed evolution to a mature TA form by which the original rhodium-catalyzed asymmetric enamine hydrogenation (at 250 psi) could be replaced and the whole synthesis could be shortened. To sum up, 27 mutations were performed on the parent (R)-selective ATA117 (Arthrobacter sp.) transaminase to get the final biocatalyst with an extended binding pocket against bulky type substrates, having four orders of magnitude improved activity over the initial ATA117 and increased tolerance of iPrNH₂ (0.5-1 M), organic solvent (5-50% DMSO), heat (45° C.) and ketone concentration (100 g/L, 250 mM). Compared to the metal catalyzed process, this TA-catalyzed process (6 g/L transaminase could convert 200 g/L of prositagliptin ketone) provided (R)-sitagliptin with >99.9% enantiomeric excess, a 10 to 13% increase in overall yield, a 53% increase in productivity, and 19% reduction in total waste.

With the aid of two TAs of opposite enantiomer preference, conversion of a racemic amine is feasible to either of the enantiomers by the so called deracemization (N. J. Turner, Curr. Opin. Chem. Biol. 14(2), 115-121 (2010), DOI: 10.1016/j.cbpa.2009.11.027). Deracemization with the two stereocomplementary TAs comprises a kinetic resolution step with one TA followed by an asymmetric synthesis of the forming ketone by the stereocomplementary TA. Considering the interconversion of one amine enantiomer to the opposite enantiomer through the corresponding ketone as racemization, the deracemization process can be termed also as dynamic kinetic resolution (DKR).

In the publication of D. Koszelewski et al. (Eur. J. Org. Chem. (14), 2289-2292 (2009), DOI: 10.1002/ejoc.200801265), deracemization of α-chiral primary amines is performed by a two-step, one-pot cascade process consisting the two stereocomplementary TAs. Having performed the kinetic resolution (first) step, the TA is destructed by heat-shock and the second TA with opposite stereopreference is added with lactate dehydrogenase in a coupled reaction system to shift the reaction equilibrium to the desired amine side. Either enantiomeric form of the racemic amine can be synthesized by switching the application order of the two different stereoselective TAs.

The publication of G. Shin et al. (Chem. Commun. 77(49), 8629-8631 (2013), DOI: 10.1039/c3cc43348j) provides a solution for the drawback of the process mentioned above (heat inactivation of first TA in KR) by applying an amino acceptor which is accepted only by the first TA from the two opposite enantioselective TAs. In this way, one-pot one-step deracemization is developed, however still requiring at least two enzymes.

According to the publications of C. S. Fuchs et al. (Adv. Synth. Catal. 360(4), 768-778 (2018), DOI: 10.1002/adsc.201701449) and WO 2016/075082, preparation of Pregabalin and Brivaracetam is enabled by enantiocomplementary dynamic kinetic resolution processes leading to β-chiral primary amines from the corresponding racemic β-chiral aldehydes by TA-catalyzed enantiomer selective amination with a TA of proper enantiopreference coupled with chemical racemization due to imin-enamin tautomerism of the Schiff's base of the substrate with iPrNH₂.

Related also to interconversion of enantiomers, enzymatic racemization strategy for α-chiral primary amines is described in the publication of D. Koszelewski et al. (Chem. Eur. J. 17(1), 378-383 (2011), DOI: 10.1002/chem.201001602) catalyzed by a pair of stereocomplementary TAs in the presence of symmetric ketone/amine co-substrates, which serve as amino-group shuttles. Another approach being disclosed in this publication is a biocatalytic cross-racemization of two-different enantiocomplementary α-chiral primary amines via ketone intermediates by applying two stereocomplementary TAs [from Vibrio fluvialis (VfS-TA), ATA-117 or ATA-113] in the absence of additional external symmetric ketone as an amino-group shuttle and external amine donor.

According to the publication of F. Ruggieri et al. (ChemCatChem 10(21), 5012-5018 (2018), DOI: 10.1002/cctc.201801049) the racemization of (R)- or (S)-1-methyl-3-phenylpropylamine can be performed by employing two enantiocomplementary TAs [from Chromobacterium violaceum (CvS-TA) and from Aspergillus oryzae (AoR-TA)] and a minimum amount of pyruvate/alanine as a co-substrate.

In addition to enantiotope selectivity (in asymmetric amination of a prochiral ketone with enantiotopic sides) and enantiomer selectivity (in kinetic resolution by deamination of a racemic α-chiral primary amine) aiming preparation of enantiopure chiral amines, the somewhat similar diastereotope selectivity (in asymmetric amination of a ketone with diastereotopic sides) or diastereomer selectivity (in diastereomer resolution by deamination of a diastereomeric mixture of an α-chiral/prochiral primary amine) of TAs have also been used in several cases to perform various synthetic goals (for definition of these types of stereoselectivities, see L. Poppe et al. Stereochemistry and Stereoselective Synthesis, Wiley-VCH Verlag KGaA, Weinheim (2016), pp. 127-129).

Stereochemically more complex cases are the TA-catalyzed transformations of racemic ketones in which enantiomer selectivity (between the two enantiomers of the racemic ketone) can be manifested in parallel to diastereotope selectivity (between the Re and Si sides at the prochiral center of the C-atom of the ketone). Depending on which type of selectivity is manifested and which compounds can be equilibrated chemically or enzymatically, TA-catalyzed protocols termed as dynamic kinetic resolution are developed, for instance the synthesis of α-alkyl β-amino amides according to the publication of A. Mourelle-Insua et al. (Catal. Sci. Technol. 9(15), 4083-4090 (2019), DOI: 10.1039/C9CY01004A), in which the starting α-alkyl R-keto-amides are converted to the α-alkyl 3-amino amides with high enantiomeric excess (due to the high diastereotope selectivity) and moderate to high diastereomeric ratio (due to the moderate to good enantiomer selectivity with various degrees of chemical racemization).

In their work, J. Limanto et al. (Org. Lett. 16, 22716-22719 (2014), DOI: 10.1021/o1501002a) use TA-catalysis to transform a racemic ketone selectively to the intermediate of drug Vernakalant. Here, too, two selectivities [enantiomer selectivity (distinguishing enantiomers of the racemic ketone) and diastereotope selectivity (at carbonyl group sides)] can occur in parallel. The enantiomerically pure preparation of Vernakalant requires that, in addition to both selectivities, racemization (i.e., dynamic isomerization) of ketone enantiomers also occur under pH˜10 conditions. The corresponding TA variant, showing both high enantiomer and diastereotope selectivity, is produced by directed evolution to form the trans-isomer with high selectivity and isolated yields above 80% (trans:cis>95:1, >99.5% ee).

Another example for TA-catalyzed DKR-like process is disclosed by Z. Peng et al. (Org. Lett. 16, 860-863 (2014), DOI: 10.1021/o1403630g). Starting from a racemic 2-substituted 4-piperidone derivative the corresponding trans-amine is prepared the from using the ATA-036 transaminase (in diastereomeric ratio of 10:1 with 99% ee). The process is enantiomer selective (resolution) and diastereotope selective (amination) for the ketone. Racemization of the residual ketone by the ring-chain tautomerism of the substrate enables a DKR process resulting in 85% conversion of the racemic ketone to the desired (2R,4R)-amine.

Besides Cariprazine with trans-arrangement due to the presence of two centers of pseudoasymmetry (1r,4r), further drugs such as Sertraline, Dasotraline contain 4-substituted cyclohexaneamine units with cis- or trans-arrangement in their structure.

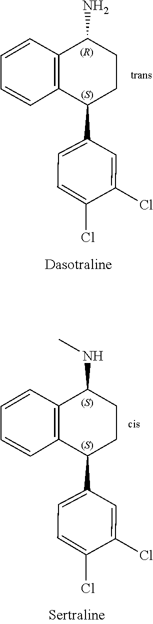

Due to the fused aromatic ring in the bicyclic system breaking the planar symmetry of the 4-substituted cyclohexaneamine/cyclohexanone systems, however, the presence of two real centers of asymmetry resulting in four possible stereoisomers are present in these drugs. To distinguish between the four possible stereoisomers, it is important to be able to selectively form the appropriate stereogenic centers on the ring or to have enantiomeric/diastereomeric separation.

The work of D. P. Gavin et al. (Sci. Rep. 9, 20285 (2019), DOI: 10.1038/s41598-019-56612-7) showed that TA-catalyzed deamination of racemic amines with an additional distant stereocenter simplifies to a KR process if both the possible enantiomer and diastereotope selectivities are high but the distant stereocenter is stable. Both a newly isolated Pseudovibrio sp. transaminase (PsS-TA) and the previously known CvS-TA have been probed to produce demethylated Sertraline diastereomers (cis- and trans-Norsertraline in Scheme 1).

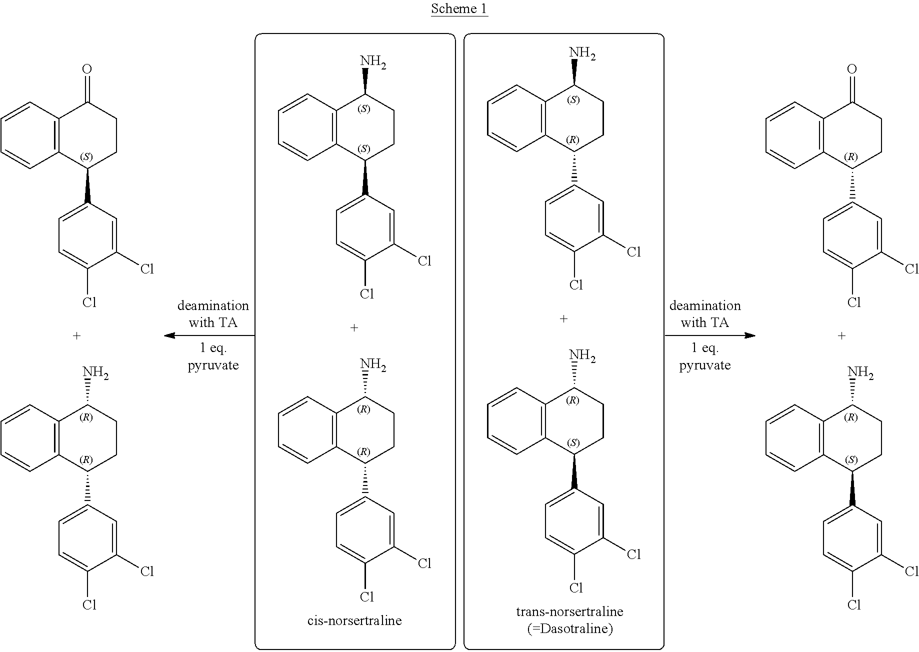

The KR by deamination from the racemic cis-isomer resulted in excellent conversion with both TAs (due to the high degree of enantiomer selectivity), leaving the (1R,4R)-isomer in high purity (ee>99%) along with the resulting (S)-ketone (due to the high degree of diastereotope selectivity). In the case of the racemic trans-isomer, only CvTA showed significant activity, where after about 50% conversion the (R)-ketone formed from the (1S,4R)-isomer (with ee˜80%), while the remaining (1R,4S)-amine was obtained with ˜95% enantiomeric excess. The processes show that both (S)-selective TAs are much more sensitive to the configuration of the amine-binding stereogenic element [(S)-selective]than to the arrangement of the more distant stereocenter.

It is disclosed in US2019/0390235 that Dasotraline can be prepared from the corresponding (S)-ketone by reductive amination using (R)-selective transaminase (in this case only diastereotope selectivity is possible) resulting in the expected (1R,4S)-Dasotraline with an enantiomeric excess of more than 99%.

Although sole manifestation of diastereotope selectivity for 4-substituted cyclohexanones or diastereomer selectivity for 4-substituted cyclohexylamines with on TAs would be close analogies to the often-applied enantiotope selectivity (in reductive amination of a prochiral ketone) or enantiomer selectivity (in oxidative deamination of an α-chiral primary amine), respectively, as the above examples illustrate, we found preparation methods with TAs for variously substituted cyclohexylamines utilizing diastereotope selectivity or diastereomer selectivity only in combination with other types of selectivities.

In developing TA-catalyzed preparation methods to obtain 2-(trans-4-aminocyclohexyl)acetic acid esters (I), with special emphasis on ethyl ester (Ib), using a single TA, initially we considered two options depicted in Scheme 2 utilizing the pure manifestation of the possible diastereotope selectivity (Scheme 2A) or diastereomer selectivity (Scheme 2B) of TAs.

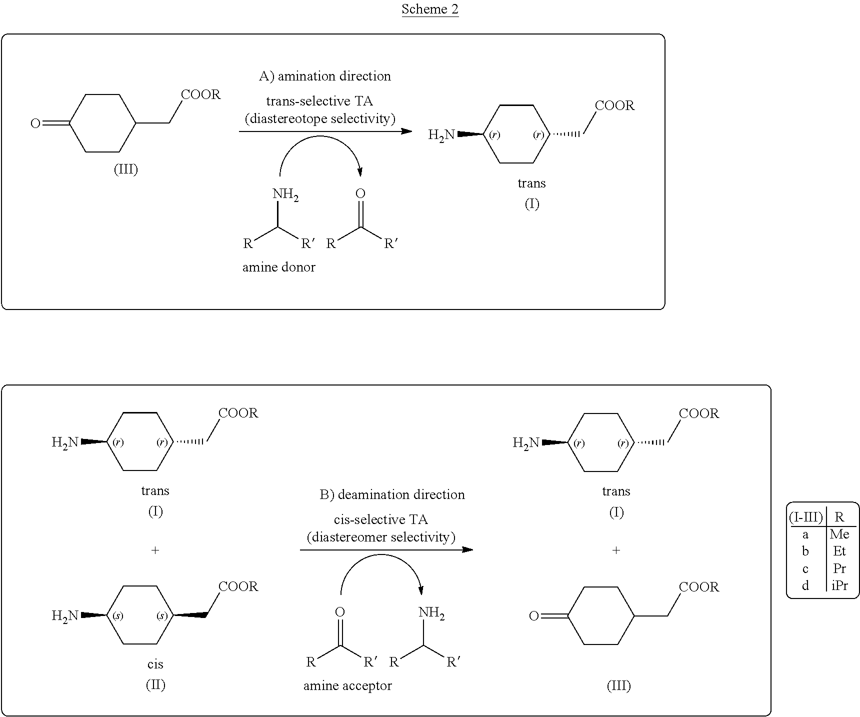

TAs of trans-selectivity (Scheme 2A) may enable conversion of an (4-alkoxycarbonyl-methyl)cyclohexanone (III) directly to the desired 2-(trans-4-aminocyclohexyl)acetic acid ester (I), while TAs of cis-selectivity (Scheme 2B) can be utilized for diastereomer resolution leading to an easy-to-separate mixture of the desired 2-(trans-4-amino-cyclohexyl)acetic acid ester (I) and the corresponding (4-alkoxycarbonylmethyl)cyclohexanone (III) forming from the cis-isomer (II).

Our screen targeting option A) with three (S)-selective TAs and three (R)-selective TAs for diastereotope selective amination of (4-ethoxycarbonylmethyl)cyclohexanone (compound IIIb) using the corresponding enantiomer of α-methylbenzylamine as amine donor revealed no TAs of trans-selectivity suitable for direct preparation of to the desired 2-(trans-4-aminocyclohexyl)acetic acid ethyl ester (compound Ib). Although no TAs with selectivity required for the formation of trans-ester (compound I) is found, the TAs with good cis-selectivity are suitable for preparation of the less easily available cis-ester (compound II) in good diastereomeric excess.

The cis-diastereomer preference is the requirement for option B) enabling preparation of trans-isomer (compound I) by diastereomer separation of the trans/cis-isomeric mixture of amines (compounds I+II). Fortunately, high degree of cis-selectivity in the amination from (4-ethoxycarbonylmethyl)cyclohexanone (compound IIIb) was found with VfS-TA and with a CvS-TA variant resulting in low to moderate conversion, respectively, with good diastereomeric excess (>90% de_{cis}). Because the diastereomeric mixture of trans/cis-amine (compounds I+II) is more readily available than the corresponding ketone (compound III), isomer separation of the diastereomeric mixture (compounds I+II) seems to be a viable process.

Because in the analogous enantiomer selective processes aiming enantiopure chiral amine preparations and starting from racemic compounds either application of stereocomplementary TAs or non-enzymic racemization due to a chemically sensitive unit in the substrate was required to surpass the conversion limitation of kinetic resolution to the reacting-enantiomer content, thus leading to a dynamic kinetic resolution or deracemization, no such effect was expected in isomer separation of the diastereomeric mixture with a single TA.

Our diastereomer separation experiments with the cis-selective TAs (a CvS-TA variant and VfS-TA) utilizing pyruvate as amine acceptor for deamination of the cis-diastereomer content of the isomeric 2-(4-aminocyclohexyl)acetic acid ethyl esters (compounds Ib+IIb) led to unexpected and surprising results. At certain reaction times, depending on the nature of the TA biocatalyst and type and amount of amine donor, a mixture of the forming ketone (compound IIIb) in lower amount than the consumed the cis-isomer (compound IIb) and diastereomerically enriched trans-isomer (compound Ib, up to >98% deₜᵣₐₙₛ) in higher amount than the original trans-isomer (compound Ib) content was obtained (molar fraction of Ib, xₜᵣₐₙₛ raised up to >85%). These results indicated an unexpected dynamic isomerization of the cis-isomer content (molar fraction of compound IIb: x_{cis}) to the trans-diastereomer (compound Ib) by a single TAs exhibiting high cis-selectivity in the diastereotope selective ketone amination direction.

Thus, the present invention relates to single transaminase catalyzed dynamic isomerization of the trans/cis-diastereomeric mixture of 4-substituted cyclohexanel-amines (C+T) for the synthesis of trans-4-substituted cyclohexane-1-amines (T) that can be performed either in batch or in continuous-flow mode.

### SUMMARY OF THE INVENTION

The process according to the present invention relates to the production of (1r,4r)-4-substituted cyclohexane-1-amines [=trans-4-substituted cyclohexane-1-amines] of formula (T) starting from a diastereomeric mixture of 4-substituted cyclohexane-1-amines (formula C+formula T)

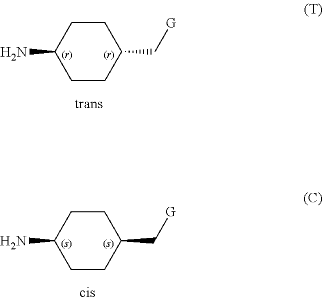

or any salt of them, where in formula (T) and in formula (C) G represents a substituent, selected from
a hydrogen atom;
a C₁₋₆ alkyl group;
an ester moiety (—COOR), where R represents a suitable alkyl, aralkyl or aryl group, preferably a C₁₋₆ alkyl group, more preferably a substituent selected from methyl, ethyl, propyl and isopropyl group;
a CH₂—OR′ group, where R′ represents hydrogen atom, or a hydroxyl protecting group;
a protected aldehyde group of formula

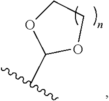

where n is an integer of 1 to 2;
a substituted or unsubstituted aryl group, preferably phenyl group; or
an aralkyl group, preferably benzyl group
   in such a way that the diastereomeric mixture is reacted with a single transaminase biocatalyst in whole-cell, soluble or immobilized form in the presence of an amine acceptor used in sub-equimolar up to equimolar quantities.

In the first aspect, the present invention relates to the production of 2-(trans-4-aminocyclohexyl)acetic acid esters of formula (I) starting from a diastereomeric mixture of 2-(4-aminocyclohexyl)acetic acid esters (formula (I)+formula (II)) or any salt of them using a single transaminase biocatalyst in whole-cell, soluble or immobilized form in the presence of an amine acceptor used in sub-equimolar up to equimolar quantities.

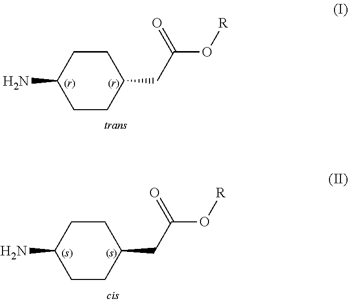

In the second aspect, the present invention relates to the production of hydroxyl-protected or protective group-free trans-4-(2-hydroxyethyl)cyclohexan-1-amines of formula (IV) starting from the corresponding trans/cis-diastereomeric mixture (compounds IV+V) or any salt of them using a single transaminase biocatalyst in the presence of an amine acceptor used in sub-equimolar up to equimolar quantities.

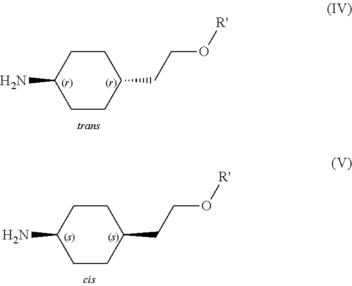

In the third aspect, the present invention relates to the production of protected 2-(trans-4-aminocyclohexyl)acetaldehydes (VII) from the corresponding trans/cis-diastereomeric mixture (compounds VII+VIII) using a single transaminase biocatalyst in the presence of an amine acceptor used in sub-equimolar up to equimolar quantities.

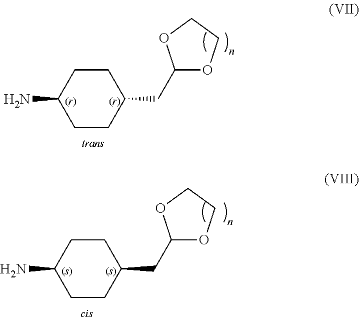

The process according to the present invention is feasible in both batch and continuous operation.

### DETAILED DESCRIPTION OF THE INVENTION

The technological solution according to the present invention is, in general terms, suitable to produce (1r,4r)-4-substituted cyclohexane-1-amines [=trans-4-substituted cyclohexane-1-amines] of formula (T) starting from a diastereomeric mixture of 4-substituted cyclohexane-1-amines (formula C+formula T) as stated above. However, the invention includes several aspects and embodiments of particular interest.

Primarily, the present invention provides a process comprising a single transaminase catalyzed dynamic isomerization of the trans/cis-diastereomeric mixture of 2-(4-aminocyclohexyl)acetic acid esters (I+II) for the synthesis of 2-(trans-4-aminocyclohexyl)acetic acid esters (I). But at the same time the dynamic isomerization processes may also applicable for preparations of trans-4-(2-hydroxyethyl)cyclohexan-1-amine of formula (IVa) or trans-4-((1,3-dioxolan-2-yl)methyl)cyclohexan-1-amine of formula (VIIa) from the corresponding cis/trans-diastereomeric mixtures of 4-(2-hydroxyethyl)cyclohexan-1-amines (formula (IVa)+formula (Va)) or 4-((1,3-dioxolan-2-yl)methyl)cyclohexan-1-amines (formula (VIIa)+formula (VIIIa)), respectively with a single transaminase biocatalyst in the presence of an amine acceptor used in sub-equimolar quantities. 2-(trans-4-Aminocyclohexyl)acetic acid esters (I) are used in the synthesis of active pharmaceutical agents. Specifically, for the synthesis of active pharmaceutical agents where diastereomerically pure trans-isomer forms of 2-(4-aminocyclohexyl)acetic acid C₁₋₆ alkyl esters are applied. Especially, 2-(trans-4-aminocyclohexyl)acetic acid ethyl ester products (Ib) is used in the synthesis of trans-N-{4-[2-[4-(2,3-dichlorophenyl)-piperazin-1-yl]-ethyl]cyclohexyl}-N′,N′-dimethylurea, commonly known as Cariprazine. The trans-4-(2-hydroxyethyl)cyclohexan-1-amine (IVa) or trans-4-((1,3-dioxolan-2-yl)methyl)cyclohexan-1-amine (VIIa) may also be applied in alternative synthetic processes leading to Carprazine.

In the preferably used esters, the R group in formula I represents C₁₋₆ alkyl moiety containing 1 to 6 carbon atoms with straight or branched chain.

The chemical structure of the drug substance Cariprazine contains the 4-substituted cyclohexaneamine unit. Thus, it is important to selectively form the two pseudoasymmetric centers with the appropriate spatial arrangement of the substituents on the ring [i.e. (1r,4r)]. In practice, this specifically means that only the diastereomerically pure trans-isomeric form of ethyl 2-(4-aminocyclohexyl)acetate HCl (Ib·HCl) is applicable and either selective formation of this diastereomer (Ib) or separation of the two diastereomers of the intermediate compound (Ib+IIb) is essential.

As it was mentioned above the 2-(trans-4-aminocyclohexyl)acetic acid ethyl ester hydrochloride (Ib·HCl) starting material in industrial scale is provided via simple reaction steps and in high quality by the production process according to WO2010/070368. By using this procedure, 2-(4-aminocyclohexyl)acetic acid ester derivatives, for example methyl, ethyl, or propyl ester derivatives, can be prepared as a mixture of cis/trans-isomers in a ratio of about 1:1 (compounds Ia+IIa, Ib+IIb, or Ic+IIc, respectively). This mixture (I+II) is separated to the desired trans-product (I) and cis-by-product (II) by crystallization. Usually, the cis-by-product is treated as waste or may be recycled to the separation step by isomerization to a mixture of cis- and trans-compounds (I+II).

We sought to make the practical application of a transaminase-based biocatalytic process a competitive technological solution of the diastereomer separation using either batch or continuous flow process methods.

It has surprisingly been found that this newly designed method surpassed the relatively good product yield of the traditional separation-based process limited to the original trans-isomer (compound I) contents and thereby it makes possible to produce the trans-isomer (compound I) in a much more efficient way.

We have developed a biocatalytic process by which most of the cis-isomer (compound II) content of a diastereomeric cis/trans-mixture (compounds I+1I) can be isomerized with a single TA and sub-equimolar amounts of a proper amine acceptor leading to a mixture of trans-isomer (compound I) in good yield and high diastereomeric excess and a lower amount of the corresponding 4-substituted cyclohexanone (compound III). We have succeeded in developing a method that is much more efficient than the separation processes that have been part of the traditional manufacturing process so far.

We have developed for the first time a biocatalytic route for production of 2-(trans-4-aminocyclohexyl)acetic acid esters (formula I), particularly C1-6 alkyl esters, preferably 2-(trans-4-aminocyclohexyl)acetic acid ethyl ester (compound Ib) and 2-(trans-4-aminocyclohexyl)acetic acid ethyl ester isopropyl ester (compound Id), most preferably 2-(trans-4-aminocyclohexyl)acetic ethyl ester (compound Ib).

This solution according to the present invention means new industrially applicable alternative approach for the preparation of 2-(trans-4-aminocyclohexyl)acetic acid ethyl ester HCl (compound Ib·HCl) and thus make a significant contribution to improving the production of Cariprazine since 2-(trans-4-aminocyclohexyl)acetic acid ethyl ester HCl (compound Ib·HCl) is the key intermediate of Cariprazine.

When the TA-catalyzed reductive amination of the corresponding 2-(4-oxocyclohexyl)acetic acid ethyl ester (IIIb) was probed as a diastereotope selective method with the aim of obtaining a trans-product (compound Ib) directly, the probed TAs yielded a product containing the cis-form (compound IIb) predominantly.

Our initial working hypothesis for diastereomer selective separation of the cis/trans-isomeric mixture of some C1-6 alkyl esters, preferably ethyl and isopropyl esters of 2-(4-aminocyclohexyl)acetic acid (compounds Ib+IIb, and Id+IId, respectively), was that with the aid of a cis-selective TA the resulting mixture of cis/trans-isomers in a ratio of about 1:1 (compounds I+II) could be separated by a biocatalytic method in the presence of sub equimolar amount of a ketone-type amine acceptor.

We have also surprisingly found that the aimed separation of the isomeric esters (compounds I+II) through applications of biocatalytic processes using TAs in the presence of pyruvate as amine acceptor compound in sub-equimolar amounts could be accomplished with excellent results yielding up to >85% trans-isomer (compound I) with >98% diastereomeric excess and <15% ketone (compound III) and that the processes which could be carried out either in batch mode or in a continuous flow mode involved various degrees of cis- to trans-isomerization (conversion of compound II to compound I) besides deamination of the cis-isomer (compound II) to ketone (compound III).

For the single TA-catalyzed biocatalytic cis- to trans-isomerization process starting from the cis/trans-2-(4-aminocyclohexyl)acetic acid ester (compounds I+II), preferably ethyl ester isomeric mixture (compounds Ib+IIb), as amine acceptor compound in sub-equimolar amounts 2-(4-oxycyclohexyl)acetic acid esters (III), preferably 2-(4-oxycyclohexyl)acetic acid ethyl ester (compound IIIb) could be applied leading to a mixture of trans-isomer (compound Ib) with enhanced diastereomeric excess (deₜᵣₐₙₛ˜75-80%) and the added 2-(4-oxycyclohexyl)acetic acid ester (compound IIIb) close to the initial amount. After separation the trans-isomer (compound Ib) from this mixture by recrystallization, the mixture from the mother liquor of the recrystallization containing cis-isomer (compound IIb) and the amine acceptor ketone (compound IIIb) can be directly recycled into the next isomerization step as amine acceptor/starting diastereomer mixture.

Generally, non-stereoselective reductive amination of ketones with various functional groups, for example aromatic, aliphatic and carboxylate groups can be accomplished either in batch or in continuous flow mode according to the method described in the publication of P. Falus et al. (Tetrahedron Lett., 52, 1310-1312 (2011), DOI: 10.1016/j.tetlet.2011.01.062).

During our development work, a 2-(4-oxycyclohexyl)acetic acid ester (I), preferably 2-(4-oxycyclohexyl)acetic acid ethyl ester (IIIb) or 2-(4-oxycyclohexyl)acetic acid isopropyl ester (IIId), most preferably 2-(4-oxycyclohexyl)acetic acid ethyl ester (compound IIIb) was tried as amine acceptor for the biocatalytic cis- to trans-isomerization (compound II to compound I) process.

Six transaminase enzymes with different enantiomeric preferences having already been successfully applied for kinetic resolution of racemic amines in immobilized whole-cell form as disclosed by Z. Molnar et al. (Catalysts, 9, 438 (2019), DOI: 10.3390/cata19050438) were considered for the stereoselective processes. The selected TAs included three (R)- and three (S)-selective TAs, the (R)-selective TAs from Arthrobacter sp. (ArR-TA), its mutated variant (ArRmut-TA), Aspergillus terreus (AtR-TA); and the (S)-selective TAs from Arthrobacter citreus (ArS-TA), a mutated variant of Chromobacterium violaceum (CvS_{W60C}-TA), Vibrio fluvialis (VfS-TA), respectively.

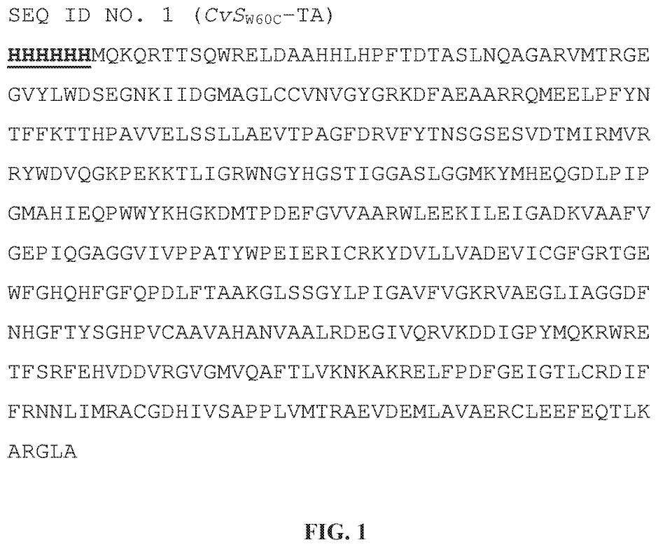

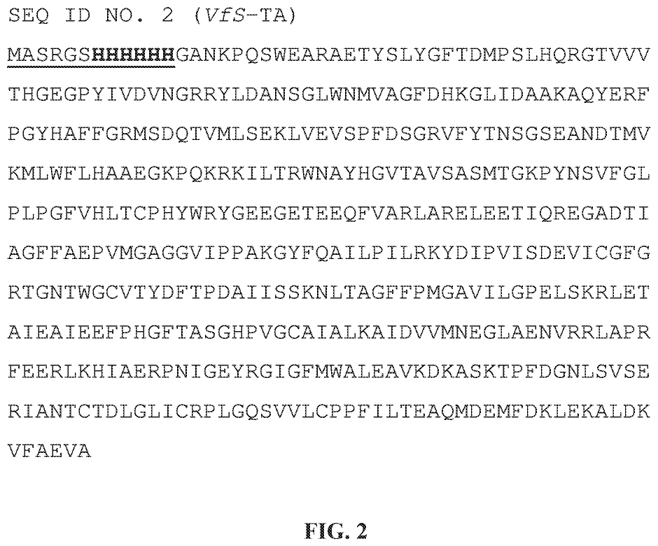

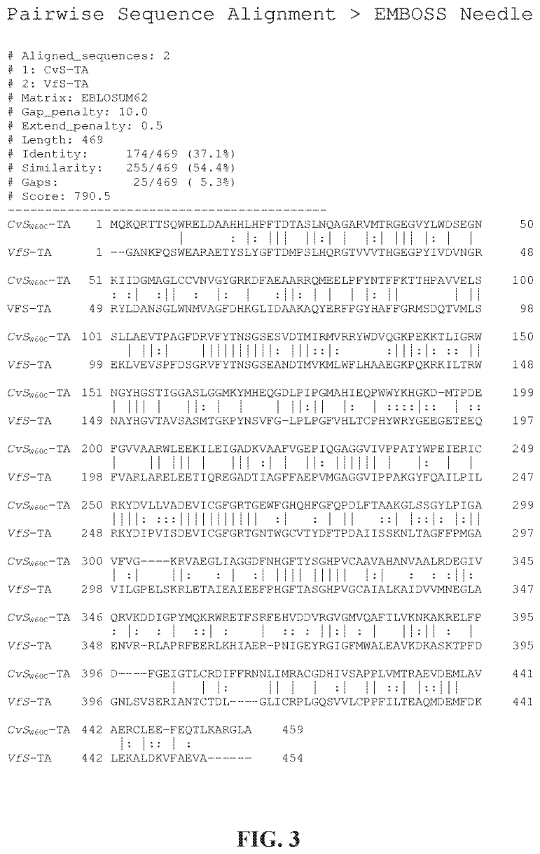

Cyclohexanones substituted with alkoxycarbonylmethyl groups at the 4-position (formula III) were transformed in batch with the six (R)- or (S)-selective transaminases to the diastereomers of the corresponding alkyl 2-(4-aminocyclohexyl)acetate (formula I or formula II). The trials starting from ethyl 2-(4-oxycyclohexyl)acetate (compound IIIb) revealed the highest diastereotope selectivity preferring the formation of cis-diastereomer (compound IIb, de_{cis}>90%) by the TA from Chromobacterium violaceum (CvS_{W60C}-TA) [K. E. Cassimje et al. (Org. Biomol. Chem., 10, 5466-5470 (2012), DOI: 10.1039/C20B25893E)] and Vibrio fluvialis (VfS-TA) [F. G. Mutti et al. (Eur. J. Org. Chem., 1003-1007 (2012), DOI: 10.1002/ejoc.201101476]. Thus, the cis-selective TAs include but are not limited to the Chromobacterium violaceum TA mutant W60C (CvS_{W60C}-TA), and to the Vibrio fluvialis TA (VfS-TA) characterized by their amino acid sequences. The amino acid sequence of CvS_{W60C}-TA (SEQ ID NO. 1) is shown by FIG. 1; the amino acid sequence of VfS-TA (SEQ ID NO. 2) is shown by FIG. 2. A pairwise sequence alignment for CvS_{W60C}-TA and VfS-TA is shown by FIG. 3. The underlined amino acids in the exemplary sequences shown by FIG. 1 and FIG. 2 encode affinity tags, therefore they are not involved in sequence comparisons.

Because the two amino acid sequences of the exemplary TAs with cis-selectivity (SEQ ID NO. 1 for CvS_{W60C}-TA and SEQ ID NO. 2 for VfS-TA as depicted in FIGS. 1 and 2, respectively) share 37.1% sequence identity (see FIG. 3), any TAs with higher than 40% sequence identity to either SEQ ID NO. 1 or SEQ ID NO. 2 is expected to have similar catalytic properties.

The invention provides a dynamic isomerization process for converting a cis-4-substituted cyclohexane-1-amine (characterized by formula C) to the corresponding trans-4-substituted cyclohexane-1-amine (characterized by formula T) by a single transaminase comprising an amino acid sequence with at least about 37%, 40%, 50%, 55%, 60%, 70%, 75%, 80%, 85%, 90%, 91%, 92%, 93%, 94%, 95%, 96%, 97%, 98%, 99%, or more sequence identity to any of the exemplary sequences of the invention (SEQ ID NO. 1 for CvS_{W60C}-TA or SEQ ID NO. 2 for V/S-TA) over a region of at least about 100 residues, wherein the sequence identities are determined by analysis with a sequence comparison algorithm or by visual inspection, to an amino acid sequence of the invention.

According to our experimental results, the TA-catalyzed reaction aiming diastereomer selective kinetic separation from the cis/trans-isomeric mixture of some C1-6 alkyl esters (I+II), preferably ethyl and isopropyl esters of 2-(4-aminocyclohexyl)acetic acid (Ib+IIb and Id+IId, respectively) with the aid of a single cis-selective TA in the presence of sub-equimolar amount of a ketone-type amine acceptor can be accomplished yielding trans-isomeric product (compound I) of high diastereomeric purity (deₜᵣₐₙₛ) and in yields significantly higher than the trans-isomer (compound I) content (xₜᵣₐₙₛ) of the original cis/trans-isomeric mixture (compounds I+II). Therefore, our invented process is not a simple diastereomer selective kinetic separation of the cis/trans-isomeric mixture (compounds I+II) but a dynamic isomerization process converting a significant proportion of the cis-isomer by-product (compound II) to the desired trans-isomeric product (compound I).

Without adhering to any scientific background theory in relation to the present invention, the explanation for the ongoing chemical process might be that in presence of an amine acceptor in sub-equimolar amounts, the cis-isomer (compound II) H ketone (compound III) transformation is more favorable kinetically (relatively fast in both directions and therefore reversible), while the ketone (compound III) H trans-isomer (compound I) transformation is much slower, and its equilibrium is shifted in the direction of thermodynamically favored trans-isomer (compound I).

In view of the first aspect of the invention we sought to make the practical application of a transaminase-based biocatalytic process a technological solution of the diastereomer separation using batch method.

Using sodium pyruvate as amine acceptor in sub-equimolar amounts for the dynamic isomerization process starting from trans/cis-amine (compounds Ib+IIb in nearly 1:1 ratio, 25 mM), high diastereomeric excess of the trans-isomeric product (compound Ib: deₜᵣₐₙₛ>95%) can be reached with both Chromobacterium violaceum and Vibrio fluvialis TAs (CvS_{W60C}-TA and VfS-TA, respectively) at different reaction times, depending on the TA biocatalyst formulation and amount (Table 1).

TABLE 1

| | TA biocatalyst | Time | deₜᵣₐₙₛ | xₜᵣₐₙₛ | x_{cis} | xₖₑₜₒₙₑ |
|---|---|---|---|---|---|---|
| Example | (amount) | (h) | (%) | (%) | (%) | (%) |
| Example 1 | CvS-TA imm. w. cell | 0 | 12.0 | 56.0 | 44.0 | 0.0 |
| | (25 mg/mL) | 24 | 98.3 | 76.3 | 0.6 | 23.0 |
| Example 2 | VfS-TA imm. w. cell | 0 | 12.0 | 56.0 | 44.0 | 0.0 |
| | (25 mg/mL) | 24 | 96.0 | 70.6 | 1.5 | 27.9 |
| Example 3 | VfS-TA imm. w. cell | 0 | 12.0 | 56.0 | 44.0 | 0.0 |
| | (50 mg/mL) | 6 | 97.3 | 74.5 | 1.0 | 24.5 |
| Example 4 | CvS-TA purified | 0 | 12.0 | 56.0 | 44.0 | 0.0 |
| | (0.5 mg/mL) | 2 | 99.9 | 86.0 | 0.0 | 14.0 |
| Example 5 | VfS-TA purified | 0 | ~0 | 49.0 | 51.0 | 0.0 |
| | (0.5 mg/mL) | 3 | 98.7 | 79.0 | 0.5 | 20.5 |
| Example 6 | VfS-TA covalently imm. | 0 | ~0 | 49.0 | 51.0 | 0.0 |
| | (5 mg/mL) | 6 | 95.9 | 74.9 | 1.6 | 23.6 |

Example 4 in Table 1 shows the best setup with purified soluble CvS_{W60C}-TA starting from a mixture of trans-isomer (compound Ib, xₜᵣₐₙₛ=56%) and cis-isomer (compound IIb, x_{cis}=44%) yielding after 2 h reaction time a good yield to trans-isomer (compound Ib: xₜᵣₐₙₛ=86%) with deₜᵣₐₙₛ>99% and a smaller amount of ketone (compound IIIb: xₖₑₜₒₙₑ=14%) in batch mode. This result demonstrates that up to 68% of the original cis-isomer (compound IIb) content could be converted to the desired trans-isomer (compound Ib).

The Examples presented in Table and FIG. 1 showed that significant degree of cis- to trans-isomerization [conversion of 34-68% of the original cis-content (compound IIb) to the desired trans-isomer (compound Ib)] could be achieved with either the various forms of CvS_{W60C}-TA (Examples 1 and 4) or with different preparations of VfS-TA (Examples 2, 3, 5 and 6).

The Examples listed in Table 1 also shows that the cis-selective TAs can be applied as biocatalysts in their purified soluble forms (Examples 4 and 5) or in their immobilized forms such as TA-expressing whole-cells immobilized by sol-gel entrapment (according to the method of Z. Molnir et al., Catalysts, 9, 438 (2019), DOI: 10.3390/cata19050438) (Examples 1, 2 and 3) or as purified protein attached covalently to porous polymeric resin (according to the method of E. Abahazi, et al., Biochem. Eng. J. 132, 270-278 (2018), DOI: 10.1016/i.bej.2018.01.022) (Example 6).

When starting from trans/cis-amine (compounds Ib+IIb in nearly 1:1 ratio, 25 mM) and using 4-substituted cyclohexanone (compound IIIb) or cyclohexanone as amine acceptor, a thermodynamic equilibrium (deₜᵣₐₙₛ˜75-80%) could be reached after different reaction times, depending on the form and amount of the TA biocatalyst (Table 2).

TABLE 2

| | TA biocatalyst, amount | Time | deₜᵣₐₙₛ | xₜᵣₐₙₛ | x_{cis} | xₖₑₜₒₙₑ |
|---|---|---|---|---|---|---|
| Example | (amine donor, amount) | (h) | (%) | (%) | (%) | (%) |
| Example 7 | VfS-TA imm. w cell, 50 mg/mL | 0 | 13.6 | 51.5 | 39.2 | 11.0 |
| | (0.1 eq. IIIb) | 48 | 71.5 | 74.6 | 12.4 | 13.0 |
| Example 8 | VfS-TA purified, 0.5 mg/mL | 0 | ~0 | 44.4 | 46.2 | 11.0 |
| | (0.1 eq. IIIb) | 48 | 75.7 | 78.4 | 10.8 | 10.7 |
| Example 9 | VfS-TA purified, 0.5 mg/mL | 0 | ~0 | 49.0 | 51.0 | 0.0 |
| | (0.2 eq. cyclohexanone) | 48 | 80.4 | 85.9 | 9.3 | 4.8 |

Example 7 in Table 2 shows that with immobilized whole cell form of VfS-TA and using the corresponding 4-substituted cyclohexanone (compound IIIb: xₖₑₜₒₙₑ=11%) as amine acceptor, the dynamic isomerization process starting from a trans/cis-diastereomeric mixture (compound Ib and compound IIb, in 51.5:39.2 ratio) can afford besides nearly the original amount of ketone (compound IIIb: xₖₑₜₒₙₑ=13.0%) a significantly increased amount of the trans-isomer (compound Ib: xₜᵣₐₙₛ=74.6% with deₜᵣₐₙₛ=71.5%) after 48 h reaction time in batch mode. This data means that with the immobilized VfS-TA biocatalyst ˜53% of the original cis-isomer (compound IIb) content could be converted to the desired trans-isomer (compound Ib). A trans/cis-separation by the method analogous to that disclosed in WO2010/070368, can result in a mixture from the mother liquor containing almost identical amounts of cis-isomer (compound IIb) and ketone (compound IIIb) which can be recycled as amine acceptor/cis-isomer mixture in a next dynamic isomerization cycle.

Example 8 in Table 2 shows that with purified soluble VfS-TA and using the corresponding 4-substituted cyclohexanone (compound IIIb: xₖₑₜₒₙₑ=11%) as amine acceptor, the dynamic isomerization process starting from a mixture of trans-isomer (compound Ib, xₜᵣₐₙₛ=44.4%) and cis-isomer (compound IIb, x_{cis}=46.6%) can result after 48 h reaction time in a significant increase of the amount of the trans-isomer (compound Ib: xₜᵣₐₙₛ=78.4%) with a minor amount of residual cis-isomer (compound IIb: x_{cis}=10.8%) meaning deₜᵣₐₙₛ=75.7% along with the original amount of ketone (compound III: xₖₑₜₒₙₑ=10.7%) in batch mode. This data means that ˜53% of the original cis-isomer (compound IIb) content could be converted to the desired trans-isomer (compound Ib).

Example 9 in Table 2 indicates that with purified soluble VfS-TA and cyclohexanone (in 0.2 eq. amount) as amine acceptor, the dynamic isomerization process starting from a mixture of trans-isomer (compound Ib, xₜᵣₐₙₛ=49%) and cis-isomer (compound IIb, x_{cis}=51%) can result after 48 h reaction time the highest increase of the amount of the trans-isomer (compound Ib: xₜᵣₐₙₛ=85.9%) with a minor amount of residual cis-isomer (compound IIb: x_{cis}=9.3%) meaning deₜᵣₐₙₛ=80.4% in batch mode. This deₜᵣₐₙₛ value is presumably close to the highest achievable thermodynamic equilibrium ratio.

In view of the second aspect of the invention we sought to make the practical application of a transaminase-based biocatalytic process a technological solution of the diastereomer separation using continuous-flow mode.

The use of flow chemistry is witnessing an ever-increasing rise in synthetic methodologies (M. Giudi et al. Chem. Soc. Rev. 49, 8910-8932 (2020), DOI: 10.1039/c9cs00832b). The broad versatility in applications is thanks to the modular nature of the approach, allowing for the facile integration of new conditions, equipment, analytics, automation, and types of reagents for both single and multistep processes. Precise control results in excellent reproducibility and safety—making flow chemistry applicable to a range of disciplines also in syntheses for pharmaceutical industry (M. Baumann et al. Org. Proc. Res. Dev. 24, 1802-1813 (2020), DOI: 10.1021/acs.oprd.9b00524; D. L. Hughes, Org. Proc. Res. Dev. 24, 1850-1860 (2020), DOI: 10.1021/acs.oprd.0c00156).

The benefits of synthetic chemistry in continuous flow systems can be combined with the benefits of biocatalysis, including reactions under greener, milder, lower temperature, and aqueous conditions (J. Britton et al., Chem. Soc. Rev. 47, 5891-5918 (2018), DOI: 10.1039/c7cs00906b; P. De Santis et al., React. Chem. Eng. 5, 2155-2184 (2020), DOI: 10.1039/d0re00335b). In addition to fine control of the reaction conditions, use of a continuous flow system can alleviate the challenges of enzyme catalysis, such as substrate and product inhibition. Reactions using cells and enzymes can take advantage of significantly improved mixing, mass transfer, thermoregulation, feasibility of pressure reactions, automation, and reduced process variability in continuous systems, as well as product analysis and purification facilitated by continuous flow. Thus, the combination of continuous flow and biocatalysis has emerged as a highly efficient approach to achieve various synthetic goals.

The selection criteria of the three ideal reactor types, namely the batch stirred tank reactor (BSTR) for biocatalysis is analyzed by J. M. Woodley (Woodley, J. M. Chapter 3.9. in “Science of Synthesis: Biocatalysis in Organic Synthesis”, K. Faber, W. D. Fessner, N. J. Turner, Eds.; Thieme: Stuttgart (2015), Volume 3, pp. 515-546, DOI: DOI: 10.1055/sos-SD-216-00331) and R. Lindeque et al. (Catalysts 9, 262 (2019); DOI: 10.3390/cata19030262).

BSTRs are commonly used for biocatalytic reactions due to their simplicity and flexibility. In a BSTR, first the substrate and enzyme are filled into a mechanically stirred tank, to initiate the reaction, after which no material is removed until the reaction is stopped. In BSTRs the concentrations are the same regardless of location within the reactor. At first, the substrate is initially consumed quickly, whilst later in the reaction the reaction rate slows. However, given sufficient time in the BSTR, complete conversion can be achieved, provided the equilibrium is favorable.

The design of a continuously stirred tank reactor (CSTR) is similar to that of a BSTR, except that material is continuously added to, and removed from, thus the working volume remains constant. In CSTR, the biocatalyst is either fed continuously to the reactor to balance the loss of catalyst in the effluent or it is retained within the reactor by immobilization and/or partially permeable membranes. Because CSTRs are well-mixed, the reactor contents and effluent are homogenous. However, in a CSTR the effluent will always contain some substrate and so full substrate conversion is not possible. This trade-off between reaction rate and conversion is an important characteristic of CSTRs.

In a continuous plug flow reactor (CPFR), reactants are pumped into a long tubular reactor where, unlike stirred tanks, material flowing through does not mix with any material flowing ahead of it, or behind it. This results in concentration gradients over the length of the reactor, identical to the concentration gradients over time in a BSTR. Therefore, if the reactor is sufficiently long, the substrate can be fully converted. For this reason, the time material spends in a CPFR is simply a function of the reactor length and volumetric flowrate. Although it is possible to operate a CPFR with a soluble catalyst, biocatalysts are typically immobilized onto the reactor wall or on particles of a carrier material, which are then packed into a tube to form a continuous packed-bed reactor (CPBR).

When we intended to produce the trans-isomer (formula I) in the continuous-flow mode, we performed experiments aiming the dynamic isomerization yielding 2-(trans-4-amino-cyclohexyl)acetic acid ester (formula I) from the trans/cis-isomeric mixture (formula I+formula II) catalyzed by a TA in CPFR. The applicability of TA preparations in packed-bed reactors for continuous-flow process depends on the physicochemical properties of the immobilized TA such as enzyme density and availability on the surface, shape, and particle size of the carrier. Various immobilization methods of TAs could be used for TA-catalyzed continuous-flow kinetic resolution processes. The CvS_{W60C}-TA and VfS-TA could be applied as immobilized whole cell biocatalysts according to the method of Z. Molnir et al. (Catalysts, 9, 438 (2019), DOI: 10.3390/cata19050438), or the CvS_{W60C}-TA immobilized covalently on macroporous polymer resin was also applicable as described by E. Abahizi, et al. (Biochem. Eng. J. 132, 270-278 (2018), DOI: 10.1016/j.bej.2018.01.022).

According to a preferred embodiment of the present invention diastereomeric mixtures of ethyl or isopropyl esters of 2-(4-aminocyclohexyl)acetic acid (Ib+IIb or Id+IId, respectively) were isomerized in the presence of sub-equimolar pyruvate as amine acceptor via the formation of the ketone (compound III) intermediate to a mixture of highly diastereopure trans-amine (I, with deₜᵣₐₙₛ>99%) and the ketone (compound III), in the presence of appropriate cis-selective transaminases (e.g., Chromobacterium violaceum TA or Vibrio fluvialis TA).

According to our experiment results for dynamic isomerization, particularly an immobilized form of a cis-selective TA was advantageous, more particularly CvS_{W60C}-TA and VfS-TA were advantageous and most particularly use of CvS_{W60C}-TA immobilized covalently on macroporous polymer resin (CvS_{W60C}-TA_{CB}) was advantageous. The dynamic isomerization process starting from a solution of trans/cis-amine (compounds Ib+IIb or d+IId in nearly 1:1 ratio, 20 mM) and sodium pyruvate as amine acceptor (0.95 eq.) at 10 μL min⁻¹ flow rate in serially coupled packed bad columns filled with CvS_{W60C}-TA_{CB} (˜220 mg) resulted in excellent diastereomeric excess of the trans-product (compound Ib or Id: deₜᵣₐₙₛ>99%) (Table 3).

TABLE 3

| | CvS_{W60C}-TA_{CB} columns | Time | deₜᵣₐₙₛ | xₜᵣₐₙₛ | x_{cis} | xₖₑₜₒₙₑ |
|---|---|---|---|---|---|---|
| Example | (number) | (h) | (%) | (%) | (%) | (%) |
| Example 10 | 3 | 0 | | 30.3 | 69.7 | 0.0 |
| | | 48 | >99 | 38.8 | 0 | 61.2 |
| Example 11 | 4 | 0 | | 48.3 | 51.7 | 0.0 |
| | | 48 | >99 | 54.0 | 0 | 46.0 |

Example 10 in Table 3 shows that with CvS_{W60C}-TA immobilized covalently on macroporous polymer resin and using sodium pyruvate (0.95 eq.) as amine acceptor, the dynamic isomerization process starting from a trans/cis-diastereomeric mixture of the ethyl esters in HCl salt form (compounds Ib·HCl+IIb·HCl, in 30.3:69.7 ratio) can be performed in continuous flow mode. The non-optimized process afforded besides the intermediate ketone (compound IIIb: xₖₑₜₒₙₑ=61.2%) an increased amount of the trans-isomer (compound Ib: xₜᵣₐₙₛ=38.8% with deₜᵣₐₙₛ>99) after 48 h continuous operation. This data means that with the immobilized CvS_{W60C}-TA biocatalyst, the original 30.3% trans-isomer (compound Ib) content is increased by 8.5% to 38.8%, enabling 30.7% isolated yield for the desired trans-isomer as HCl salt (compound Ib·HCl).

Example 11 in Table 3 demonstrates that covalently immobilized CvS_{W60C}-TA biocatalyst in presence of sodium pyruvate (0.95 eq.) as amine acceptor can be applicable for the dynamic isomerization of a trans/cis-diastereomeric mixture of the isopropyl esters in their HCl salt form (compounds Id·HCl+IId·HCl, in 48.3:51.7 ratio) in continuous flow mode. The non-optimized process produced in addition the intermediate ketone (compound IIId: xₖₑₜₒₙₑ=46.0%) a risen amount of the trans-isomer (compound Id: xₜᵣₐₙₛ=54.0% with deₜᵣₐₙₛ>99) after 48 h continuous operation. These results indicate that with the immobilized CvS_{W60C}-TA biocatalyst, the initial 48.3% trans-isomer (compound Id) amount is increased by 5.7% to 54.0%, permitting 46.5% isolated yield for the required trans-isomer as HC salt (compound Id·HCl).

According to our experimental results summarized in Table 4, the partial dynamic isomerization with an immobilized form of a cis-selective TA could be carried out on a further selection of cis/trans-diastereomeric mixtures of 4-substituted cyclohexyl amines (compound C+compound T) in continuous flow mode. The dynamic isomerization process starting from a solution of trans/cis-amine (compounds C+T) and sodium pyruvate as amine acceptor (0.95 eq.) at 10 μL min^{t} flow rate and in serially coupled packed bad columns filled with CvS_{W60C}-TA_{CB} (˜220 mg) at 40° C. resulted in excellent diastereomeric excess of the trans-product (compound Ib or Id: deₜᵣₐₙₛ>99%) (Table 4).

TABLE 4

| | Substrate, amount | Time | deₜᵣₐₙₛ | xₜᵣₐₙₛ (Yield) | | x_{cis} | xₖₑₜₒₙₑ |
|---|---|---|---|---|---|---|---|
| Example | (No of CvS_{W60C}-TA_{CB} columns) | (h) | (%) | (%) | | (%) | (%) |
| Example 12 | C + T (G = H), 20 mM | 0 | | 58.0 | | 42.0 | 0.0 |
| | 4 | 24 | >99 | 80.2 | (—) | 0 | 19.8 |
| Example 13 | C + T (G = Me), 20 mM | 0 | | 34.6 | | 65.4 | 0.0 |
| | 2 | 24 | >99 | 46.2 | (30.4) | 0 | 53.8 |
| Example 14 | C + T (G = Ph), 15 mM | 0 | | 73.8 | | 26.2 | 0.0 |
| | 2 | 24 | >99 | 83.2 | (77.7) | 0 | 16.8 |
| Example 15 | C + T (G = CH₂Ph), 15 mM | 0 | | 49.3 | | 50.7 | 0.0 |
| | 1 | 24 | >99 | 59.8 | (54.1) | 0 | 40.2 |

Example 12 in Table 4 shows that starting from a cis/trans-diastereomeric mixture of 4-methylcyclohexan-1-aminium chloride (compounds C·HCl+T·HCl (G=H), in 42:58 ratio) the dynamic isomerization process can be performed with the immobilized CvS_{W60C}-TA biocatalyst in presence of sodium pyruvate (0.95 eq.) as amine acceptor in continuous flow mode. The non-optimized process afforded besides the intermediate ketone (compound K (G=H): xₖₑₜₒₙₑ=19.8%) an increased amount of the trans-isomer (compound T (G=H): xₜᵣₐₙₛ=80.2% with deₜᵣₐₙₛ>99) after 24 h continuous operation. This data shows that the original 58.0% trans-isomer (compound T (G=H)) content is increased by 22.2% to 80.2%. Due to its volatility, the product (compound T (G=H)) was not isolated.

Example 13 in Table 4 demonstrates that covalently immobilized CvS_{W60C}-TA biocatalyst in presence of sodium pyruvate (0.95 eq.) as amine acceptor can be applicable for the dynamic isomerization of a cis/trans-diastereomeric mixture of 4-ethylcyclohexan-1-aminium chloride (compounds C·HCl+T·HCl (G=Me), in 65.4:34.6 ratio) in continuous flow mode. The non-optimized process produced in addition the intermediate ketone (compound C+T (G=Me): xₖₑₜₒₙₑ=53.8%) an increased amount of the trans-isomer (compound T (G=Me): xₜᵣₐₙₛ=46.2% with deₜᵣₐₙₛ>99) after 24 h continuous operation. These results indicate that the initial 34.6% trans-isomer (compound T (G=Me)) amount is increased by 11.6% to 46.2%, permitting 30.4% isolated yield for the required trans-isomer as HC salt (compound T·HCl (G=Me)).

Example 14 in Table 4 indicates that with on resin immobilized CvS_{W60C}-TA biocatalyst and sodium pyruvate (in 0.95 eq. amount) as amine acceptor, the non-optimized dynamic isomerization process starting from a cis/trans-diastereomeric mixture of 4-phenylcyclohexan-1-aminium chloride (compounds C·HCl+T·HCl (G=Ph), in 26.2:73.8 ratio) in continuous flow mode can result after 24 h reaction time the highest amount of the trans-isomer (compound T (G=Ph): xₜᵣₐₙₛ=83.2%) enabling 77.7% isolated yield for the required trans-isomer as HCl salt (compound T·HCl (G=Ph)).

Example 15 in Table 4 validates that the dynamic isomerization process with the immobilized CvS_{W60C}-TA biocatalyst in presence of sodium pyruvate (0.95 eq.) as amine acceptor starting from a cis/trans-diastereomeric mixture of 4-benzylcyclohexan-1-aminium chloride (compounds C·HCl+T·HCl (G=CH₂Ph), in 50.7:49.3 ratio) can be achieved in continuous flow mode. The non-optimized process led to besides the intermediate ketone (compound K (G=CH₂Ph): xₖₑₜₒₙₑ=40.2%) an increased amount of the trans-isomer (compound T (G=CH₂Ph): xₜᵣₐₙₛ=59.8% with deₜᵣₐₙₛ>99) after 24 h continuous operation. This data shows that the original 58.0% trans-isomer (compound T (G=H)) content is increased by 10.5% to 59.8% allowing 54.1% isolated yield for the required trans-isomer as HC salt (compound T·HCl (G=CH₂Ph)).

In summary, all our experiments in Table 1-4 show that the dynamic isomerization of a cis/trans-diastereomeric mixture of a 4-substituted cyclohexan-1-amine (compounds C+T) or its salt (compounds C·HCl+T·HCl) to trans-diastereomer (compounds T) can be accomplished with various forms of a cis-selective TA in presence of a sub equimolar amount of a ketone serving as amine acceptor in batch or in continuous flow mode. In all cases, the amount of the trans-isomer (T) in the product mixture is significantly higher than in the original cis/trans-diastereomeric mixture (C+T) indicating the potential of the dynamic isomerization method to improve the preparative yield of the trans-isomer (T) as compared to any conventional process based on diastereomer separation without isomerization.

Based on our experimental results summarized in Table 1-4, it is predictable that the partial dynamic isomerization with a cis-selective TA may be conveniently carried out on a further selection of cis/trans-diastereomeric mixtures of 4-substituted cyclohexyl amines, such as 4-(2-hydroxyethyl)cyclohexan-1-amines (formula (IVa)+formula (Va)) and 4-((1,3-dioxolan-2-yl)methyl)cyclohexan-1-amines (formula (VIIa)+formula (VIIIa)).

Thus, it is expected that with purified soluble CvS_{W60C}-TA starting from a diastereomeric mixture of trans/cis-4-(2-hydroxyethyl)cyclohexan-1-amines (compound IVa+compound Va) in the presence of sub equimolar amount of sodium pyruvate the trans-isomer (compound IVa) can be obtained after several hours of reaction time in a good yield (exceeding the original amount of trans-isomer (IVa)) with high diastereomeric purity (deₜᵣₐₙₛ>90%) besides a moderate to small amount of ketone (compound VIa) in batch mode. It is also envisaged that with immobilized CvS_{W60C}-TA and sub equimolar amount of an amine acceptor ketone the trans-isomer (compound IVa) can be obtained from a of trans/cis-diastereomeric mixture of 4-(2-hydroxyethyl)cyclohexan-1-amines (compound IVa+compound Va) in high diastereomeric excess (deₜᵣₐₙₛ>95%) besides a moderate amount of ketone (compound VIa) by a continuous-flow mode process as well. It is expected that a major amount of the original cis-isomer (compound Va) content can be converted to the desired trans-isomer (compound IVa) in these dynamic isomerization processes.

It is also expected that the dynamic isomerization from a trans/cis-diastereomeric mixture of 4-((1,3-dioxolan-2-yl)methyl)cyclohexan-1-amines (compound VIIa+compound VIIIa) can result in several hours of reaction time with purified soluble CvS_{W60C}-TA a meaningfully enhanced amount of trans-isomer (compound VIIa) with high diastereomeric excess (deₜᵣₐₙₛ>90%) along with a modest amount of ketone (compound IXa) in batch mode. It is also foreseen that with immobilized CvS_{W60C}-TA and sub equimolar amount of an amine acceptor ketone the trans-isomer (compound VIIa) can be obtained from a of trans/cis-diastereomeric mixture of 4-((1,3-dioxolan-2-yl)methyl)cyclohexan-1-amines (compound VIIa+compound VIIIa) in high diastereomeric excess (deₜᵣₐₙₛ>95%) besides a modest amount of ketone (compound VIa) by a continuous-flow mode process as well. It is predicted that a significant amount of the original cis-isomer (compound VIIIa) content can be converted to the desired trans-isomer (compound VIIa) in these dynamic isomerization processes.

Thus, we have developed for the first time a biocatalytic route for the dynamic isomerization leading to 2-(trans-4-aminocyclohexyl)acetic acid alkyl esters [(formula Ia-d), particularly 2-(trans-4-aminocyclohexyl)acetic acid ethyl ester (formula Ib) or 2-(trans-4-aminocyclohexyl)acetic acid isopropyl ester (formula Id), most preferably 2-(trans-4-aminocyclohexyl)acetic acid ethyl ester (formula Ib)], as key intermediate of Cariprazine synthesis. It means an alternative and also an industrially applicable approach for the preparation of 2-(trans-4-aminocyclohexyl)acetic acid ethyl ester HCl (formula IIb·HCl) instead of recrystallization.

In a similar way, trans-4-(2-hydroxyethyl)cyclohexan-1-amine of formula (IVa) can also be prepared from a cis/trans-diastereomeric mixture of 4-(2-hydroxyethyl)cyclohexan-1-amines (formula (IVa)+formula (Va)) with a single transaminase biocatalyst in whole-cell, soluble or immobilized form in the presence of an amine acceptor used in sub-equimolar up to equimolar quantities in batch mode.

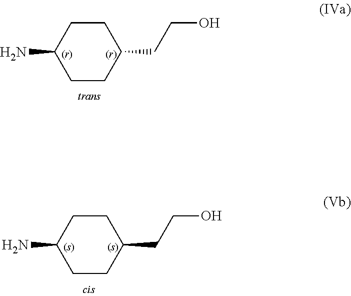 [SMILES: N[C@H]1CC[C@H](CCO)CC1 ; N[C@H]1CC[C@@H](CCO)CC1]

Lastly, the single transaminase-catalyzed dynamic isomerization enables the conversion of a cis/trans-diastereomeric mixture of 4-((1,3-dioxolan-2-yl)methyl)cyclohexan-1-amines (formula (VIIa)+formula (VIIIa)) to trans-4-((1,3-dioxolan-2-yl)methyl)cyclohexan-1-amine of formula (VIIa) using the transaminase biocatalyst in whole-cell, soluble or immobilized form in the presence of an amine acceptor in sub-equimolar up to equimolar quantities in batch mode.

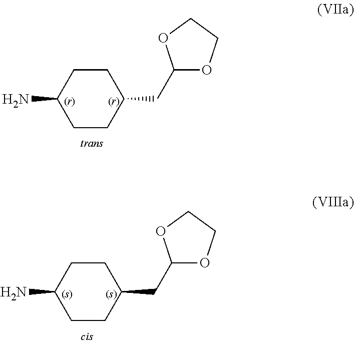 [SMILES: N[C@H]1CC[C@H](CC2OCCO2)CC1 ; N[C@H]1CC[C@@H](CC2OCCO2)CC1]

In summary, the process according to the present invention has several advantages and benefits.

The invented process
makes it possible to convert a predominantly large proportion of the cis by-product (formula C, particularly formulas II, V, or VIII).
is much more efficient than the separation processes that have been part of the traditional manufacturing process.
can be carried out not only in a batch mode, but also in a continuous flow mode.
that was developed on a laboratory scale (<gram) is also industrially feasible, the developed procedure can be scaled up.
can also be performed with a native enzyme.
requires only a single transaminase, while the literature for dynamic separation/transformation of enantiomers mostly uses two TAs.
represents a significant advantage in terms of efficiency when using the 4-substituted cyclohexanone (formula K) as the amine acceptor in the process carried out with the cis/trans-amine mixture (formulas C+T)), because there is no need to introduce other substances in addition to the intermediate ketone (formula K, particularly formulas III, VI, or IX).
can also open new possibilities during the period of generic production of Cariprazine.
with a single transaminase-catalyzed dynamic isomerization is applicable for the synthesis of further trans-4-substituted cyclohexyl amines, such as trans-4-(2-hydroxyethyl)cyclohexan-1-amine of formula (IVa) or trans-4-((1,3-dioxolan-2-yl)methyl)cyclohexan-1-amine of formula (VIIa) which may serve as alternative intermediates for the preparation of Cariprazine.

Based on the details and findings described above, in general, the present invention relates to the process to produce (1r,4r)-4-substituted cyclohexane-1-amine [=trans-4-substituted cyclohexane-1-amine] of formula (T) starting from a diastereomeric mixture of 4-substituted cyclohexane-1-amines (formula (C)+formula (T))

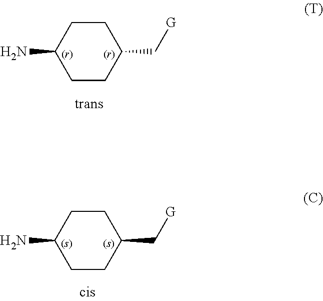

or any salt of them, where in formula (T) and in formula (C) G represents a substituent, selected from
a hydrogen atom;
a C₁₋₆ alkyl group;
an ester moiety (—COOR), where R represents a suitable alkyl, aralkyl or aryl group, preferably a C₁₋₆ alkyl group, more preferably a substituent selected from methyl, ethyl, propyl and isopropyl group;
a CH₂—OR′ group, where R′ represents hydrogen atom, or a hydroxyl protecting group;
a protected aldehyde group of formula

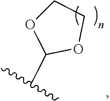

where n is an integer of 1 to 2;
a substituted or unsubstituted aryl group, preferably phenyl group; or
an aralkyl group, preferably benzyl group
   in such a way that the diastereomeric mixture is reacted with a single transaminase biocatalyst in whole-cell, soluble or immobilized form in the presence of an amine acceptor used in sub-equimolar up to equimolar quantities.

According to a particular embodiment of the present invention this general production process can be carried out in batch mode or in continuous-flow mode.

According to a preferred embodiment of the present invention this general production process can be carried out starting from diastereomeric mixture of the 4-substituted cyclohexane-1-amines (formula (C)+formula (T)) is in free base form.

According to another preferred embodiment of the present invention this general production process can be carried out starting from diastereomeric mixture of 4-substituted cyclohexane-1-amines (formula (C)+formula (T)) is in salt form, preferably in hydrochloride salt form (formula (C·HCl)+formula (T·HCl)).

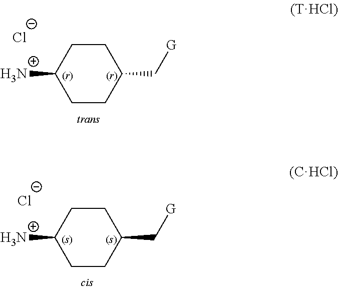

According to another preferred embodiment of the present invention this general production process can be carried out starting from a diastereomeric mixture of 4-substituted cyclohexane-1-amines (formula (C)+formula (T)) or its salt form is provided as cis/trans isomers in a ratio from about 2:98 to about 99:1.

According to another preferred embodiment of the present invention in this general production process a transaminase comprising an amino acid sequence with at least about 37% sequence identity to Chromobacterium violaceum transaminase mutant (W60C) (CvS_{W60C}-TA: SEQ ID NO. 1) or to Vibrio fluvialis transaminase (VfS-TA: SEQ ID NO. 2) over a region of at least about 100 residues is used.

According to another preferred embodiment of the present invention in this general production process a transaminase comprising an amino acid sequence with at least about 40% sequence identity to Chromobacterium violaceum transaminase mutant (W60C) (CvS_{W60C}-TA: SEQ ID NO. 1) or to Vibrio fluvialis transaminase (VfS-TA: SEQ ID NO. 2) over a region of at least about 100 residues is used.

According to another preferred embodiment of the present invention in this general production process a transaminase comprising an amino acid sequence with at least about 50% sequence identity to Chromobacterium violaceum transaminase mutant (W60C) (CvS_{W60C}-TA: SEQ ID NO. 1) or to Vibrio fluvialis transaminase (VfS-TA: SEQ ID NO. 2) over a region of at least about 100 residues is used.

According to another preferred embodiment of the present invention in this general production process a transaminase comprising an amino acid sequence with at least about 60% sequence identity to Chromobacterium violaceum transaminase mutant (W60C) (CvS_{W60C}-TA: SEQ ID NO. 1) or to Vibrio fluvialis transaminase (VfS-TA: SEQ ID NO. 2) over a region of at least about 100 residues is used.

According to another preferred embodiment of the present invention in this general production process a transaminase comprising an amino acid sequence with at least about 75% sequence identity to Chromobacterium violaceum transaminase mutant (W60C) (CvS_{W60C}-TA: SEQ ID NO. 1) or to Vibrio fluvialis transaminase (VfS-TA: SEQ ID NO. 2) over a region of at least about 100 residues is used.

According to another preferred embodiment of the present invention in this general production process a transaminase comprising an amino acid sequence with at least about 90% sequence identity to Chromobacterium violaceum transaminase mutant (W60C) (CvS_{W60C}-TA: SEQ ID NO. 1) or to Vibrio fluvialis transaminase (VfS-TA: SEQ ID NO. 2) over a region of at least about 100 residues is used.

According to a particular embodiment of the present invention in this general production process a suitable ketone or aldehyde is used as amin acceptor compound in sub-equimolar amounts.

According to a preferred embodiment of the present invention in this general production process a 4-substituted cyclohexanone of formula K

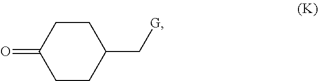

wherein G is as described in Claim 1 for the formula (C) and formula(T), is used as amine acceptor ketone.

As for the first aspect thereof, the present invention relates to the process where the starting diastereomeric mixture consists of 2-(4-aminocyclohexyl)acetic acid esters of formula (I) and formula (II)

| 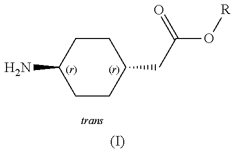 | |
|---|---|
| 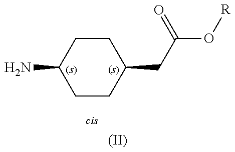 | |
| (I, II) | R |
| a | Me |
| b | Et |
| c | Pr |
| d | iPr, |

where R represents a suitable alkyl, aralkyl or aryl group, preferably a C₁₋₆ alkyl group, more preferably a substituent selected from methyl, ethyl, propyl and isopropyl, in free base form or in salt form.

According to this preferred embodiment of the present invention sodium pyruvate is used as amine acceptor ketone in sub-equimolar amounts.

According to this preferred embodiment of the present invention 4-substituted cyclohexanone of formula (III) is used as amine acceptor ketone

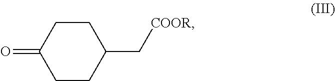

where R represents the same suitable alkyl, aralkyl or aryl group, preferably the same C₁₋₆ alkyl group, more preferably the substituent selected from methyl, ethyl, propyl and isopropyl as defined for formulas (I) and (II).

According to this preferred embodiment of the present invention ethyl 2-(4-oxocyclohexyl)acetate of formula (IIIb).

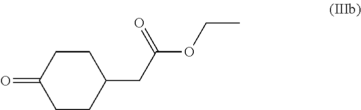

is used as amine acceptor ketone.

According to this preferred embodiment of the present invention isopropyl 2-(4-oxocyclohexyl)acetate of formula (IIId)

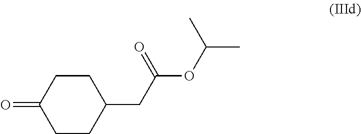

is used as amine acceptor ketone.

According to this preferred embodiment of the present invention the Chromobacterium violaceum mutant (W60C) enzyme/CvS_{W60C}-TA, characterized by SEQ ID NO 1/is used as transaminase in batch mode.

According to this preferred embodiment of the present invention the Chromobacterium violaceum mutant (W60C) transaminase/CvS_{W60C}-TA, characterized by SEQ ID NO 1/is used in whole-cell form, or in immobilized whole-cell form, or in soluble cell-free form, or in immobilized cell-free form.

According to this preferred embodiment of the present invention the Vibrio fluvialis enzyme/VfS-TA, characterized by SEQ ID NO 2/is used as transaminase in batch mode.

According to this preferred embodiment of the present invention the Vibrio fluvialis transaminase/VfS-TA, characterized by SEQ ID NO 2/is used in whole-cell form, or in immobilized whole-cell form, or in soluble cell-free form, or in immobilized cell-free form.

According to this preferred embodiment of the present invention a cis-selective Chromobacterium violaceum transaminase mutant (W60C)/CvS_{W60C}-TA/is used in continuous-flow mode.

According to this preferred embodiment of the present invention a cis-selective Chromobacterium violaceum transaminase mutant (W60C)/CvS_{W60C}-TA/with covalent immobilization onto a porous polymer support is used.

According to this preferred embodiment of the present invention starting from a diastereomeric mixture of 2-(4-aminocyclohexyl)acetic acid ethyl ester hydrochloride salt (formula Ib·HCl+formula IIb·HCl) pure 2-(trans-4-aminocyclohexyl)acetic ethyl ester (formula Ib) is produced.

According to this preferred embodiment of the present invention starting from a diastereomeric mixture of 2-(4-aminocyclohexyl)acetic acid isopropyl ester hydrochloride salt (formula Id·HCl+formula IId·HCl) pure 2-(trans-4-aminocyclohexyl)acetic isopropyl ester (formula Id) is produced.

According to a preferred embodiment of the present invention the production process of a 2-(trans-4-aminocyclohexyl)acetic acid ester (I), preferably a C₁₋₆ alkyl ester, starting from a cis/trans-diastereomeric mixture of 2-(4-aminocyclohexyl)acetic acid ester (I+II), preferably C₁₋₆ alkyl esters, either in free base form of amine or amine form liberated from hydrochloride salt form can be carried out in batch mode with a whole-cell, partially or fully purified soluble, or an immobilized form of a cis-selective transaminase (preferably W60C mutant of the TA from Chromobacterium violaceum/CvS_{W60C}-TA/or TA form Vibrio fluvialis/VfS-TA/), or in a continuous-flow mode with an immobilized form of the same cis-selective transaminases (CvS_{W60C}-TA or VfS-TA) in the presence of an amine acceptor used in sub-equimolar quantities.

According to the most preferred embodiment of the present invention 2-(trans-4-aminocyclohexyl)acetic acid ethyl ester product (formula Ib) is used in the manufacture of trans-N-{4-[2-[4-(2,3-dichlorophenyl)piperazin-1-yl]ethyl]cyclohexyl}-N′,N′-dimethylurea, commonly known as Cariprazine.

According to a preferred embodiment of the first aspect of present invention the production process of a 2-(trans-4-aminocyclohexyl)acetic acid ester (I) starting from a cis/trans-diastereomeric mixture of 2-(4-aminocyclohexyl)acetic acid ester (I+II) either in free base form of amine or amine form liberated from hydrochloride salt form can be carried out in batch mode with a whole-cell,-partially or fully purified soluble, or an immobilized form of a cis-selective transaminase in the presence of an amine acceptor used in sub-equimolar quantities.

According to a particular embodiment of the first aspect of present invention the production process of a 2-(trans-4-aminocyclohexyl)acetic acid ester (I) can be carried out starting from a cis/trans-diastereomeric mixture of 2-(4-aminocyclohexyl)acetic acid ester (I+II) in free base form of amine.

According to another particular embodiment of the first aspect of present invention the production process of a 2-(trans-4-aminocyclohexyl)acetic acid ester (I) can be carried out starting from a cis/trans-diastereomeric mixture of 2-(4-aminocyclohexyl)acetic acid esters (I+II) in amine form liberated from a salt form, especially hydrochloride salt form.

According to another particular embodiment of the first aspect of present invention the production process of a 2-(trans-4-aminocyclohexyl)acetic acid ester (I) can be carried out by using a whole-cell,-partially or fully purified soluble, or an immobilized form of W60C mutant of the TA from Chromobacterium violaceum/CvS_{W60C}-TA/.

According to another particular embodiment of the first aspect of present invention the production process of a 2-(trans-4-aminocyclohexyl)acetic acid ester (I) can be carried out by using a whole-cell, partially or fully purified soluble, or an immobilized form of TA form Vibrio fluvialis/VfS-TA/).

According to a more preferred embodiment of the first aspect of present invention the production process of a 2-(trans-4-aminocyclohexyl)acetic acid C₁₋₆ alkyl ester can be carried out starting from a cis/trans-diastereomeric mixture of 2-(4-aminocyclohexyl)acetic acid C₁₋₆ alkyl esters either in free base form of amine or amine form liberated from hydrochloride salt form.

According to another more preferred embodiment of the first aspect of present invention the production process of a 2-(trans-4-aminocyclohexyl)acetic acid C₁₋₆ alkyl ester can be carried out starting from a cis/trans-diastereomeric mixture of 2-(4-aminocyclohexyl)acetic acid C₁₋₆ alkyl esters either in free base form of amine or amine form liberated from hydrochloride salt form where mixture of cis/trans isomers is provided in a ratio from about 2:98 to about 99:1.

According to another more preferred embodiment of the first aspect of present invention the production process of a 2-(trans-4-aminocyclohexyl)acetic acid C₁₋₆ alkyl ester can be carried out in the presence of a suitable ketone or aldehyde as amine acceptor used in sub-equimolar quantities.

According to another more preferred embodiment of the first aspect of present invention the production process of a 2-(trans-4-aminocyclohexyl)acetic acid C₁₋₆ alkyl ester can be carried out in the presence of sodium pyruvate as amine acceptor.

According to the most preferred embodiment of the first aspect of present invention the production process of trans-N-{4-[2-[4-(2,3-dichlorophenyl)piperazin-1-yl]ethyl]cyclohexyl}-N′,N′-dimethylurea, commonly known as Cariprazine, can be carried out starting from a cis/trans-diastereomeric mixture of 2-(4-aminocyclohexyl)acetic acid ethyl ester (formula Ib) either in free base form of amine or amine form liberated from hydrochloride salt form where mixture of cis/trans ester isomers is provided in a ratio from about 2:98 to about 99:1 with a whole-cell,-partially or fully purified soluble, or an immobilized form of a cis-selective transaminase and in the presence of 2-(4-oxocyclohexyl)acetic acid ester (formula III), most preferably ethyl 2-(4-oxocyclohexyl)acetate (formula IIIb) as amine acceptor used in sub-equimolar quantities.

According to another most preferred embodiment of the first aspect of present invention the production process of trans-N-{4-[2-[4-(2,3-dichlorophenyl)piperazin-1-yl]ethyl]cyclohexyl}-N′,N′-dimethylurea, commonly known as Cariprazine, can be conducted starting from a cis/trans-diastereomeric mixture of 2-(4-aminocyclohexyl)acetic acid ethyl ester (formula Ib) in batch reactor in a stepwise manner wherein
a. the mixture of trans/cis-diastereomeric mixture of 2-(4-aminocyclohexyl)acetic acid ethyl esters (formula (Ib)+formula (IIb)) is filled into a reactor operated in batch mode. in ratio of about 2:98 to about 99:1,
b. a transaminase having higher than 40% protein sequence identity to Chromobacterium violaceum transaminase mutant (W60C)/CvS_{W60C}-TA: SEQ ID NO. 1/or to Vibrio fluvialis transaminase/VfS-TA: SEQ ID NO. 2/in whole-cell form, or in immobilized whole-cell form, or in soluble cell-free form, or in immobilized cell-free form is added to the reactor in protein:[substrate (formula (Ib)+formula (IIb))] weight ratio of 1:10000 to 1:1,
c. a solution of a suitable ketone used as amine acceptor compound is added in sub-equimolar amount to the mixture,
d. an acidic extraction purification step is applied to remove the forming ketone by product (formula (IIIb)),
e. the desired 2-(trans-4-aminocyclohexyl)acetic acid ethyl ester is extracted/separated/isolated as free amine (formula (Ib)) or as its salt (formula (Ib HA)) in a yield exceeding the proportion of the trans-isomer (formula (Ib) in the starting mixture.

As for the second aspect thereof, the present invention relates to the process where the starting diastereomeric mixture consists of 2-(4-aminocyclohexyl)ethan-1-ol derivatives of formula (IV) and formula (V)

| 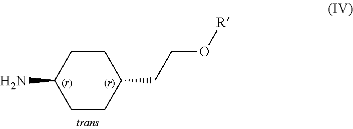 | |
|---|---|
| 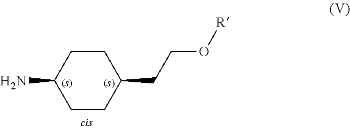 | |
| (IV, V) | R′ |
| a | H |
| b | CH₂Ph, |

where R′ represents a hydrogen atom, or suitable hydroxyl-protecting group, preferably a benzyl group, in free base form or in salt form.

According to this preferred embodiment of the present invention sodium pyruvate is used as amine acceptor ketone in sub-equimolar amounts.

According to this preferred embodiment of the present invention 4-substituted cyclohexanone of formula (VI) is used as amine acceptor ketone

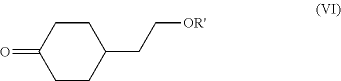

where R′ represents the same hydrogen atom, or suitable hydroxyl-protecting group, preferably a benzyl group, as defined for formulas (IV) and (V).

According to this preferred embodiment of the present invention 2-(4-oxocyclohexyl)ethan-1-ol of formula (VIa)

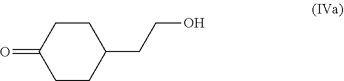

is used as amine acceptor ketone.

According to this preferred embodiment of the present invention the Chromobacterium violaceum mutant (W60C) enzyme/CvS_{W60C}-TA, characterized by SEQ ID NO 1/is used as transaminase in batch mode.

According to this preferred embodiment of the present invention the Chromobacterium violaceum mutant (W60C) transaminase/CvS_{W60C}-TA, characterized by SEQ ID NO 1/is used in whole-cell form, or in immobilized whole-cell form, or in soluble cell-free form, or in immobilized cell-free form.

According to this preferred embodiment of the present the Vibrio fluvialis enzyme/VfS-TA, characterized by SEQ ID NO 2/is used as transaminase in batch mode.

According to this preferred embodiment of the present invention the Vibrio fluvialis transaminase/VfS-TA, characterized by SEQ ID NO 2/is used in whole-cell form, or in immobilized whole-cell form, or in soluble cell-free form, or in immobilized cell-free form.

According to this preferred embodiment of the present invention a cis-selective Chromobacterium violaceum transaminase mutant (W60C)/CvS_{W60C}-TA/is used in continuous-flow mode.

According to this preferred embodiment of the present invention a cis-selective Chromobacterium violaceum transaminase mutant (W60C)/CvS_{W60C}-TA/with covalent immobilization onto a porous polymer support is used.

According to this preferred embodiment of the present invention starting from a diastereomeric mixture of 2-(4-aminocyclohexyl)ethan-1-ol hydrochloride salt (formula IVa·HCl+formula Va·HCl) pure 2-(trans-4-aminocyclohexyl)ethan-1-ol (formula IVa) is produced.

As for the third aspect thereof, the present invention relates to the process where the starting diastereomeric mixture consists of 2-(4-aminocyclohexyl)acetaldehyde derivatives of formula (VII) and formula (VIII)

| (VII, VIII) | n |
|---|---|
| a | 1 |
| b | 2 |

, where n is an integer of 1 to 2.

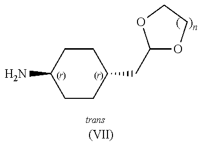

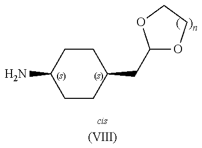

where n is an integer of 1 to 2.

According to this preferred embodiment of the present invention a sodium pyruvate is used as amine acceptor ketone in sub-equimolar amounts.

According to this preferred embodiment of the present invention a 4-substituted cyclohexanone of formula (IX) is used as amine acceptor ketone

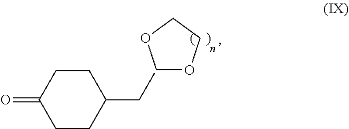

where n represents the same integer, as defined for formulas (VII) and (VIII).

According to this preferred embodiment of the present invention the Chromobacterium violaceum mutant (W60C) enzyme/CvS_{W60C}-TA, characterized by SEQ ID NO 1/is used as transaminase in batch mode.

According to this preferred embodiment of the present invention the Chromobacterium violaceum mutant (W60C) transaminase/CvS_{W60C}-TA, characterized by SEQ ID NO 1/is used in whole-cell form, or in immobilized whole-cell form, or in soluble cell-free form, or in immobilized cell-free form.

According to this preferred embodiment of the present invention the Vibrio fluvialis enzyme/VfS-TA, characterized by SEQ ID NO 2/is used as transaminase in batch mode.

According to this preferred embodiment of the present invention the Vibrio fluvialis transaminase/VfS-TA, characterized by SEQ ID NO 2/is used in whole-cell form, or in immobilized whole-cell form, or in soluble cell-free form, or in immobilized cell-free form.

According to this preferred embodiment of the present invention a cis-selective Chromobacterium violaceum transaminase mutant (W60C)/CvS_{W60C}-TA/is used in continuous-flow mode.

According to this preferred embodiment of the present invention a cis-selective Chromobacterium violaceum transaminase mutant (W60C)/CvS_{W60C}-TA/with covalent immobilization onto a porous polymer support is used.

According to this preferred embodiment of the present invention starting from a diastereomeric mixture of 4-((1,3-dioxolan-2-yl)methyl)cyclohexan-1-amines (formula VIIa+formula VIIIa) pure trans-4-((1,3-dioxolan-2-yl)methyl)cyclohexan-1-amine (formula VIIa) is produced.

The invention is illustrated by the following non-limiting examples.

### EXAMPLES

### Materials

Except otherwise not stated, all solvents and chemicals were purchased from the following commercial suppliers: Sigma Aldrich (Saint Louis, MO, USA), Alfa Aesar Europe (Karlsruhe, Germany), Merck (Darmstadt, Germany), Fluka (Milwaukee, WI, USA) and used without further purification. MAT540 (MATSPHERE™ SERIES 540—hollow silica microspheres etched with aminoalkyl and vinyl functions, with an average particle diameter of 10 μm) was obtained from Materium Innovations (Granby, QC, Canada). Ethyleneamine-functionalized methacrylic polymer resins (ReliZyme™ EA403/S; polymethyl methacrylate supports, particle size 150-300 μm, pore size 400-600 A) and epoxide-functionalized methacrylic polymer resins (ReliZyme™ EP403/S; polymethyl methacrylate supports, particle size 150-300 μm, pore size 400-600 A) were purchased from Resindion S.r.L. (Binasco, Italy).

Samples of 2-(4-aminocyclohexyl)acetic acid hydrochloride as cis/trans diastereomeric mixture [T·HCl+C·HCl (G=COOH)] and the cis-diastereomer of 2-(4-aminocyclohexyl)acetic acid ethyl ester hydrochloride (IIb·HCl with de˜90.2%) were obtained from the industrial scale production process according to WO2010/070368. The tert-butyl N-[4-(2-hydroxyethyl)cyclohexyl]carbamate can be prepared as disclosed by Wu Y.-J., et al (WO2018081384 A1 (2018)).

### Analytical Methods

### Thin Layer Chromatography

TLC was carried out using Kieselgel 60 F254 (Merck) sheets. Spots were visualized under UV light (Vilber Lourmat VL-6.LC, 254 nm) or after treatment with 5% ethanolic phosphomolybdic acid solution or 3% isopropanol ninhydrin solution and heating of the dried plates.

### Infrared Spectroscopy

Infrared spectra were recorded on a Bruker ALPHA FT-IR spectrometer and wavenumbers of bands are listed in cm⁻¹.

### Gas Chromatography

The reaction mixtures of TA-catalyzed reductive amination of ketones (general formulas K, III, VI, and IX) or dynamic isomerization of diastereomeric mixture of 4-substituted cyclohexane-1-amines (general formulas, C+T, I+II, IV+V, and VII+VIII) were analyzed—after derivatization of the amines to the corresponding acetamides by treatment of an excess acetic anhydride in ethyl acetate solution—on an Agilent 5890 GC (Santa Clara, USA) equipped with flame ionization detector (FID) using a non-polar HP-5 column [Agilent J&W; 30 m×0.25 mm×0.25 μm film thickness of (5%-Phenyl)methylpolysiloxane] or an Agilent 4890 GC equipped with a chiral Hydrodex β-6 TBDM column (Macherey-Nagel; 25 m×0.25 mm×0.25 μm film thickness of heptakis-(2,3-di-O-methyl-6-O-t-butyl-dimethylsilyl)-β-cyclodextrin) using H₂ carrier gas (injector: 250° C., detector: 250° C., head pressure: 12 psi, split ratio: 50:1). Temperature programs: TP1: 180-210° C. with 5° C./min, 210° C. for 4 min; TP2: 110-130° C. with 1° C./min, 130-180° C. with 20° C./min, 180° C. for 3 min.

### Retention Times:

1.45 min (compound IIIa), 2.91 min (acetamide of compound IIa), 3.11 min (acetamide of compound Ia) [on HP-5 column with TP1, molar response factor: (signals of acetamides of Ia+IIa/IIIa)=1.06];
1.95 min (compound IIIb), 3.93 min (acetamide of compound IIb), 4.11 min (acetamide of compound Ib) [on HP-5 column with TP1, molar response factor: (signals of acetamides of Ib+IIb/IIIb)=1.03];
1.58 min (compound IIId), 4.20 min (acetamide of compound IId), 4.40 min (acetamide of compound Id) [on HP-5 column with TP1, molar response factor: (signals of acetamides of Id+IId/IIId)=0.96];
1.69 min (compound VIa), 3.82 min (acetamide of compound IVa), 3.96 min (acetamide of compound Va) [on HP-5 column with TP1, molar response factor: (signals of acetamides of IVa+Va/VIa=1.05];
1.71 min [compound VI (R=Ac)], 3.81 min [acetamide of compound IV (R=Ac)], 3.94 min [acetamide of compound V (R=Ac)][on HP-5 column with TP1, molar response factor (signals of acetamides of IV+V (R=Ac)/VI (R=Ac)=1.03];
1.91 min (compound IXa), 4.45 min (acetamide of compound VIIa), 4.60 min (acetamide of compound VIIIa) [on HP-5 column with TP1, molar response factor (signals of acetamides of VIIa+VIIIa/IXa)=1.05];
2.28 min [compound IX (n=2)], 5.49 min [acetamide of compound VII (n=2)], 5.65 min [acetamide of compound VIII (n=2)][on HP-5 column with TP1, molar response factor (signals of acetamides of VII+VIII (n=2)/IX (n=2)=1.03];
3.45 min (compound K (G=H)), 19.72 min (acetamide of compound T (G=H)), 20.29 min (acetamide of compound C (G=H)) [on Hydrodex column with TP2, molar response factor (acetamides of (C+T (G=H))/(K (G=H))=1.90];
5.99 min (compound K (G=Me)), 22.64 min (acetamide of compound C (G=Me)), 22.85 min (acetamide of compound T (G=Me)) [on Hydrodex column with TP2, molar response factor (acetamides of (C+T (G=Me))/(K (G=Me))=1.16];
2.32 min (compound K (G=Ph)), 5.17 min (acetamide of compound C (G=Ph)), 5.51 min (acetamide of compound T (G=Ph)) [on HP-5 column with TP1, molar response factor (acetamides of (C+T (G=Ph))/(K (G=Ph))=1.06];
2.74 min (compound K (G=CH₂Ph)), 6.24 min (acetamide of compound C (G=CH₂Ph)), 6.56 min (acetamide of compound T (G=CH₂Ph)) [on HP-5 column with TP1, molar response factor (acetamides of (C+T (G=CH₂Ph))/(K (G=CH₂Ph))=0.89];

### Mass Spectroscopy

HRMS and MS-MS analyses were performed on a Thermo Velos Pro Orbitrap Elite (Thermo Fisher Scientific) system. The ionization method was ESI operated in positive ion mode. The protonated molecular ion peaks were fragmented by CID at a normalized collision energy of 35%. For the CID experiment helium was used as the collision gas. The samples were dissolved in methanol. Data acquisition and analysis were accomplished with Xcalibur software version 2.0 (Thermo Fisher Scientific).

### Nuclear Magnetic Resonance Spectroscopy

All NMR samples were dissolved in DMSO-d₆ solvent and the spectra were acquired in standard 5-mm tubes at 25° C. on either of the following Avance III HDX spectrometers from Bruker BioSpin GmbH, Rheinstetten, Germany (proton frequencies are given): 400 MHz (with 1H-19F/15N-31P Prodigy CryoProbe and a SampleCase sample changer), 500 MHz (500 S2 1H/13C/15N TCI Extended Temperature CryoProbe) or 800 MHz (800 SA 1H&19F/13C/15N TCI CryoProbe).

### General Procedure for Synthesis of (4-Alkoxycarbonylmethyl)Cyclohexanone (III)

Solution of the previously hexane-washed sodium hydride (1.7 eq.) in dry tetrahydrofuran (50 mL) was cooled to (−5)-0° C. Holding the temperature at 0-5° C. the solution of the corresponding phosphonate (1.2 eq. of ethyl 2-(diethoxyphosphoryl)acetate or isopropyl 2-(diisopropoxyphosphoryl)acetate) in dry tetrahydrofuran (50 mL) was added and the resulted mixture was stirred at 0° C. for 0.5 h and at room temperature for 1 h. After cooling again to (−5)-0° C. a solution of 1,4-cyclohexanedione mono ethylene ketal (1 eq.: −80 mmol) in dry THF (50 mL) was added dropwise, and the resulting mixture was stirred at 0° C. for 1 h then at room temperature overnight. The THF was evaporated from the reaction mixture and the residue was diluted with brine (60 mL) and the aqueous phase was extracted with ethyl acetate (3×80 mL). The unified organic phases were washed with saturated brine (80 mL) and dried over Na₂SO₄ and concentrated in vacuum to yield the crude alkyl 2-(1,4-dioxaspiro[4,5]decan-8-ylidene)acetate.

Without further purification, the unsaturated crude alkyl 2-(1,4-dioxaspiro[4,5]decan-8-ylidene)acetate was hydrogenated. After dissolving in the corresponding alcohol (50-200 mL), the solution was treated with 10% Pd/C (10w/w %) under 1 bar of hydrogen until the hydrogenation was complete (followed by TLC, eluent: hexane:EtOAc=2:1). After completion of the reaction, the mixture was filtered through Celite© and the solvent was removed by vacuum rotary evaporation to yield the saturated alkyl 2-(1,4-dioxaspiro[4,5]decan-8-yl)acetate.

To remove the ketone protecting group, the alkyl 2-(1,4-dioxaspiro[4,5]decan-8-yl)acetate (1 eq.) were dissolved in the corresponding alcohol (100-150 mL) and cooled to 0° C. IN HCl (3 eq.) solution was added dropwise and stirred at 0° C. for 1 h than at RT overnight. After the reaction was complete, it was cooled to 0° C. and the pH was adjusted to pH 7 by 1N NaOH.

The mixture was extracted with ethyl acetate (3×80 mL) and the unified organic phases were extracted with saturated brine (80 mL) and dried over Na₂SO₄ and concentrated in vacuum. The crude product was purified by silica gel column chromatography (eluent: hexane-EtOAc=4:1) to give (4-alkoxycarbonylmethyl)cyclohexanone (characterized with formula III).

### Ethyl 2-(4-oxocyclohexyl)acetate (IIIb)

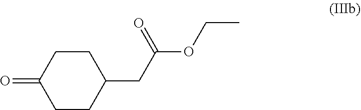

According to the general description, reaction of the solution of ethyl 2-(diethoxyphosphoryl)acetate (18.3 ml, 20.7 g, 92.2 mmol) in dry THE (50 mL) and hexane-washed NaH (3.69 g, 154 mmol) in dry THF (40 mL) with 1,4-cyclohexanedione mono ethylene ketal (12.0 g, 76.8 mmol) in dry THF (50 mL) afforded ethyl 2-(1,4-dioxaspiro[4,5]decan-8-ylidene)acetate (16.8 g, 97% crude yield) as colorless liquid.

The reaction of ethyl 2-(1,4-dioxaspiro[4,5]decan-8-ylidene)acetate (16.7 g, 71.0 mmol) and 10% Pd/C (1.67 g) in ethanol (70 mL) under non-pressurized hydrogen atmosphere afforded ethyl 2-(1,4-dioxaspiro[4,5]decan-8-yl)acetate (16.5 g, 98% crude yield) as colorless oil.

Reaction of the ethyl 2-(1,4-dioxaspiro[4,5]decan-8-yl)acetate (15.0 g, 65.7 mmol) in ethanol (150 mL) with 1 N HCl (150 mL) afforded ethyl-2-(4-oxocyclohexyl)acetate (formula IIIb, 5.32 g, 42% purified yield) as a colorless oil.

¹H NMR (500 MHz, DMSO-d₆) δ_{H}: 4.07 (2H, q, J=7.1 Hz, OCH₂—CH₃), 2.39 (2H, td, J=13.7 Hz, J=5.9 Hz, 2×CHₐₓ), 2.31 (2H, d, J=7.1 Hz, CH₂—COOEt), 2.21-2.15 (2H+1H, m, 2×CH_{eq}+CHₐₓ—CH₂COOEt), 1.98-1.92 (2H, m, 2×CH_{eq}), 1.40 (2H, qd, J=12.1 Hz, J=4.3 Hz, 2×CHₐₓ), 1.19 (3H, t, J=7.1 Hz, CH₃—CH₂);

¹³C NMR (125 MHz, DMSO-d₆) δ_{C}: 210.4 (CO), 171.9 (CH₂—COOEt), 59.7 (OCH₂—CH₃), 39.8 (CH₂), 39.3 (CH₂), 32.3 (CH—CH₂COOEt), 31.5 (CH₂), 14.0 (CH₃);

ESI-HRMS: M+H=185.11727 (delta=0.3 ppm; C₁₀H₁₇O₃). HR-ESI-MS-MS (CID=35%; rel. int. %): 167(60) and 139(100).

IR (neat) νₘₐₓ: 2933, 1710, 1449, 1368, 1345, 1278, 1201, 1150, 1094, 1029, 968, 754, 503 cm⁻¹.

GC (HP 5 column): t_{R}=1.95 min.

### Isopropyl-2-(4-oxocyclohexyl)acetate (IIId)

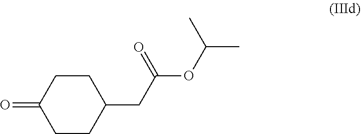

According to the general description, reaction of the isopropyl 2-(diisopropoxyphosphoryl)acetate (25.00 g, 93.3 mmol) with hexane-washed NaH (3.75 g, 156.4 mmol) in dry THF (100 mL) with 1,4-cyclohexanedione mono ethylene ketal (12.2 g, 78.2 mmol) in dry THE (50 mL) afforded isopropyl 2-(1,4-dioxaspiro[4,5]decan-8-ylidene)acetate (17.1 g, 91% crude yield) as colorless liquid.

The reaction of isopropyl 2-(1,4-dioxaspiro[4,5]decan-8-ylidene)acetate (15.00 g, 62.46 mmol) and 10% Pd/C (1.5 g) in isopropanol (220 mL) under non-pressurized hydrogen atmosphere afforded isopropyl-2-(1,4-dioxaspiro[4,5]decan-8-yl)acetate (14.6 g, 97% crude yield) as colorless oil.

Reaction of the isopropyl 2-(1,4-dioxaspiro[4,5]decan-8-yl)acetate (14.00 g, 57.85 mmol) in isopropanol (170 mL) and 1 N HCl (170 mL) afforded ethyl-2-(4-oxocyclohexyl)acetate (formula IIId, 8.47 g, 74% purified yield) as a colorless oil.

¹H NMR (500 MHz, DMSO-d₆) δ_{H}: 4.91 (1H, quint, J=6.3 Hz, CH—(CH₃)₂), 2.39 (2H, td, J=13.8 Hz, J=6.0 Hz, 2×CHₐₓ), 2.28-2.27 (2H, m, CH₂), 2.19-2.14 (1H+2H, m, CHₐₓ—CH₂COOⁱPr, 2×CH_{eq}), 1.96-1.92 (2H, m, 2×CH_{eq}), 1.40 (2H, qd, J=13.0 Hz, J=4.1 Hz, 2×CHₐₓ), 1.19 (6H, d, J=6.3 Hz, 2×CH₃);

¹³C NMR (125 MHz, DMSO-d₆) δ_{C}: 210.4 (CO), 171.4 (COOⁱPr), 66.9 (CH—(CH₃)₂), 39.6 (CH₂—COOⁱPr), 32.3 (CH—CH₂COOⁱPr)+CH₂), 31.4 (CH₂), 21.5 (CH₃);

HRMS: M+H=199.13276 (delta=−0.6 ppm; C₁₁H₁₉O₃). HR-ESI-MS-MS (CID=35%; rel. int. %): 181(5); 171(11); 167(100); 157(62); 153(91) and 139(64);

IR (neat) νₘₐₓ: 2979, 1711, 1449, 1374, 1278, 1203, 1161, 1107, 967 cm⁻¹.

GC (HP 5 column): t_{R}=1.58 min.

24

### cis/trans-Diastereomeric mixtures of 4-substituted cyclohexan-1-aminium chlorides (compounds I·HCl+II·HCl or compounds C·HCl+T·HCl)

### 4-(2-Methoxy-2-oxoethyl)cyclohexan-1-aminium chloride (compounds Ia·HCl+IIa·HCl)

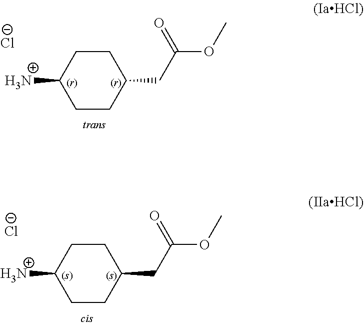

To a solution of 2-(4-aminocyclohexyl)acetic acid [cis/trans diastereomeric mixture, T·HCl+C·HCl (G=COOH)](1 g, 6.37 mmol) in methanol (60 mL) was added 5 M hydrochloric acid solution (9.56 mmol, 1.911 mL, 1.5 eq.). The reaction mixture was stirred at room temperature for 30 min (during this time, the initially opalescent solution cleared and TLC analysis (eluant:n-butanol:acetic acid:water=3:1:1; visulized by 3% ninhydrin in isopropanol; Rf_{acid}=0.62, Rf_{Me ester}=0.68) revealed complete conversion. Next, the solvent was removed using a rotary vacuum evaporator and the residue was dried in a vacuum drying chamber to yield the diastereomeric mixture of the desired methyl ester hydrochloride salt (compounds Ia·HCl+IIa·HCl, 1.28 g, 97% yield) as white solid.

IR (ATR) νₘₐₓ: 2934, 2895, 2863, 1732, 1610, 1507, 1458, 1437, 1365, 1295, 1226, 1168, 1132, 1018 cm⁻¹.

### (1s,4s)-4-(2-Methoxy-2-oxoethyl)cyclohexan-1-aminium chloride (cis-compound IIa·HCl)

¹H NMR (500 MHz, DMSO-d₆) δ_{H}: 8.12 (3H, br, NH₃⁺), 3.58 (3H, s, OCH₃), 3.29 (1H, m, CHₑₖᵥ—NH₃⁺), 2.33 (2H, d, J=7.5 Hz, CH₂COOMe), 1.95 (1H, m, CH_{eq}—CH₂COOMe), 1.81-1.74 (2H, m, 2×CH), 1.70-1.58 (4H, m, 4×CH), 1.46-1.35 (2H, m, 2×CH).

¹³C NMR (125 MHz, DMSO-d₆) δ_{C}: 176.39 (COO), 52.14 (OCH₃), 48.53 (NCH), 38.15 (CH₂), 30.65 (CH), 26.23 (2×CH₂), 26.06 (2×CH₂).

### (1r,4r)-4-(2-Methoxy-2-oxoethyl)cyclohexan-1-aminium chloride (trans-compound Ia·HCl)

¹H NMR (500 MHz, DMSO-d₆) δ_{H}: 8.12 (3H, br, NH₃⁺), 3.59 (3H, s, OCH₃), 3.04 (1H, m, CHₐₓ—NH₃⁺), 2.21 (2H, d, J=7.6 Hz, CH₂COOMe), 1.98-1.86 (2H, m, 2×CH_{eq}), 1.75 (1H, m, CHₐₓ—CH₂COOMe), 1.72 (2H, br d, J=14.0 Hz, CH_{eq}CHNH₃⁺), 1.46-1.35 (2H, m, 2×CH), 1.02 (2H, qd, J=12 Hz, J=4 Hz, 2×CHₐₓ)

¹³C NMR (125 MHz, DMSO-d₆) δ_{C}: 176.43 (COO), 52.09 (OCH₃), 48.87 (NCH), 40.48 (CH₂), 33.01 (CH), 29.89 (2×CH₂), 29.85 (2×CH₂),

### 4-(2-Ethoxy-2-oxoethyl)cyclohexan-1-aminium chloride (compounds Ib·HCl+IIb·HCl)

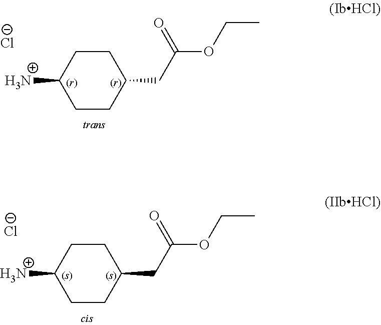

Reaction of ethyl 2-(4-oxocyclohexyl)acetate (IIIb, 1.50 g, 8.14 mmol) and 10% Pd/C (0.15 g) with ammonium formate (3.08 g, 48.8 mmol) in ethanol (40 mL) afforded the diastereomeric mixture of 4-(2-ethoxy-2-oxoethyl)cyclohexan-1-amine (compounds Ib+IIb, 1.24 g, 83% yield, cis/trans=2.30:1.00 (¹H-NMR)) as colorless oil. Lastly after the introducing of HCl-gas 4-(2-ethoxy-2-oxoethyl)cyclohexan-1-aminium chloride (compounds Ib·HCl+IIb·HCl, 1.30 g, 72% yield) was formed as white solid.

### (1s,4s)-4-(2-Ethoxy-2-oxoethyl)cyclohexan-1-aminium chloride (cis-compound IIb·HCl)

¹H NMR (500 MHz, DMSO-d₆) δ_{H}: 8.14 (3H, br, NH₃⁺), 4.09-4.02 (2H, m, OCH₂), 3.18-3.09 (1H, m, CHₐₓ—NH₃⁺), 2.27 (2H, d, J=7.5 Hz, CH₂COOEt), 1.98-1.86 (1H, m, CH_{eq}—CH₂COOEt), 1.69-1.62 (4H, m, 4×CH), 1.53-1.43 (4H, m, 4×CH), 1.18 (3H, t, J=7.2 Hz, CH₃),

¹³C NMR (125 MHz, DMSO-d₆) δ_{C}: 171.96 (CO), 59.62 (OCH₂), 47.2 (CH—NH₃⁺), 37.92 (CH₂COOEt), 30.58 (CHₐₓ—CH₂COOEt), 25.96 (CH₂), 25.89 (CH₂), 14.04 (CH₃);

### (1r,4r)-4-(2-Ethoxy-2-oxoethyl)cyclohexan-1-aminium chloride (trans-compound Ib·HCl)

¹H NMR (500 MHz, DMSO-d₆) δ_{H}: 8.14 (3H, br, NH₃⁺), 4.09-4.02 (2H, m, OCH₂), 2.94-2.82 (1H, m, CHₐₓ—NH₃⁺), 2.18 (2H, d, J=7.6 Hz, CH₂COOEt), 1.98-1.86 (2×H, m, 2×CH_{eq}), 1.72 (2H, br d, J=14.0 Hz, CH_{eq}CHNH₃⁺), 1.64-1.55 (1H, m, CHₐₓ—CH₂COOEt), 1.34 (2H, qd, J=12.4 Hz, J=3.1 Hz, 2×CHₐₓ), 1.18 (3H, t, J=7.2 Hz, CH₃), 1.03 (2H, qd, J=12.7 Hz, J=3.5 Hz, 2×CHₐₓ)

¹³C NMR (125 MHz, DMSO-d₆) δ_{C}: 171.8 (CO), 59.6 (OCH₂), 48.9 (CH—NH₃⁺), 40.3 (CH₇₂COOEt), 33.1 (CHₐₓ—CH₂COOEt), 29.8 (CH₂), 29.75 (CH₂), 14.0 (CH₃);

HRMS: M+H=186.14853 (delta=−1.8 ppm; C₁₀H₂₀O₂N). HR-ESI-MS-MS (CID=35%; rel. int. %): 169(100); 141(2); 140(2); 123(9); 95(15) and 81(6);

IR (neat) νₘₐₓ: 2933, 2552, 2037, 1731, 1604, 1509, 1451, 1370, 1291, 1177, 1033 cm⁻¹.

### 4-(2-Isopropoxy-2-oxoethyl)cyclohexan-1-aminium chloride (compounds Id·HCl+IId·HCl)

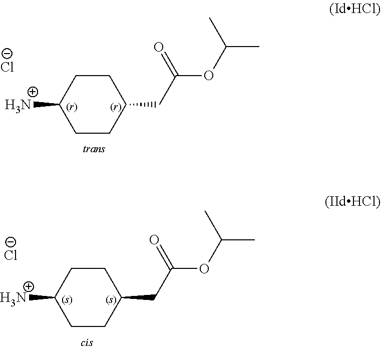

Reaction of isopropyl 2-(4-oxocyclohexyl)acetate (IIId, 2.00 g, 10.1 mmol) and 10% Pd/C (0.20 g) with ammonium formate (3.82 g, 60.5 mmol) in isopropanol (40 mL) afforded the diastereomeric mixture of 4-(2-isopropoxy-2-oxoethyl)cyclohexan-1-amine (compounds Id+IId, 1.71 g, 85% yield, cis/trans=1.07:1.00 (¹H-NMR)) as colorless oil. Lastly after the introducing of HCl-gas 4-(2-isopropoxy-2-oxoethyl)cyclohexan-1-aminium chloride (compounds Id·HCl+IId·HCl, 1.75 g, 73% yield) was formed as white solid.

### (1s,4s)-4-(2-Isopropoxy-2-oxoethyl)cyclohexan-1-aminium chloride (cis-compound Id·HCl)

¹H NMR (500 MHz, DMSO-d₆) δ_{H}: 8.10 (3H, br, NH₃⁺), 4.89 (1H, quint, J=6.25 Hz, CH—(CH₃)₂), 3.13 (1H, quint, J=5.6 Hz, CH_{eq}—NH₃⁺), 2.21 (2H, d, J=7.55 Hz, CH₂—COOⁱPr), 1.95-1.89 (1H, m, CHₐₓ—CH₂COOⁱPr), 1.67-1.64 (4H, m, 4×CH), 1.53-1.43 (4H, m, 4×CH), 1.18 (6H, d, J=1.71 Hz, 2×CH₃),

¹³C NMR (125 MHz, DMSO-d₆) δ_{C}: 171.5 (CO); 66.9 (CH—(CH₃)₂); 47.3 (CH—NH₃⁺), 38.2 (CH₂—COOⁱPr), 30.6 (CH—CH₂COOⁱPr), 26.0 (CH₂), 25.9 (CH₂), 21.5 (CH₃).

### (1r,4r)-4-(2-Isopropoxy-2-oxoethyl)cyclohexan-1-aminium chloride (trans-compound Id·HCl)

¹H NMR (500 MHz, DMSO-d₆) δ_{H}: 8.10 (3H, br, NH₃⁺), 4.88 (1H, quint, J=6.25 Hz, CH—(CH₃)₂), 2.88 (1H, tt, J=11.8 Hz, J=3.9 Hz, CHₐₓ—NH₃⁺), 2.14 (2H, d, J=6.96 Hz, CH₂—COOⁱPr), 1.95-1.89 (2H, m 2×CH_{eq}), 1.73-1.70 (2H, m, 2×CH), 1.62-1.55 (1H, CHₐₓ—CH₂COOⁱPr), 1.33 (2H, qd, J=12.7 Hz, J=3.2 Hz, 2×CHₐₓ), 1.17 (6H, d, J=1.75 Hz, 2×CH₃), 1.02 (2H, qd, J=12.8 Hz, J=3.2 Hz, 2×CH); ¹³C NMR (125 MHz, DMSO-d₆) δ_{C}: 171.3 (CO); 66.9 (CH—(CH₃)₂); 48.9 (CH—NH₃⁺), 40.6 (CH₂—COOⁱPr), 33.2 (CH—CH₂COOⁱPr), 29.8 (CH₂), 29.7 (CH₂), 21.5 (CH₃);

HRMS: M+H=200.16423 (delta=−1.4 ppm; C₁₁H₂₂O₂N). HR-ESI-MS-MS (CID=35%; rel. int. %): 183(5); 158(5); 141(100); 140(4); 123(6) and 81(8).

IR (neat) νₘₐₓ: 2944, 2627, 2553, 2056, 1729, 1607, 1510, 1458, 1391, 1297, 1182, 1107 cm⁻¹.

### 2-(4-Aminocyclohexyl)ethan-1-ol (compounds IVa+Va)

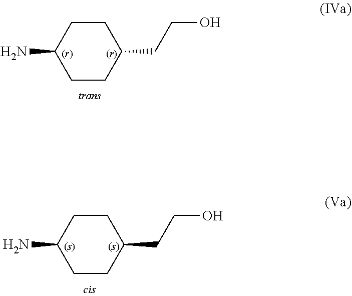 [SMILES: N[C@H]1CC[C@H](CCO)CC1 ; N[C@H]1CC[C@@H](CCO)CC1]

Into a round-bottomed flask were added NaBH₄ (173 mg, 4.5 mmol), tetrahydrofurane (THF, 15 mL) and cis/trans-2-(4-aminocyclohexyl)acetic acid hydrochloride [T·HCl+C·HCl (G=COOH)](300 mg, 1.91 mmol). To this mixture, a solution prepared from iodine (483 mg, 1.91 mmol) and THF (4.5 mL) was added dropwise at 0° C. (resulting in exothermic reaction with gas evolution) and the forming mixture was stirred under reflux for 24 h. After cooling to 0° C., methanol (8 mL) was added dropwise (resulting in heat and gas evolution and dissolving the formed white suspension). After evaporation of the solvent under vacuum, the residual crude product was purified by preparative thin layer chromatography (silica gel, dichloromethane:methanol=20:1 as eluant) to yield the diastereomeric mixture of alcohol IVa+Va (173.2 mg 63.6%, cis/trans˜46:54) as white powdery solid.

Melting point: 92° C.

¹H NMR (500 MHz, DMSO-d₆) δ_{H}: 3.54 (2H, t, J=5.4 Hz, OCH₂), 2.67 and 2.41 (1H, m, CH—N), 2.00 (1H, m); 1.71 (1H, m); 1.60-1.25 (6H, m); 1.25-1.15 (1H, m); 1.08 (TH, q); 0.89 (1H, q).

¹³C NMR (125 MHz, DMSO-d₆) δ_{C}: 62.31 and 62.19 (OCH₂), 58.67 and 56.46 (CHN), 40.69 (CHCH₂), 35.37 (CH), 33.55 and 33.40 (2×CH₂), 29.49 and 29.10 (2×CH₂).

IR (ATR) νₘₐₓ: 3483, 3455, 3259, 3227, 3141, 2925, 2888, 2877, 2856, 1598, 1454, 1445, 1356, 1327, 1164, 1050, 874 cm⁻¹.

### 4-(2-acetoxyethyl)cyclohexan-1-aminium chloride [compounds IV (R=Ac)+V (R=Ac)]

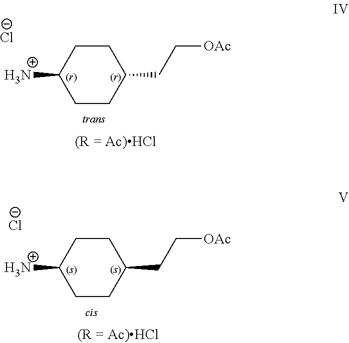 [SMILES: [Cl-] ; CC(=O)OCC[C@H]1CC[C@H]([NH3+])CC1 ; [Cl-] ; CC(=O)OCC[C@H]1CC[C@@H]([NH3+])CC1]

### tert-butyl N-[4-(2-acetoxyethyl)cyclohexyl]carbamate

To a solution of tert-butyl N-[4-(2-hydroxyethyl)cyclohexyl]carbamate [Wu Y.-J., et al (WO2018081384 A1 (2018)](0.4 g, 1.64 mmol), triethylamine (0.4 mL) and 4-dimethylaminopyridine (24 mg) in dichloromethane (15 mL), acetyl chloride (0.175 mL, 2.46 mmol) was added dropwise at 0° C., then the resulting mixture was stirred at room temperature for 4 h. After evaporation of the volatiles in vacuum, the residue was purified on a silica gel column using dichloromethane:methanol 20:1 eluent to yield the title product [compounds IV (R=Ac)+V (R=Ac)](0.39 g, 83%) as a substance that crystallizes in a refrigerator.

¹H NMR (500 MHz, CDCl₃) δ_{H}: 4.57 and 4.3 (1H, br, NH), 4.0 (2H, q, J=6.5 Hz, OCH₂), 3.64 and 3.28 (1H, br CHN), 1.97 (3H, s, COCH₃), 1.9 (1H, d, J=10 Hz), 1.7 (1H, d, J=11 Hz), 1.6-1.4 (5H, m), 1.38 (9H, s, 3×CH₃), 1.3-1.1 (2H, m), 1.05-0.9 (2H, m).

¹³C NMR (125 MHz, CDCl₃) δ_{C}: 171.25 (COCH₃), 155.27 (CONH), 79.12 (C), 62.66 (OCH₂), 49.87 and 46.52 (CHNH), 35.38 and 33.8 (CH₂), 33.3 (CH₂), 34.09 and 32.57 (CH), 31.69 (CH₂), 29.59 (CH₂), 28.44 (3×CH₃), 27.71 (CH₂), 21.02 (COCH₃).

### 4-(2-acetoxyethyl)cyclohexan-1-aminium chloride [compounds IV (R=Ac)·HCl+V (R=Ac)·HCl]

To the solution of tert-butyl N-[4-(2-acetoxyethyl)cyclohexyl]carbamate (0.39 g) in ethyl acetate (3.5 mL) was added a 20% solution of hydrochloric acid in ethyl acetate (2.4 mL), and the resulting mixture was stirred at room temperature for 2.5 h. Evaporation of the solvent under vacuum resulted in the title compounds (0.30 g, 100%).

¹H NMR (500 MHz, CDCl₃) δ_{H}: 8.33 and 4.60-4.10 (3H, br, NH₃′), 4.1 (2H, q, J=6.5 Hz, OCH₂), 3.47 and 3.12 (1H, br, CHN), 2.20 (1H, d, J=11.5 Hz), 2.06 and 2.05 (3H, s, COCH₃), 2.00-1.93 (1H, m), 1.88 (1H, d, J=13 Hz), 1.85-1.75 (1H, m), 1.72-1.5 (5H, m), 1.5-1.2 (1H, m), 1.03 (1H, q, J=13 Hz).

¹³C NMR (125 MHz, CDCl₃) δ_{C}: 171.22 (COCH₃), 62.40 and 62.21 (OCH₂), 50.95 and 48.73 (HNH), 35.06 (CH₂), 32.91 (CH₂), 33.26 and 31.50 (CH), 30.71 (CH₂), 30.68 (CH₂), 27.45 (CH₂), 26.42 (CH₂), 21.01 (COCH₃).

### 4-((1,3-Dioxolan-2-yl)methyl)cyclohexan-1-amine (compounds VIIa+VIIIa)

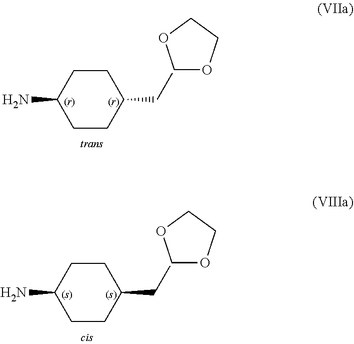

### tert-Butyl (4-(2-oxoethyl)cyclohexyl)carbamate

To a solution of tert-butyl [4-(2-hydroxyethyl)cyclohexyl]carbamate (0.2 g, 0.823 mmol) in dry dichloromethane (7 mL) was added pyridinium chlorochromate (PCC, 1.3 g) portionwise and the resulting mixture was stirred at room temperature for 1 h. The solvent was evaporated from the mixture under vacuum and the residue was purified by chromatography on silica gel column with dichloromethane to result the title compound (1.12 g, 58%) as a viscous oil that crystallized in the refrigerator.

¹H NMR (300 MHz, CDCl₃) 1H: 9.76 (1H, s, CHO), 4.64 and 4.4 (1H, br, NH), 3.72 and 3.36 (1H, br CHN), 2.43-2.3 (2H, dd, CH₂), 2.1-1.7 (2H, m), 1.71-1.55 (3H, m); 1.45 (9H, s, 3×CH₃), 1.0-1.35 (4H, m).

¹³C NMR (75 MHz, CDCl₃) δ_{C}: 202.2 (CHO), 155.4 (CONH), 79.2 (C—O), 50.7 and 49.6 (CHNH), 39.7 and 38.4 (CH₂), 33.2 (CH₂), 31.7 (CH₂), 30.5 (CH), 29.5 (CH₂), 28.45 (3×CH₃), 27.8 (CH₂).

tert-Butyl (4-((1,3-dioxolan-2-yl)methyl)cyclohexyl)carbamate [according to Bush-Petersen J., et al WO 2006050292A2 (2006)]

To a solution of tert-butyl (4-(2-oxoethyl)cyclohexyl)carbamate (0.61 g, 2.71 mmol) in acetonitrile (11.5 mL) were added oxalic acid·2H₂O (33 mg), MgSO₄ (0.5 g) and ethylene glycol (0.61 mL) and the mixture was stirred at room temperature for 18 h. After filtering the reaction mixture, the filtrate was diluted with ethyl acetate (40 mL) and washed with saturated NaHCO₃ solution (8 mL), water (8 mL) and brine (8 mL). After drying the organic phase over Na₂SO₄, the solvent was evaporated in vacuo to leave the title compound (0.59 g, 74%) as a heavy oil that crystallized in refrigerator (the sample contained ˜10% of tert-butyl [4-(2-hydroxyethyl)cyclohexyl]carbamate as impurity).

¹H NMR (500 MHz, CDCl₃) δ_{H}: 4.9 (1H, t, O—CH—O), 4.64 and 4.36 (1H, br, NH), 4.0-3.8 (4H, m, 2×CH₂), 3.70 and 3.36 (1H, br CHN), 2.1-1.8 (2H, m), 1.71-1.5 (5H, m), 1.45 (9H, s, 3×CH₃), 1.35-1.2 (2H, m), 1.0-1.2 (2H, m),

¹³C NMR (125 MHz, CDCl₃) δ_{C}: 155.4 (O═CNH), 103.6 and 103.4 (O—CH—O), 79.1 (C-0), 64.8 (2×OCH₂), 49.8 and 46.5 (CHNH), 40.7 and 33.3 (CH₂), 33.4 (CH₂), 32.1 (CH₂), 39.5 and 32.0 (CH), 29.7 (CH₂), 28.46 (3×CH₃), 28.2 (CH₂).

### 4-((1,3-Dioxolan-2-yl)methyl)cyclohexan-1-amine (compounds VIIa+VIIIa) [according to Bush-Petersen J., et al WO 2006050292A2 (2006)]

To a solution of tert-butyl (4-((1,3-dioxolan-2-yl)methyl)cyclohexyl)carbamate (0.57 g, 2.14 mmol) in ethyl acetate (5 mL) was added a 20% solution of hydrochloric acid in ethyl acetate (3.5 mL), and the resulting mixture was stirred at room temperature for 2 h. After evaporating the solvent under vacuum, the residual solid was dried in a vacuum chamber to give (0.45 g, 100%) as a solid powder. (This sample contained 28% of free aldehyde.) The solid was dissolved in ethylene glycol (0.52 mL) and the mixture was stirred at 40° C. for 8 h under reduced pressure (5 Hgmm). After diluting with ethyl acetate (40 mL), solid Na₂CO₃ (0.45 g) was added and the resulting mixture was stirred for a few minutes. After filtration, the organic phase was washed with water (2×10 mL) and brine (10 mL). After drying the organic phase over Na₂SO₄, the solvent was evaporated in vacuo to leave the title compound (0.16 g, 38%) as a viscous oil (the sample contained ˜7% of 2-(4-aminocyclohexyl)ethanal and ˜9% of 2-(4-aminocyclohexyl)ethan-1-ol as impurity).

The unified aqueous phases were extracted with dicloromethane (3×20 mL) and the resulting organic phase was dried over Na₂SO₄ and concentrated in vacuum to yield the title compound (23 mg, 6%) as a viscous oil (the sample contained ˜1.5% of 2-(4-aminocyclohexyl)ethanal and ˜5.5% of 2-(4-aminocyclohexyl)ethan-1-ol as impurity).

¹H NMR (500 MHz, CDCl₃) δ_{H}: 4.91 (1H, m, OCHO), 3.97 (2H, m, 2×OCH), 3.84 (2H, m, 2×OCH), 2.98 and 2.63 (1H, br, CHN), 2.19 (2H, br, NH₂), 1.91-1.79 (2H, m), 1.72-1.41 (5H, m), 1.35-1.21 (2H, m), 1.01-1.20 (2H, m).

¹³C NMR (125 MHz, CDCl₃) δ_{C}: 103.71 and 103.47 (OCHO), 64.69 (2×OCH₂), 50.56 (CHNH), 40.82 (CH₂), 33.23 (2×CH₂), 32.2 (2×CH₂), 31.99 (CH).

### 4-((1,3-Dioxan-2-yl)methyl)cyclohexan-1-amine [compounds VII (n=2)+VIII (n=2)]

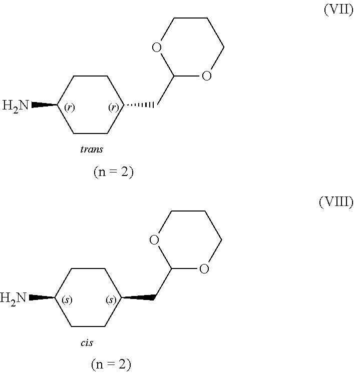 [SMILES: N[C@H]1CC[C@H](CC2OCCCO2)CC1 ; N[C@H]1CC[C@@H](CC2OCCCO2)CC1]

### tert-Butyl (4-((1,3-dioxan-2-yl)methyl)cyclohexyl)carbamate

To a solution of tert-butyl (4-(2-oxoethyl)cyclohexyl)carbamate (0.82 g, 3.4 mmol) in acetonitrile (15.5 mL) were added oxalic acid (44.4 mg), MgSO₄ (0.6 g) and propylene glycol (1.1 mL) and the mixture was stirred at room temperature for 18 h. After filtration, the filtrate was diluted with ethyl acetate (54 mL) and washed with saturated NaHCO₃ solution (11 mL), water (11 mL) and brine (22 mL). After drying the organic phase over Na₂SO₄, the solvent was evaporated in vacuo. The residue was purified on a silica gel column using dichloromethane:methanol 20:1 eluent to leave the title compound (0.93 g, 99%) as a heavy oil that crystallized in refrigerator (the sample contained ˜10% of tert-butyl [4-(2-hydroxyethyl)cyclohexyl]carbamate as impurity).

¹H NMR (500 MHz, CDCl₃) δ_{H}: 4.64 and 4.37 (1H, br, NH), 4.58 (1H, t, OCHO), 4.21-4.01 (2H, dd, J=4.5 Hz and 11.5 Hz, CH₂), 3.76 (2H, t, J=12.5 Hz), 3.71 and 3.36 (1H, br, CHN), 2.14-1.90 (2H, m), 1.79 (1H, d, J=11 Hz), 1.71-1.51 (5H, m), 1.51-1.46 (1H, m), 1.45 (9H, s, 3×CH₃), 1.35 (1H, d, J=13.5 Hz), 1.29-1.15 (1H, m), 1.15-1.01 (2H, m).

¹³C NMR (125 MHz, CDCl₃) δ_{C}: 155.28 (CONH), 101.05 and 100.92 (OCHO), 79.04 (C), 66.92 (2×OCH₂), 53.42 and 51.43 (CHNH), 42.04 and 32.37 (CH₂), 33.34 (CH₂), 33.13 and 31.60 (CH), 32.00 (CH₂), 29.61 (CH₂), 28.45 (3×CH₃), 28.13 (CH₂), 25.83 (CH₂).

### 4-((1,3-Dioxan-2-yl)methyl)cyclohexan-1-amine (compounds VII+VIII (n=2))

To a solution of tert-butyl (4-((1,3-dioxan-2-yl)methyl)cyclohexyl)carbamate (0.65 g, 2.18 mmol) in ethyl acetate (5.5 mL) was added a 20% solution of hydrochloric acid in ethyl acetate (4 mL), and the resulting mixture was stirred at room temperature for 1 h. After evaporating the solvent under vacuum, the residual solid was dried in a vacuum chamber to give (0.53 g, 100%) as a solid powder. (The sample contained 10% of free aldehyde.) The solid was dissolved in propylene glycol (0.6 mL) and the mixture was stirred at 40° C. for 8 h under reduced pressure (5 Hgmm). To the mixture diluted with ethyl acetate (40 mL) Na₂CO₃ (0.38 g) was added and the suspension was stirred for a few minutes. After filtration, the organic phase was washed with water (2×10 mL) and brine (10 mL). After drying the organic phase over Na₂SO₄, the solvent was evaporated in vacuo to leave the title compound (0.28 g, 60%) as a viscous oil (the sample contained ˜12.5% of 2-(4-aminocyclohexyl)ethan-1-ol as impurity).

The unified aqueous phases were extracted with dicloromethane (3×20 mL) and the resulting organic phase was dried over Na₂SO₄ and concentrated in vacuum to yield the title compound (0.18 g, 40%) as a viscous oil (the sample contained ˜7% of 2-(4-aminocyclohexyl)ethan-1-ol as impurity).

¹H NMR (500 MHz, CDCl₃) δ_{H}: 4.77 (1H, m, OCHO), 4.10 (2H, m, 2×OCH), 3.82 (2H, m, 2×OCH), 2.98 and 2.63 (1H, br, CHN), 2.42 (2H, br, NH₂), 1.91-1.80 (2H, m), 1.72-1.41 (7H, m), 1.40-1.31 (2H, m), 1.20-1.07 ((1H, m), 1.05-0.95 (1H, m).

¹³C NMR (125 MHz, CDCl₃) δ_{C}: 101.21 and 101.01 (OCHO), 66.89 (2×OCH₂), 50.61 (CHNH), 42.16 (CH₂), 34.11 (2×CH₂), 32.06 (2×CH₂), 31.71 (CH), 25.82 and 25.68 (OCH₂CH₂CH₂O).

### 4-Methylcyclohexan-1-aminium chloride (compounds T·HCl+C·HCl (G=H))

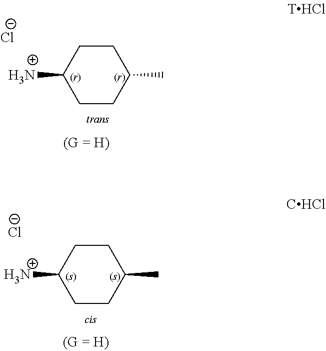

Reaction of 4-methylcyclohexane-1-one (K (G=H)) (5.5 ml, 5.00 g, 44.6 mmol) and 10% Pd/C (0.50 g) with ammonium formate (16.86 g, 267.5 mmol) in methanol (100 ml) afforded 4-methylcyclohexan-1-amine (compounds T+C (G=H)) (3.78 g, 75% yield) as colorless liquid. Lastly after the introducing of HCl-gas 4-methylcyclohexan-1-aminium chloride (compounds T·HCl+C·HCl (G=H)) (2.83 g, 43% yield, cis:trans=1.00:1.23 (¹H-NMR)) was formed as white solid.

### (1s,4s)-4-Methylcyclohexan-1-aminium chloride (cis-compound C·HCl (G=H))

¹H NMR (500 MHz, DMSO-d₆) δ_{H}: 8.12 (3H, br, NH₃⁺), 3.11 (1H, tt, J=6.8 Hz, J=3.9 Hz, CHₐₓ—NH₃⁺), 1.69-1.65 (2H, m, 2×CH), 1.64-1.61 (2H, m, 2×CH), 1.61-1.59 (H, m, CH—CH₃), 1.49-1.44 (2H, m, 2×CH), 1.42-1.35 (2H, m, 2×CH—CH₃), 0.90 (3H, t, J=6.8 Hz, CH₃);

¹³C NMR (125 MHz, DMSO-d₆) δ_{C}: 47.4 (CH—NH₃⁺), 28.2 (CH—CH₃), 28.1 (CH₂—CHCH₃), 26.1 (CH₂), 19.5 (CH₃);

### (1r,4r)-4-Methylcyclohexan-1-aminium chloride (trans-compound T·HCl (G=H))

¹H NMR (500 MHz, DMSO-d₆) δ_{H}: 8.12 (3H, br, NH₃⁺), 2.86 (1H, tt, J=11.8 Hz, J=4.0 Hz, CHₐₓ—NH₃⁺), 1.93-1.91 (2H, m, 2×CH_{eq}), 1.69-1.65 (2H, m, 2×CH_{eq}), 1.32 (2H, qd, J=12.7 Hz, J=3.3 Hz, 2×CHₐₓ), 1.26-1.22 (1H, m, CHₐₓ—CH₃), 0.94 (2H, qd, J=13.1 Hz, J=3.1 Hz, 2×CHₐₓ), 0.85 (3H, t, J=6.5 Hz, CH₃);

¹³C NMR (125 MHz, DMSO-d₆) δ_{C}: 49.1 (CH—NH₃⁺), 32.3 (CH₂—CHCH₃), 30.9 (CH—CH₃), 30.1 (CH₂), 21.8 (CH₃);

IR (liquid film) νₘₐₓ:2927, 2563, 2049, 161, 1512, 1455, 1392, 1127, 1029 cm⁻¹.

HRMS: M+H=114.12744 (delta=−2.5 ppm; C₇H16N). HR-ESI-MS-MS (CID=35%; rel. int. %): 97(100).

### 4-Ethylcyclohexan-1-aminium chloride (compounds T·HCl+C·HCl (G=Me))

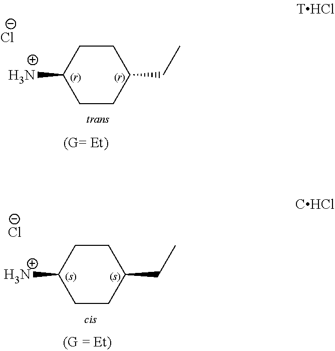

Reaction of 4-ethylcyclohexane-1-one (K (G=Me)) (5.6 ml, 5.00 g, 39.6 mmol) and 10% Pd/C (0.500 g) with ammonium formate (15.00 g, 237.7 mmol) in methanol (100 ml) afforded 4-ethylcyclohexan-1-amine (compounds T+C (G=Et)) (4.26 g, 85% yield, cis:trans=1.93:1.00 (¹H-NMR)) as colorless liquid. Lastly after the introducing of HCl-gas 4-ethylcyclohexan-1-aminium chloride (compounds T·HCl+C·HCl (G=Me)) was formed as white solid.

### (1s,4s)-4-Ethylcyclohexan-1-aminium chloride (cis-compound C·HCl (G=Me))

¹H NMR (500 MHz, DMSO-d₆) δ_{H}: 8.15 (3H, br, NH₃⁺), 3.12 (1H, br m CH_{eq}—NH₃⁺), 1.68-1.65 (2H, m, 2×CH), 1.64-1.60 (2H, m, 2×CH), 1.49-1.45 (2H, m, 2×CH), 1.44-1.41 (2H, m, 2×CH), 1.29 (1H, m, CHₐₓ—CH₂CH₃), 1.27 (2H, quint, J=7.2 Hz, CH₂CH₃), 0.85 (3H, t, J=7.3 Hz, CH₂CH₃);

¹³C NMR (125 MHz, DMSO-d₆)⁶c:47.6 (CH—NH₃⁺), 35.3 (CH—CH₂CH₃), 26.2 (CH₂), 25.9 (CH₂), 25.8 (CH₂CH₃), 11.3 (CH₂CH₃).

### (1r,4r)-4-Ethylcyclohexan-1-aminium chloride (trans-compound T·HCl (G=Me))

¹H NMR (500 MHz, DMSO-d₆) δ_{H}: 7.99 (3H, br, NH₃⁺), 2.87 (1H, br m CHₐₓ—NH₃⁺), 1.96-1.95 (2H, m, 2×CH_{eq}), 1.75-1.74 (2H, m, 2×CH_{eq}), 1.31 (2H, qd, J=12.8 Hz, J=3.4 Hz, 2×CHₐₓ), 1.18 (2H, quint, J=15 Hz CH₂CH₃), 1.07-1.03 (1H, m, CHₐₓ—CH₂CH₃), 0.91 (2H, qd, J=12.9 Hz, J=3.3 Hz, 2×CHₐₓ), 0.85 (3H, t, J=7.5 Hz, CH₂CH₃);

¹³C NMR (125 MHz, DMSO-d₆) δ_{C}: 49.4 (CH—NH₃⁺), 37.5 (CH—CH₂CH₃), 30.1 (CH₂), 29.9 (CH₂—CHCH₂CH₃), 28.7 (CH₂CH₃), 11.6 (CH₂CH₃);

IR (liquid film) νₘₐₓ: 2933, 2575, 2047, 1583, 1505, 1453, 1388, 1236, 1121, 1036 cm⁻¹.

HRMS: M+H=128.14303 (delta=−2.7 ppm; C₈H₁₈N). HR-ESI-MS-MS (CID=35%; rel. int. %): 111(100) and 69(9).

### 4-Phenylcyclohexan-1-aminium chloride (compounds T·HCl+C·HCl (G=Ph))

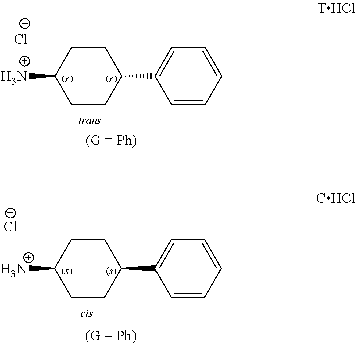 [SMILES: [Cl-] ; [NH3+][C@H]1CC[C@H](c2ccccc2)CC1 ; [Cl-] ; [NH3+][C@H]1CC[C@@H](c2ccccc2)CC1]

Reaction of 4-phenylcyclohexane-1-one one (K (G=Ph)) (4.00 g, 22.9 mmol) and 10% Pd/C (0.40 g) with ammonium formate (8.66 g, 137.4 mmol) in methanol (80 ml) afforded 4-phenylcyclohexan-1-amine (compounds T+C (G=Ph)) (2.93 g, 73% yield, cis/trans=1.00:3.70) as liquid. Lastly after the introducing of HCl 4-phenylcyclohexan-1-aminium chloride (compounds T·HCl+C·HCl (G=Ph)) (2.2 g, 45% yield) was formed as white solid.

### (1s,4s)-4-Phenylcyclohexan-1-aminium chloride (cis-compound C·HCl (G=Ph))

¹H NMR (500 MHz, DMSO-d₆) δ_{H}: 8.03 (3H, br, NH₃⁺), 7.34-7.32 (H, m, ArHₒᵣₜₒ), 7.31-7.27 (H, m, ArHₘₑₜₐ), 7.19-7.17 (H, m, ArHₚₐᵣₐ), 3.42-3.41 (1H, m, CH_{eq}—NH₃⁺), 2.57 (1H, tt, J=11.4 Hz, J=3.4 Hz, CHₐₓ-Ph),

¹³C NMR (126 MHz, DMSO-d₆) δ_{C}: 146.2 (ArC), 128.2 (ArCHₘₑₜₐ), 126.9 (ArCHₒᵣₜₒ), 125.9 (ArCHₚₐᵣₐ), 45.8 (CH—NH₃⁺), 41.7 (CH-Ph), 27.8 (CH₂); 26.6 (CH₂);

### (1r,4r)-4-Phenylcyclohexan-1-aminium chloride (trans-compound T·HCl (G=Ph))

¹H NMR (500 MHz, DMSO-d₆) δ_{H}: 8.03 (3H, br, NH₃⁺), 7.31-7.27 (H, m, ArHₘₑₜₐ), 7.24-7.23 (H, m, ArHₒᵣₜₒ), 7.19-7.17 (H, m, ArHₚₐᵣₐ), 3.06 (1H, tt, J=11.6 Hz, J=3.9 Hz, CHₐₓ—NH₃), 2.47 (1H, tt, J=12.0 Hz, J=3.4 Hz, CHₐₓ-Ph),

¹³C NMR (126 MHz, DMSO-d₆) δ_{C}: 146.0 (ArC), 128.2 (ArCHₘₑₜₐ), 126.6 (ArCHₒᵣₜₒ), 126.0 (ArCHₚₐᵣₐ), 48.9 (CH—NH₃⁺), 42.2 (CH-Ph), 31.4 (CH₂); 30.4 (CH₂);

HRMS: M+H=176.14302 (delta=−2.0 ppm; C₁₂H18N). HR-ESI-MS-MS (CID=35%; rel. int. %): 159(100); 91(3) and 81(3);

IR (liquid film) νₘₐₓ: 2939, 2544, 2038, 1610, 1504, 1451, 1390, 1182, 1073, 1020, 758, 700 cm⁻¹.

### 4-Benzylcyclohexan-1-aminium chloride (compounds T·HCl+C·HCl (G=CH₂Ph))

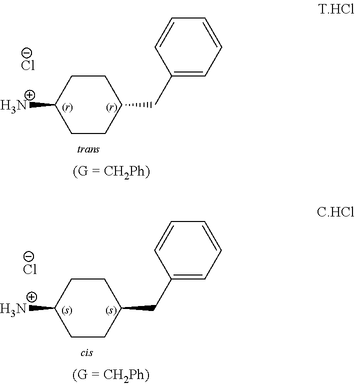 [SMILES: [NH3+][C@H]1CC[C@H](Cc2ccccc2)CC1 ; [Cl-] ; [NH3+][C@H]1CC[C@@H](Cc2ccccc2)CC1 ; [Cl-]]

Reaction of 4-benzylcyclohexyl-1-one (K (G=CH₂Ph)) (1.50 g, 7.97 mmol) and 10% Pd/C (0.45 g) with ammonium formate (3.01 g, 47.8 mmol) in methanol (60 ml) afforded 4-benzylcyclohexan-1-amine (compounds T+C (G=CH₂Ph)) (0.29 g, 19% yield, cis/trans=1.00:1.08 (¹H-NMR)) as liquid. Lastly after the introducing of HCl-gas 4-benzylcyclohexan-1-aminium chloride (compounds T·HCl+C·HCl (G=CH₂Ph)) (0.22 g, 12% yield) was formed as white solid.

### (1s,4s)-4-Benzylcyclohexan-1-aminium chloride (cis-compound C·HCl (G=CH₂Ph))

¹H NMR (500 MHz, DMSO-d₆) δ_{H}: 8.14 (3H, br, NH₃⁺), 7.29-7.25 (2H, m, ArHₘₑₜₐ), 7.19-7.13 (3H, m, ArHₚₐᵣₐ, ArHₒᵣₜₒ), 3.14-3.13 (1H, m, CH—NH₃⁺), 2.55 (2H, d, J=7.64 Hz, CH₂-Ph), 1.78-1.71 (3H, m, 2 CH, CHₐₓ—CH₂Ph), 1.66-1.59 (2H, m, 2×CH), 1.44-1.41 (4H, m, 4×CH);

¹³C NMR (125 MHz, DMSO-d₆) δ_{C}: 140.6 (ArC), 128.7 (ArCₒᵣₜₒ), 128.1 (ArCₘₑₜₐ), 125.7 (ArC, para), 47.6 (CH—NH₃⁺), 39.3 (CH₂-Ph), 35.4 (CH—CH₂-Ph), 26.0 (CH₂), 25.9 (CH₂);

### (1r,4r)-4-Benzylcyclohexan-1-aminium chloride (trans-compound T·HCl (G=CH₂Ph))

¹H NMR (500 MHz, DMSO-d₆) δ_{H}: 8.14 (3H, br, NH₃⁺), 7.29-7.25 (2H, m, ArHₘₑₜₐ), 7.19-7.13 (3H, m, ArHₒᵣₜₒ, ArHₚₐᵣₐ), 2.88 (1H, tt, J=11.8 Hz, J=3.2 Hz, CHₐₓ—NH₃⁺), 2.45 (2H, d, J=6.9 Hz, CH₂-Ph), 1.93-1.91 (2H, m, 2×CH_{eq}), 1.66-1.59 (2H, m, 2×CH_{eq}), 1.44-1.41 (1H, m, CHₐₓ—CH₂Ph), 1.28 (2H, qd, J=12.61 Hz, J=3.2 Hz, 2×CHₐₓ), 1.00 (2H, qd, J=13.58 Hz, J=2.9 Hz, 2 CHₐₓ);

¹³C NMR (125 MHz, DMSO-d₆) δ_{C}: 140.3 (ArC), 128.8 (ArCₒᵣₜₒ), 128.0 (ArCₘₑₜₐ), 125.7 (ArCₚₐᵣₐ), 49.3 (CH—NH₃⁺), 42.3 (CH₂-Ph), 37.9 (CH—CH₂Ph), 30.0 (CH₂), 29.9 (CH₂),

HRMS: M+H=190.15850 (delta=−2.8 ppm; CO₃H₂ON). HR-ESI-MS-MS (CID=35%; rel. int. %): 173(100); 117(2); 105(31); 95(9); 91(5) and 81(2).

IR (liquid film) νₘₐₓ: 3073, 2610, 2035, 1610, 1511, 1494, 1453, 1392, 1347, 1203, 1062, 744, 701 cm⁻¹.

### cis-Diastereomer of 2-(4-aminocyclohexyl)acetic acid ethyl ester hydrochloride (compound IIb·HCl)

### (1s,4s)-4-(2-Ethoxy-2-oxoethyl)cyclohexan-1-aminium chloride (compound IIb·HCl)

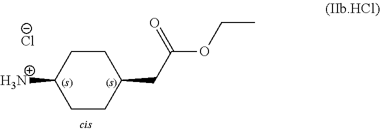

The cis-diastereomer of 2-(4-aminocyclohexyl)acetic acid ethyl ester hydrochloride (IIb·HCl with de ˜90.2%) was obtained from the mother liquor of the recrystallization of the diatereomeric mixture of 2-(4-aminocyclohexyl)acetic acid ethyl ester hydrochloride (Ib·HCl IIb·HCl in ˜1:1 ratio) at industrial scale production process according to WO2010/070368.

### (1s,4s)-4-(2-Ethoxy-2-oxoethyl)cyclohexan-1-aminium chloride (cis-compound IIb·HCl)

¹H NMR (500 MHz, DMSO-d₆) δ_{H}: 8.14 (3H, br, NH₃⁺), 4.09-4.02 (2H, m, OCH₂), 3.18-3.09 (1H, m, CHₐₓ—NH₃⁺), 2.27 (2H, d, J=7.5 Hz, CH₂COOEt), 1.98-1.86 (1H, m, CH_{eq}—CH₂COOEt), 1.69-1.62 (4H, m, 4×CH), 1.53-1.43 (4H, m, 4×CH), 1.18 (3H, t, J=7.2 Hz, CH₃).

¹³C NMR (126 MHz, DMSO-d₆) δ_{C}: 171.96 (CO), 59.62 (OCH₂), 47.2 (CH—NH₃⁺), 37.92 (CH₂COOEt), 30.58 (CHₐₓ—CH₂COOEt), 25.96 (CH₂), 25.89 (CH₂), 14.04 (CH₃).

IR (neat) νₘₐₓ: 2927, 1727, 1601, 1520, 1511, 1447, 1375, 1226, 1165, 1031 cm¹.

### Transaminases of Different Microbial Strains as Biocatalysts

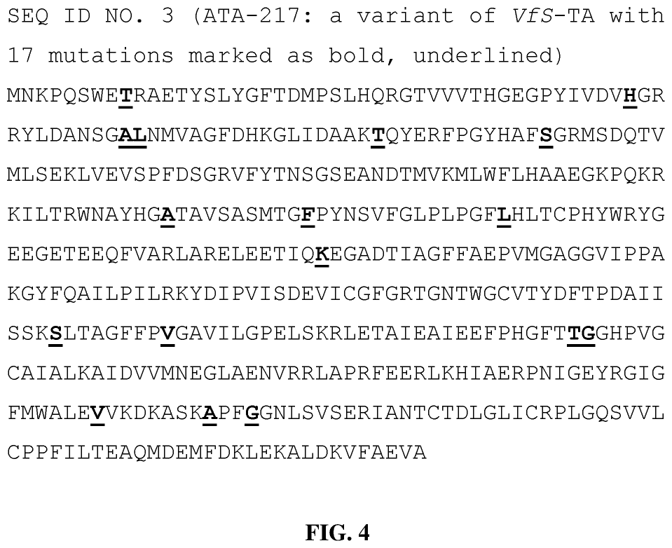

Generation of plasmids and expression of an (S)-selective transaminase from Chromobacterium violaceum (CvS-TA) was disclosed by K. E. Cassimje et al. (ACS Catal. 1(9), 1051-1055 (2011), DOI: 10.1021/cs200315h). Recombinant expression of His-tagged CvS-TA W60C mutant (CvS_{W60C}-TA) exhibiting enhanced catalytic properties was published by K. E. Cassimje et al. (Org. Biomol. Chem., 10, 5466-5470 (2012), DOI: 10.1039/C2OB25893E). Recombinant expression of His-tagged VfS-TA was unveiled by F. G. Mutti et al. (Eur. J. Org. Chem., 1003-1007 (2012), DOI: 10.1002/ejoc.201101476). Production and whole cell immobilization of three (R)- and three (S)-selective TAs, the (R)-selective TAs from Arthrobacter sp. (ArR-TA), its mutated variant (ArRₘᵤₜ-TA), Aspergillus terreus (AtR-TA); and the (S)-selective TAs from Arthrobacter citreus (ArS-TA), a mutated variant of Chromobacterium violaceum (CvS_{W60C}-TA), Vibrio fluvialis (VfS-TA), respectively, applied for kinetic resolution of racemic amines in immobilized whole-cell form was published by Z. Molnár et al. (Catalysts, 9, 438 (2019), DOI: 10.3390/cata19050438). A Transaminase Screening Kit (Codexis, Redwood City, USA) containing 24 mutant amine transaminases (ATAs) from two different parent lineages: Vibrio fluvialis JS17 ATA (VfS-TA: Biotechnol. Bioeng. 65, 206-211 (1999), DOI: 10.1002/(SICI)1097-0290(19991020)65:2<206::AID-BIT11>3.0.CO; 2-9) and Arthrobacter sp. ATA (ArR-TA: Appl. Microbiol. Biotechnol. 69, 499-505 (2006), DOI: 10.1007/s00253-005-0002-1) was also assayed. The 17 mutations (marked as bold in SEQ ID NO. 3; see FIG. 4) in a variant of VfS-TA, named as ATA-217 were disclosed by Novick S. J. et al (ACS Catal. 11, 3762-3770 (2021), DOI: 10.1021/acscatal.0c05450).

### Expression of Transaminases

Production of ArS-TA and VfS-TA was achieved in E. coli BL21(DE3) containing the recombinant pASK-IBA35+plasmid with the gene of the given TA. LB-Car medium (5 mL; LB medium containing carbenicillin, 50 mg L⁻¹) was inoculated with one fresh colony from an overnight LB-Car agar plate and cells were grown overnight in shake flask (37° C., at 200 rpm). LB medium (0.5 L) in a 2 L flask was inoculated with seed culture (2 mL) and cells were grown at 37° C., 200 rpm until the OD₆₄₀ reached 0.8 (approx. 4 h). For induction, tetracycline solution (20 μL, 5 mg ml⁻¹ tetracycline in ethanol) was added and the culture was shaken for further 16 h at 25° C., 200 rpm. The cells were then harvested by centrifugation (15,000 g, 4° C., 20 min).

Production of AtR-TA, ArR-TA, ArRmut-TA and CvS_{W60C}-TA was achieved in E. coli BL21(DE3) containing the recombinant pET21a plasmid with the gene of the given TA. LB-Car medium (5 mL; LB medium containing carbenicillin, 50 mg L⁻¹) was inoculated with one fresh colony from an overnight LB-Car agar plate and cells were grown overnight in shake flask (37° C., at 200 rpm). Autoinduction medium (0.5 L: Na₂HPO₄, 6 g L⁻¹; KH₂PO₄, 3 g L⁻¹; tryptone, 20 g L⁻¹; yeast extract, 5 g L-; NaCl, 5 g L⁻¹; glycerol, 7.56 g L⁻¹; glucose, 0.5 g L⁻¹; lactose, 2 g L^{-1 S}) in a 2 L flask was inoculated with seed culture (2 mL) and was shaken for 16 h at 25° C., 200 rpm. The cells were then harvested by centrifugation (15,000 g, 4° C., 20 min).

### Immobilization of Transaminase-Expressing Whole-Cells

The silica sol was prepared as follows: TEOS (14.4 mL) was added to a solution containing 0.1 M HNO₃ (1.3 mL) and distilled water (5 mL) and the resulted mixture was sonicated for 5 min at room temperature (Emag Emmi 20HC Ultrasonic Bath, 45 kHz) and kept at 4° C. for 24 h. Then MAT540 support (3 g) was mixed with a cell paste suspension (6 mL; taken from 1 g of centrifuged cell paste resuspended in 6 ml of 0.1 M phosphate buffer, pH 7.5), and the resulted suspension was shaken intensively until become homogeneous (Technokartell Test Tube Shaker Model T3SK, 40 Hz, room temperature, 5 min). Finally, the homogenized supported cell suspension was mixed with the silica sol and the resulted mixture was shaken intensively (Technokartell Test Tube Shaker Model T3SK, 40 Hz, room temperature, 5 min). Gelation occurred within 30 min at room temperature, followed by aging the gel at 4° C. for 48 h in an open dish. The crude immobilized TA biocatalyst was washed with distilled water (2×15 mL, 100 mM, pH 7.5), dried at room temperature (24 h), and stored at 4° C.

Purification of the W60C Mutant of Transaminase from Chromobacterium violaceum (CvS_{W60C}-TA)

After fermentation of E. coli cells containing CvS_{W60C}-TA, cells were disrupted by French press, centrifuged and crude cell extract was purified by Ni-NTA resin as described previously by F. G. Mutti et al. (Eur. J. Org. Chem., 1003-1007 (2012), DOI: 10.1002/ejoc.201101476). Cofactor PLP was added to stock solutions of CvS_{W60C}-TA which were kept at −20° C. in 20% glycerol solution until further use.

Purification of the Transaminase from Vibrio fluvialis (VfS-TA)

After fermentation of E. coli cells containing VfS-TA, cells were disrupted by French press, centrifuged and crude cell extract was purified by Ni-NTA resin as described above for the purification of CvS_{W60C}-TA. Cofactor PLP was added to stock solutions of VfS-TA which were kept at −20° C. in 20% glycerol solution until further use.

Surface Activation of Aminoethyl Polymethacrylate Resins with Glycerol Diglycidyl Ether (GDE)

According to the method of E. Abahizi et al. (Biochem. Eng. J. 132, 270-278 (2018), DOI: 10.1016/j.bej.2018.01.022)), ethyleneamine-functionalized methacrylic polymer resins ReliZyme™ EA403/S (1.0 g, particle size 150-300 μm, pore size 400-600 A), were added to a glycerol diglycidyl ether solution (10 mmol) in ethanol (15 mL). The suspension of polymer support in bisepoxide solution was shaken at 450 rpm for 24 h at 25° C. The activated support was filtered off on a glass filter (G3), washed with Patosolv© (3×10 mL), dried at room temperature (4 h) and stored at 4° C. under argon atmosphere.

Immobilization of CvS_{W60C}-TA on GDE-activated aminoethyl resins

According to the method of E. Abahizi et al. (Biochem. Eng. J. 132, 270-278 (2018), DOI: 10.1016/j.bej.2018.01.022)), in an Eppendorf tube (1.5 mL) purified CvS_{W60C}-TA (210 μL, 4.8 mg mL⁻¹) was diluted with HEPES buffer (790 μL, 50 mM, pH 7.0), and then the GDE-activated aminoethyl resin (10.0 mg, resulting in enzyme:support ratio=1:10) was added to the solution. The resulted suspension was shaken at 900 rpm for 24 h at 25° C. The immobilized CvS_{W60C}-TA was centrifuged, washed with HEPES buffer (2×1.0 mL). Protein concentrations of the CvS_{W60C}-TA solution before immobilization and in the supernatant were determined by a NanoDrop 2000 spectrophotometer. Immobilization yield (IY) was calculated according to equation IY(%)=(P₀−P)/P₀×100 (where P₀ [mg mL⁻¹] is the initial protein concentration before immobilization, and P [mg mL⁻¹] is the protein concentration in supernatant after immobilization). Because after immobilization of the purified native CvS_{W60C}-TA onto the GDE-activated aminoethyl resin (EA-G) negligible protein concentration could be detected (P ˜0 mg mL⁻¹), immobilization yield at enzyme:support ratio=1:10 was ˜100%. After immobilization, the resulted covalently immobilized CvS_{W60C}-TA biocatalyst was used immediately in dynamic isomerization reactions.

The immobilization process could be upscaled tenfold in 4 mL vials with identical results.

### Immobilization of VfS-TA on GDE-Activated Aminoethyl Resins

According to the method of E. Abahazi et al. (Biochem. Eng. J. 132, 270-278 (2018), DOI: 10.1016/j.bej.2018.01.022)), in an Eppendorf tube (1.5 mL) purified VfS-TA (250 μL, 4.5 mg mL⁻¹) was diluted with HEPES buffer (790 μL, 50 mM, pH 7.0), and then the GDE-activated aminoethyl resin (10.0 mg, resulting in enzyme:support ratio=1:10) was added to the solution. The resulted suspension was shaken at 900 rpm for 24 h at 25° C. The VfS-TA was centrifuged, washed with HEPES buffer (2×1.0 mL). Protein concentrations of the VfS-TA solution before immobilization and in the supernatant were determined by a NanoDrop 2000 spectrophotometer. An immobilization yield of ˜100% at enzyme:support ratio=1:10 was observed. After immobilization, the resulted covalently immobilized VfS-TA biocatalyst was used immediately in dynamic isomerization reactions.

### Continuous-Flow Immobilization of CvS_{W60C}-TA on GDE-Activated Aminoethyl Resins

According to the method of E. Abahazi et al. (Biochem. Eng. J. 132, 270-278 (2018), DOI: 10.1016/j.bej.2018.01.022)), flow-through immobilization of CvS_{W60C}-TA was performed in a laboratory scale flow reactor built from a Knauer Azura P4.1S isocratic HPLC pump attached to CatCart™ columns filled with the EA-G supports in an in-house made aluminum metal block column holder with precise temperature control. CvS_{W60C}-TA solution (2 mg mL⁻¹, in a volume corresponding to enzyme:support ratio 1:10) was recirculated in stainless-steel CatCart™ columns filled with EA-G support (stainless steel, inner diameter: 4 mm; total length: 70 mm; packed length: 65 mm; inner volume: 0.816 mL; support weights: 211.4±16.1 mg) at a flow rate of 0.5 mL min⁻¹. Protein concentrations of the CvS_{W60C}-TA solution before immobilization and at several time points during immobilization were determined by a Nano-Drop 2000 spectrophotometer.

Dynamic Isomerization (DI) of Trans/Cis-Diastereomeric Mixture of 2-(4-Aminocyclohexyl)-Acetic Acid Ethyl Esters (Compounds Ib+IIb) with a Transaminase in Batch Mode

### Example 1

DI of Trans/Cis-Ethyl Esters (Ib+IIb) with Immobilized Whole Cell CvS_{W60C}-TA in Presence of Pyruvate in Batch Mode

The immobilized whole cell Chromobacterium violaceum transaminase W60C mutant biocatalyst (CvS_{W60C}-TA, 50 mg) was suspended in phosphate buffer (1.6 mL, 100 mM, pH 7.5) in a 4 ml vials. The cis/trans diastereomeric mixture of 2-(4-aminocyclohexyl)acetic acid ethyl ester hydrochloride salt [compounds IIb·HCl+Ib·HCl, in 44:56 ratio; 11.1 mg, 50 μmol, in phosphate buffer (200 μL, 100 mM, pH 7.5)] and sodium pyruvate as amine acceptor [0.5 eq., 0.23 mg, 25 μmol, in phosphate buffer (200 μL, 100 mM, pH 7.5)] were added to the biocatalyst suspension providing a final reaction volume of 2 mL with 25 mM of cis/trans diastereomeric mixture (IIb·HCl/Ib HC). The reaction mixture was shaken on an orbital shaker (500 rpm) at 30° C. for 24 h. To the samples taken from the reaction mixture (150 μL), sodium hydroxide (100 μL, 1 M) was added, followed by extraction with ethyl acetate (800 μL). Derivatization of the amines was performed by the addition of acetic anhydride (20 μL, 60° C., 1 h), then the organic phase was dried over Na₂SO₄. Samples were analyzed by gas chromatography.

According to integration of peak areas for the ketone (IIIb) and the corresponding acetamides of Ib and IIb, the molar fractions of the products Ib, IIb, and IIIb were in the mixture 76.3%, 0.6% and 23.0%, respectively.

The reaction mixture was centrifuged to remove the biocatalyst. The aqueous supernatant was acidified by addition of aqueous cc. HCl to pH 1, and it was extracted with dichloromethane (3×3 mL). The unified organic phases were washed with saturated brine (3 mL) and dried over anhydrous Na₂SO₄ and concentrated in vacuum to yield the ketone (compound IIIb: 2.0 mg, 11 μmol, 95% yield). The pH of the acidified aqueous phase was adjusted pH 10 by addition of 25% aqueous ammonium hydroxide and the basic solution was extracted with dichloromethane (3×3 mL). The unified organic phase was washed with saturated brine (3 mL) and dried over anhydrous Na₂SO₄ and concentrated in vacuum to yield the trans-amine (compound Ib: 4.8 mg, 26 μmol, 68% yield with deₜᵣₐₙₛ=98.3% by GC).

### Example 2

DI of Trans/Cis-Ethyl Esters (Ib+IIb) with Immobilized Whole Cell VfS-TA in Presence of Pyruvate in Batch Mode

The procedure was performed as presented in Example 1 modified in a way that immobilized whole cell Vibrio fluvialis transaminase (VfS-TA, 50 mg) biocatalyst was used.

After 24 h reaction time, according to integration of peak areas for the ketone (IIIb) and the corresponding acetamides of Ib and IIb, the molar fractions of the products Ib, IIb, and IIIb were in the mixture 70.6%, 1.5%, 27.9% and respectively.

### Example 3

DI of Trans/Cis-Ethyl Esters (Ib+IIb) with Doubled Amount of Immobilized Whole Cell VfS-TA in Presence of Pyruvate in Batch Mode

The procedure was performed as presented in Example 1 modified in a way that immobilized whole cell Vibrio fluvialis transaminase (VfS-TA, 100 mg) biocatalyst was used.

After 6 h reaction time, according to integration of peak areas for the ketone (IIIb) and the corresponding acetamides of Ib and IIb, the molar fractions of the products Ib, 1Ib, and IIIb were in the mixture 74.5%, 1.0% and 24.5%, respectively.

### Example 4

DI of Trans/Cis-Ethyl Esters (Ib+IIb) with Purified Soluble CvS_{W60C}-TA in Presence of Pyruvate in Batch Mode

The procedure was performed as presented in Example 1 modified in a way that Ni-NTA-purified Chromobacterium violaceum transaminase W60C mutant (CvS_{W60C}-TA) biocatalyst in solution was used (at 0.5 mg/ml protein concentration in the final reaction mixture, supplemented with 0.2 mM piridoxal-5-phosphate (PLP)) in the reaction starting from 25 mM of cis/trans diastereomeric mixture (IIb·HCl/Ib·HCl=44:56).

After 2 h reaction time, according to integration of peak areas for the ketone (IIIb) and the corresponding acetamides of Ib and IIb, the molar fractions of the products Ib, IIb, and IIIb were in the mixture 86.0%, 0% and 14.0%, respectively.

Extractive workup as presented in Example 1 gave ketone (compound IIIb: 1.3 mg, 7 μmol, ˜98% yield) trans-amine (compound Ib: 4.9 mg, 27 μmol, 62% yield with deₜᵣₐₙₛ>99% by GC).

### Example 5

DI of Trans/Cis-Ethyl Esters (Ib+IIb) with Purified Soluble VfS-TA in Presence of Pyruvate in Batch Mode

The procedure was performed as presented in Example 1 modified in a way that Ni-NTA-purified Vibrio fluvialis transaminase (VfS-TA) biocatalyst was used (at 0.5 mg/ml protein concentration in the final reaction mixture, supplemented with 0.2 mM piridoxal-5-phosphate (PLP)) in the reaction starting from 25 mM of cis/trans diastereomeric mixture (IIb·HCl/Ib·HCl=51:49).

After 3 h reaction time, according to integration of peak areas for the ketone (IIIb) and the corresponding acetamides of Ib and IIb, the molar fractions of the products Ib, IIb, and IIIb in the mixture were 79.0%, 0.5% and 20.5%, respectively.

Extractive workup as presented in Example 1 gave ketone (compound IIIb: 1.7 mg, ˜9 μmol, ˜92% yield) trans-amine (compound Ib: 4.8 mg, 26 μmol, 65% yield with deₜᵣₐₙₛ=98.7% by GC).

### Example 6

DI of Trans/Cis-Ethyl Esters (Ib+IIb) with VfS-TA Covalently Immobilized on Porous Resin in Presence of Pyruvate in Batch Mode

The procedure was performed as presented in Example 1 modified in a way that covalently immobilized Vibrio fluvialis transaminase on polymer resin (VfS-TA, 10 mg) as biocatalyst was used.

After 6 h reaction time, according to integration of peak areas for the ketone (IIIb) and the corresponding acetamides of Ib and IIb, the molar fractions of the products Ib, 1Ib, and IIIb in the mixture were 74.9%, 1.6% and 23.6%, respectively.

### Example 7

DI of Trans/Cis-Ethyl Esters (Ib+IIb) with Immobilized Whole Cell VfS-TA in Presence of Ketone (IIIb) in Batch Mode

The procedure was performed as presented in Example 1 modified in a way that immobilized whole cell Vibrio fluvialis transaminase (VfS-TA, 50 mg) as biocatalyst and ethyl 2-(4-oxocyclohexyl)acetate (compound IIIb, 2.5 mM) as the amine acceptor were used in the reaction.

After 48 h reaction time, according to integration of peak areas for the ketone (IIIb) and the corresponding acetamides of Ib and IIb, the molar fractions of the products Ib, 1Ib, and IIIb were in the mixture 74.6%, 12.4% and 13.0%, respectively.

### Example 8

DI of Trans/Cis-Ethyl Esters (Ib+IIb) with Purified Soluble VfS-TA in Presence of Ketone (IIIb) in Batch Mode

The procedure was performed as presented in Example 1 modified in a way that ethyl 2-(4-oxocyclohexyl)acetate (compound IIIb, 2.5 mM) as the amine acceptor and Ni-NTA-purified Vibrio fluvialis transaminase (VfS-TA) biocatalyst (at 0.5 mg/ml protein concentration in the final reaction mixture, supplemented with 0.2 mM piridoxal-5-phosphate (PLP)) were used in the reaction starting from 25 mM of cis/trans diastereomeric mixture (IIb·HCl/Ib·HCl=51:49).

After 48 h reaction time, according to integration of peak areas for the ketone (IIIb) and the corresponding acetamides of Ib and IIb, the molar fractions of the products Ib, 1Ib, and 11Ib were in the mixture 78.4%, 10.8% and 10.7%, respectively.

Extractive workup as presented in Example 1 gave ketone (compound IIIb: 1.0 mg, ˜5.4 μmol, ˜97% yield) and crude trans-amine (compound Ib with minor amount of IIb): 6.2 mg, 33 gmol, 66% yield with deₜᵣₐₙₛ=75.7% by GC).

Recrystallization of the crude trans-amine (compound Ib with minor amount of IIb) according to the method disclosed in WO2010/070368 gave trans-amine hydrochloride salt (compound Ib·HCl: 5.7 mg, 26 μmol, with deₜᵣₐₙₛ>99% by GC).

### Example 9

DI of Trans/Cis-Ethyl Esters (Ib+IIb) with Purified Soluble VfS-TA in Presence of Cyclohexanone in Batch Mode

The procedure was performed as presented in Example 1 modified in a way that cyclohexanone (5 mM) as the amine acceptor and Ni-NTA-purified Vibrio fluvialis transaminase (VfS-TA) biocatalyst (at 0.5 mg/ml protein concentration in the final reaction mixture, supplemented with 0.2 mM piridoxal-5-phosphate (PLP)) were used in the reaction starting from 25 mM of cis/trans diastereomeric mixture (IIb·HCl/Ib·HCl=51:49).

After 48 h reaction time, according to integration of peak areas for the ketone (IIIb) and the corresponding acetamides of Ib and IIb, the molar fractions of the products Ib, IIb, and IIIb were in the mixture 85.9%, 9.3% and 4.8%, respectively.

Extractive workup as presented in Example 1 gave crude ketone (compound IIIb, with minor amount of cyclohexanone: 0.4 mg) and crude trans-amine (compound Ib, with minor amount of IIb): 6.1 mg, 32 μmol, 64% yield with deₜᵣₐₙₛ=80.4% by GC).

Dynamic Isomerization of Trans/Cis-Diastereomeric Mixture of 4-Substituted Cyclohexane-1-Amines (Compounds C+T) with Covalently Immobilized W60C Mutant of Transaminase from Chromobacter violaceum in Continuous Flow Mode

### Example 10

DI of Trans/Cis-Ethyl Esters (Ib·HCl+IIb·HCl) with CvS_{W60C}-TA Covalently Immobilized on Porous Resin in Presence of Pyruvate in Continuous Flow Mode

The dynamic isomerization of the cis/trans-diastereomeric mixture of 4-(2-ethoxy-2-oxoethyl)cyclohexan-1-aminium chloride (compounds Ib·HCl+IIb·HCl) was accomplished in a laboratory scale flow reactor comprised of syringe pump (Asia© Syringe Pump system, Syrris Ltd., Royston, UK) attached to SynBioCart columns (SynBiocat, Budapest, Hungary; stainless steel outer and PTFE inner tube, inner diameter: 4 mm; total length: 70 mm; packed length: 65 mm; inner volume: 0.816 mL). The column was sealed by filter membranes made of PTFE [Whatman® Sigma-Aldrich, WHA10411311, pore size 0.45 μm]. The sealing elements were made of PTFE. PTFE tubing ( 1/16″ outer diameter and 0.8 mm inner diameter, VICI AG International, Schenkon, Switzerland) and PEEK fingertight (Sigma Aldrich) were used to connect columns (purchased from commercial vendors). Three serially connected SynBioCart columns filled with the covalently immobilized CvS_{W60C}-TA biocatalyst (filling weights: 375±12 mg/column) immobilized on glycerol-1,3-diglycidyl ether modified methacrylic polymer resins (ReliZyme™ EA403/S; polymethyl methacrylate supports, particle size 150-300 μm, pore size 400-600 A) were thermostated at 40° C. with precise temperature control in an in-house made stainless steel metal block. The CvS_{W60C}-TA biocatalyst-filled columns were prewashed by HEPES buffer (50 mM, pH=7.0) for 1 h. Then the solution of the cis/trans-diastereomeric mixture of 4-(2-ethoxy-2-oxoethyl)cyclohexan-1-aminium chloride [compounds IbHC+IIb·HCl, cis:trans=69.7:30.3, 20 mM, dissolved in HEPES buffer (50 mM, pH=7.0) containing DMSO as cosolvent (10% v/v), sodium pyruvate (0.95 eq.) and PLP (1% n/n)] was pumped through the column at a flow rate of 10 μL min⁻¹. After the stationary operation was established (˜6 h), samples were taken and analyzed by GC at every hour during the stationary operation period, and the outflowing reaction products were collected for 48 h.

The collected solution (25 mL) was acidified by aqueous cc. HCl to pH 1, and the formed ketone (compound IIIb) was removed by extraction with dichloromethane (3×50 mL). After removal of the ketone, the aqueous phase was basified by addition of ammonium hydroxide (25%) to pH 12 and the residual amine was extracted with dichloromethane (3×50 mL). The unified organic phase was extracted with saturated brine (30 mL) and dried over Na₂SO₄ and concentrated in vacuum to yield the product amine (compound Ib) which was dissolved in diethyl ether and treated with HCl-gas. The precipitate was then isolated by filtration and dried to give the trans-amine hydrochloride salt product (compound Ib·HCl, 11.6 mg, isolated yield 27%, deₜᵣₐₙₛ>99%) as a white solid.

¹H NMR (500 MHz, DMSO-d₆) δ_{H}: 8.09 (3H, br, NH₃′), 4.04 (2H, q, J=7.22 Hz, OCH₂), 2.94-2.83 (1H, m, CHₐₓ—NH₃⁺), 2.17 (2H, d, J=7.0 Hz, CH₂—COOEt), 1.93 (2H, br d, J=13.5 Hz, 2×CH_{eq}CHNH₃⁺), 1.72 (2H, br d, J=13.0 Hz, 2×CH_{eq}), 1.64-1.56 (1H, m, CHₐₓ—CH₂COOEt), 1.32 (2H, qd, J=12.4 Hz, J=2.9 Hz, 2×CHₐₓ—CHNH₃⁺), 1.17 (3H, t, J=7.2 Hz, CH₃), 1.02 (2H, qd, J=12.8 Hz, J=2.7 Hz, 2×CHₐₓ);

¹³C NMR (125 MHz, DMSO-d₆) δ_{C}: 171.8 (CO), 59.6 (OCH₂), 48.9 (CH—NH₃⁺), 40.4 (CH₂—COOEt), 33.1 (CH₂), 29.8 (2×CH₂), 14.0 (CH₃);

HRMS: M+=200.16443 (delta=−0.4 ppm; C₁₁H₂₂O₂N). HR-ESI-MS-MS (CID=35%; rel. int. %): 183(41) and 141(100).

### Example 11

DI of Trans/Cis-Isopropyl Esters (Id·HCl+IId·HCl) with CvS_{W60C}-TA Covalently Immobilized on Porous Resin in Presence of Pyruvate in Continuous Flow Mode

The procedure was performed as presented in Example 10 modified in a way that that four serially connected SynBioCart columns filled with the covalently immobilized CvS_{W60C}-TA biocatalyst were applied for the dynamic isomerization of the cis/trans-diastereomeric mixture of 4-(2-isopropoxy-2-oxoethyl)cyclohexan-1-aminium chloride (compounds Id·HCl+IId·HCl, 20 mM, cis:trans=51.7:48.3). The stationery operation of the reaction for 48 h afforded the trans-amine hydrochloride salt product (compound Id·HCl, 18.7 mg, isolated yield 30%, deₜᵣₐₙₛ>99%) as a white solid.

¹H NMR (500 MHz, DMSO-d₆) δ_{H}: 8.05 (3H, br, NH₃⁺), 4.88 (1H, quint, J=6.3 Hz, CH—(CH₃)₂), 2.89-2.87 (1H, m, CHₐₓ—NH₃⁺), 2.14 (2H, d, J=6.96 Hz, CH₂—COOⁱPr), 1.94-1.91 (2H, m, 2×CH_{eq}), 1.72-1.70 (2H, m, 2×CH_{eq}), 1.63-1.55 (1H, m, CHₐₓ—CH₂COOⁱPr), 1.32 (2H, qd, J=12.7 Hz, J=3.0 Hz, 2×CHₐₓ), 1.17 (6H, d, J=6.25 Hz, 2×CH₃), 1.02 (2H, qd, J=12.9 Hz, J=3.1 Hz, 2×CHₐₓ

¹³C NMR (125 MHz, DMSO-d₆) δ_{C}: 171.3 (CO), 66.9 (CH—(CH₃)₂), 48.9 (CH—NH₃⁺), 40.6 (CH—CH₂—COOⁱPr), 33.2 (CH—CH₂COOⁱPr), 29.8 (CH₂), 29.7 (CH₂), 21.5 (CH₃);

HRMS: M⁺=186.14866 (delta=−1.1 ppm; C₁₀H₂₀O₂N). HR-ESI-MS-MS (CID=35%; rel. int. %): 169(100).

### Example 12

DI of Trans/Cis-4-Methylcyclohexane-1-Aminium Chloride (C·HCl+T·HCl (G=H)) with CvS_{W60C}-TA Covalently Immobilized on Porous Resin in Presence of Pyruvate in Continuous Flow Mode

The procedure was performed as presented in Example 10 modified in a way that that four serially connected SynBioCart columns filled with the covalently immobilized CvS_{W60C}-TA biocatalyst were applied for the dynamic isomerization of the cis/trans-diastereomeric mixture of 4-methylcyclohexan-1-aminium chloride (compounds C·HCl+T·HCl (G=H), 20 mM, cis:trans=42:58). The stationery operation of the reaction for 24 h afforded the trans-amine (compound T (G=H)) in deₜᵣₐₙₛ>99% (by GC) which was not isolated due to the volatility of the product.

### Example 13

DI of Trans/Cis-4-Ethylcyclohexane-1-Aminium Chloride (C·HCl+THC (G=Me)) with CvS_{W60C}-TA Covalently Immobilized on Porous Resin in Presence of Pyruvate in Continuous Flow Mode

The procedure was performed as presented in Example 10 modified in a way that that two serially connected SynBioCart columns filled with the covalently immobilized CvS_{W60C}-TA biocatalyst were applied for the dynamic isomerization of the cis/trans-diastereomeric mixture of 4-ethylcyclohexan-1-aminium chloride (compounds C·HCl+T·HCl (G=Me), 20 mM, cis:trans=65.4:34.6). The stationery operation of the reaction for 24 h (6-24 h) afforded the trans-amine hydrochloride salt product (compound C·HCl+T·HCl (G=Me), 10.7 mg, isolated yield 30.4%, deₜᵣₐₙₛ>99%) as a white solid.

¹H NMR (500 MHz, DMSO-d₆) δ_{H}: 7.99 (3H, br, NH₃⁺), 2.91-2.86 (1H, m CHₐₓ—NH₃⁺), 1.93-1.92 (2H, m, 2×CH_{eq}), 1.75-1.74 (2H, m, 2×CH_{eq}), 1.31-1.23 (2H, qd, J=12.6 Hz, J=3.25 Hz, 2×CHₐₓ), 1.21-1.16 (2H, quint, J=15 Hz CH₂CH₃), 1.07-1.03 (1H, m, CHₐₓ—CH₂CH₃), 0.94-0.88 (2H, qd, J=12.9 Hz, J=3.3 Hz, 2×CHₐₓ), 0.86-0.83 (3H, t, J=7.5 Hz, CH₂CH₃);

¹³C NMR (125 MHz, DMSO-d₆) δ_{C}: 49.42 (CH— NH₃⁺), 37.49 (CH—CH₂CH₃), 30.07 (CH₂), 29.92 (CH₂—CHCH₂CH₃), 28.69 (CH₂CH₃), 11.27 (CH₂CH₃);

HRMS: M+=128.14307 (delta=−2.4 ppm; C₈H₁₈N). HR-ESI-MS-MS (CID=35%; rel. int. %): 111(100) and 69(10).

### Example 14

DI of Trans/Cis-4-Phenylcyclohexane-1-Aminium Chloride (C·HCl+T·HCl (G=pH)) with CvS_{W60C}-TA Covalently Immobilized on Porous Resin in Presence of Pyruvate in Continuous Flow Mode

The procedure was performed as presented in Example 10 modified in a way that that two serially connected SynBioCart columns filled with the covalently immobilized CvS_{W60C}-TA biocatalyst were applied for the dynamic isomerization of the cis/trans-diastereomeric mixture of 4-phenylcyclohexan-1-aminium chloride (compounds C·HCl+T·HCl (G=Ph), 15 mM, cis:trans=26.2:73.8). The stationery operation of the reaction for 24 h (6-24 h) afforded the trans-amine hydrochloride salt product (compound C·HCl+T·HCl (G=Ph), (26.7 mg, isolated yield 70.7%) as a yellowish-white solid.

¹H NMR (500 MHz, DMSO-d₆) δ_{H}: 8.17 (3H, br, NH₃⁺), 7.29-7.26 (2H, m, ArHₘₑₜₐ), 7.24-7.22 (2H, m, ArHₒᵣₜₒ), 7.18 (1H, tt, J=7.11 Hz, J=1.43 Hz, ArHₚₐᵣₐ), 3.05-3.03 (1H, m, CHₐₓ—NH₃⁺), 2.46-2.40 (1H, m, CHₐₓ-Ph), 2.06-2.04 (2H, m, 2×CH_{eq}), 1.83-1.82 (2H, m, 2×CH_{eq}); 1.57-1.44 (4H, m, 4×CHₐₓ)

¹³C NMR (126 MHz, DMSO-d₆) δ_{C}: 146.0 (ArC), 128.2 (ArCHₘₑₜₐ), 126.6 (ArCHₒᵣₜₒ), 126.0 (ArCHₚₐᵣₐ), 48.8 (CH—NH₃⁺), 42.2 (CH-Ph), 31.4 (CH₂), 30.4 (CH₂);

HRMS: M⁺=176.14312 (delta=−1.5 ppm; C₁₂H₁₈N). HR-ESI-MS-MS (CID=35%; rel. int. %): 159(100); 91(3) and 81(3).

### Example 15

DI of Trans/Cis-4-Benzylcyclohexane-1-Aminium Chloride (C·HCl+T·HCl (G=CH₂pH)) with CvS_{W60C}-TA Covalently Immobilized on Porous Resin in Presence of Pyruvate in Continuous Flow Mode

The procedure was performed as presented in Example 10 modified in a way that that only one SynBioCart columns filled with the covalently immobilized CvS_{W60C}-TA biocatalyst was applied for the dynamic isomerization of the cis/trans-diastereomeric mixture of 4-benzylcyclohexan-1-aminium chloride (compounds C·HCl+T·HCl (G=CH₂Ph), 15 mM, cis:trans=50.7:49.3). The stationery operation of the reaction for 24 h (6-24 h) afforded the trans-amine hydrochloride salt product (compound C·HCl+T·HCl (G=CH₂Ph), (19.8 mg, isolated yield 54.1%) as a yellowish-white solid.

¹H NMR (500 MHz, DMSO-d₆) δ_{H}: 8.17 (3H, br, NH₃⁺), 7.29-7.26 (2H, m, ArHₘₑₜₐ), 7.24-7.22 (2H, m, ArHₒᵣₜₒ), 7.18 (1H, tt, J=7.11 Hz, J=1.43 Hz, ArHₚₐᵣₐ), 3.05-3.03 (1H, m, CHₐₓ—NH₃⁺), 2.46-2.40 (1H, m, CHₐₓ-Ph), 2.06-2.04 (2H, m, 2×CH_{eq}), 1.83-1.82 (2H, m, 2×CH_{eq}); 1.57-1.44 (4H, m, 4×CHₐₓ)

Dynamic Isomerization of Cis/Trans-Diastereomeric Mixtures of 4-Substituted Cyclohexane-1-Amines with ATA-217 (Engineered VfS-TA) in Batch Mode

### Example 16

DI of Trans/Cis-Methyl Esters (Ia+IIa) with Lyophilized ATA-217 in Presence of Pyruvate in Batch Mode

The procedure was performed as presented in Example 1 modified in a way that ATA-217 biocatalyst was used (at 1.0 mg/ml concentration) in the reaction starting from 25 mM of cis/trans diastereomeric mixture (IIa·HCl/Ia·HCl=48:52) in sodium phosphate buffer (100 mM, pH 7.5) supplemented with piridoxal-5-phosphate (PLP, 0.1 mM) and sodium pyruvate as amine acceptor (0.04 eq., 1 mM) the final reaction mixture (2 ml).

After 24 h reaction time, according to integration of peak areas for the ketone (IIa) and the corresponding acetamides of Ia and IIa, the molar fractions of the products Ia, IIa, and IIIa in the mixture were 76.9%, 19.2% and 3.9%, respectively; representing 24.7% cis to trans conversion and deₜᵣₐₙₛ=60.1% by GC.

### Example 17

DI of Trans/Cis-Ethyl Esters (Ib+IIb) with Lyophilized ATA-217 in Presence of Pyruvate in Batch Mode

The procedure was performed as presented in Example 1 modified in a way that ATA-217 biocatalyst was used (at 1.0 mg/ml concentration) in the reaction starting from 25 mM of cis/trans diastereomeric mixture (IIb·HCl/Ib·HCl=49:51) in sodium phosphate buffer (100 mM, pH 7.5) supplemented with piridoxal-5-phosphate (PLP, 0.1 mM) and sodium pyruvate as amine acceptor (0.04 eq., 1 mM) the final reaction mixture (2 ml).

After 24 h reaction time, according to integration of peak areas for the ketone (IIIb) and the corresponding acetamides of Ib and IIb, the molar fractions of the products Ib, IIb, and IIIb in the mixture were 84.6%, 11 3% and 4.0%, respectively; representing 34.0% cis to trans conversion and deₜᵣₐₙₛ=76.4% by GC.

### Example 18

DI of Cis-Ethyl Ester (IIb) with Lyophilized ATA-217 in Presence of Pyruvate in Batch Mode

The procedure was performed as presented in Example 1 modified in a way that ATA-217 biocatalyst was used (at 1.0 mg/ml concentration) in the reaction starting from 25 mM of cis diastereomer (IIb·HCl, de_{cis}=90.2%) in sodium phosphate buffer (100 mM, pH 7.5) supplemented with piridoxal-5-phosphate (PLP, 0.1 mM) and sodium pyruvate as amine acceptor (0.04 eq., 1 mM) the final reaction mixture (2 ml).

After 24 h reaction time, according to integration of peak areas for the ketone (IIIb) and the corresponding acetamides of Ib and IIb, the molar fractions of the products Ib, IIb, and IIIb in the mixture were 84.2%, 11.4% and 4.4%, respectively; representing 79.3% cis to trans conversion and deₜᵣₐₙₛ=76.0% by GC.

### Example 19

DI of Trans/Cis-Isopropyl Esters (Id+IId) with Lyophilized ATA-217 in Presence of Pyruvate in Batch Mode

The procedure was performed as presented in Example 1 modified in a way that ATA-217 biocatalyst was used (at 1.0 mg/ml concentration) in the reaction starting from 25 mM of cis/trans diastereomeric mixture (IId·HCl/Id·HCl=69:31) in sodium phosphate buffer (100 mM, pH 7.5) supplemented with piridoxal-5-phosphate (PLP, 0.1 mM) and sodium pyruvate as amine acceptor (0.04 eq., 1 mM) the final reaction mixture (2 ml).

After 24 h reaction time, according to integration of peak areas for the ketone (IIId) and the corresponding acetamides of Id and IId, the molar fractions of the products Id, IId, and IIId in the mixture were 84.4%, 10.9% and 4.7%, respectively; representing 53.4% cis to trans conversion and deₜᵣₐₙₛ=77.2% by GC.

### Example 20

Attempted Isomerization of Trans/Cis-2-(4-Aminocyclohexyl)Ethan-1-ol (IVa+Va) with Lyophilized ATA-217 in Presence of Pyruvate in Batch Mode

The procedure was performed as presented in Example 1 modified in a way that ATA-217 biocatalyst was used (at 1.0 mg/ml concentration) in the reaction starting from 25 mM of cis/trans diastereomeric mixture (Va/IVa=48.3:51.7) in sodium phosphate buffer (100 mM, pH 7.5) supplemented with piridoxal-5-phosphate (PLP, 0.1 mM) and sodium pyruvate as amine acceptor (0.04 eq., 1 mM) the final reaction mixture (2 ml).

After 24 h reaction time, according to integration of peak areas for the ketone (VIa) and the corresponding acetamides of IVa and Va, the molar fractions of the products IVa, Va, and VIa in the mixture were 49.8%, 31.0% and 19.2%, respectively; representing virtually no cis to trans conversion but deₜᵣₐₙₛ 23.2% by GC.

### Example 21

DI of Trans/Cis-4-(2-Acetoxyethyl)Cyclohexan-1-Amine [Compounds IV (R=Ac)+V (R=Ac)] with Lyophilized ATA-217 in Presence of Pyruvate in Batch Mode

The procedure was performed as presented in Example 1 modified in a way that ATA-217 biocatalyst was used (at 1.0 mg/ml concentration) in the reaction starting from 25 mM of cis/trans diastereomeric mixture of the O-acetate (V (R=Ac)·HCl/IV (R=Ac)·HCl=52.9:47.1) in sodium phosphate buffer (100 mM, pH 7.5) supplemented with piridoxal-5-phosphate (PLP, 0.1 mM) and sodium pyruvate as amine acceptor (0.04 eq., 1 mM) the final reaction mixture (2 ml).

After 24 h reaction time, according to integration of peak areas for the ketone [VI (R=Ac)] and the corresponding acetamides of IV (R=Ac) and V (R=Ac), the molar fractions of the products IV (R=Ac), V (R=Ac), and VI (R=Ac) in the mixture were 59.4%, 33.2% and 7.4%, respectively; representing 12.3% cis to trans conversion and deₜᵣₐₙₛ=28.3% by GC.

### Example 22

DI of Trans/Cis-4-((1,3-Dioxolan-2-Yl)Methyl)Cyclohexan-1-Amine (Compounds VIIa+VIIIa) with Lyophilized ATA-217 in Presence of Pyruvate in Batch Mode

The procedure was performed as presented in Example 1 modified in a way that ATA-217 biocatalyst was used (at 1.0 mg/ml concentration) in the reaction starting from 25 mM of cis/trans diastereomeric mixture of the O-acetate (VIIa/VIIIa=52.0:48.0) in sodium phosphate buffer (100 mM, pH 7.5) supplemented with piridoxal-5-phosphate (PLP, 0.1 mM) and sodium pyruvate as amine acceptor (0.04 eq., 1 mM) the final reaction mixture (2 ml).

After 24 h reaction time, according to integration of peak areas for the ketone (IXa) and the corresponding acetamides of VIIa and VIIIa, the molar fractions of the products VIIa, VIIIa, and IXa in the mixture were 74.9%, 11.7% and 13.4%, respectively; representing 22.9% cis to trans conversion and deₜᵣₐₙₛ=73.0% by GC.

### Example 23

DI of Trans/Cis-4-((1,3-Dioxan-2-Yl)Methyl)Cyclohexan-1-Amine [Compounds VII (n=2)+VIIIa (n=2)] with Lyophilized ATA-217 in Presence of Pyruvate in Batch Mode

The procedure was performed as presented in Example 1 modified in a way that ATA-217 biocatalyst was used (at 1.0 mg/ml concentration) in the reaction starting from 25 mM of cis/trans diastereomeric mixture of the O-acetate (VII (n=2)/VIII (n=2)=51.0:49.0) in sodium phosphate buffer (100 mM, pH 7.5) supplemented with piridoxal-5-phosphate (PLP, 0.1 mM) and sodium pyruvate as amine acceptor (0.04 eq., 1 mM) the final reaction mixture (2 ml).

After 24 h reaction time, according to integration of peak areas for the ketone [IXa (n=2)] and the corresponding acetamides of VII (n=2) and VIII (n=2), the molar fractions of the products VII (n=2), VIII (n=2), and IX (n=2) in the mixture were 60.9%, 27.3% and 11.7%, respectively; representing 9.9% cis to trans conversion and deₜᵣₐₙₛ=38.0% by GC.

1. A process of producing (1r,4r)-4-substituted cyclohexane-1-amine of formula (T) or a salt thereof 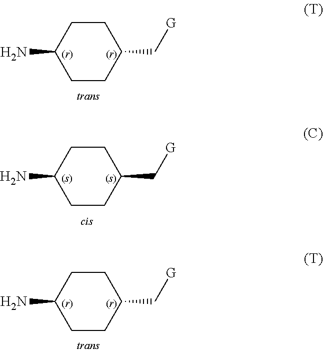
where in G represents a substituent, selected from
a hydrogen atom;
a C₁₋₆ alkyl group;
an ester moiety (—COOR), wherein R represents an alkyl, aralkyl, or aryl group;
a CH₂—OR′ group, wherein R′ represents a hydrogen atom or a hydroxyl protecting group;
a group of formula 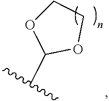
wherein n is an integer of 1 to 2;
a substituted or unsubstituted aryl group; or
an aralkyl group,
the process comprising:
reacting a diastereomeric mixture of 4-substituted cyclohexane-1-amines (formula (C)+formula (T)) or salts thereof 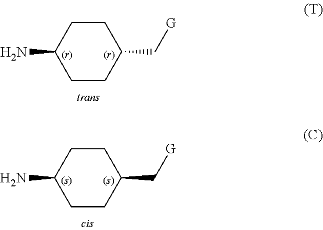
with a single transaminase biocatalyst in whole-cell, soluble, or immobilized form in the presence of an amine acceptor used in a sub-equimolar or equimolar quantity.

2. The process according to claim 1 characterized in that the reaction is carried out in batch mode or in continuous-flow mode.

3. (canceled)

4. The process according to claim 1 characterized in that the diastereomeric mixture of 4-substituted cyclohexane-1-amines (formula (C)+formula (T)) is in hydrochloride salt form (formula (C·HCl)+formula (T·HCl)).

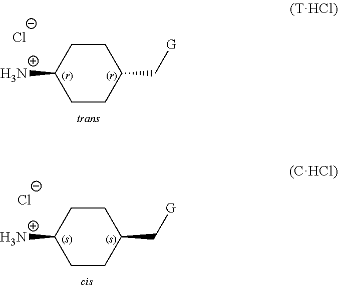

5. (canceled)

6. The process according to claim 1 characterized in that a transaminase biocatalyst comprising an amino acid sequence with at least about 37% sequence identity to Chromobacterium violaceum transaminase mutant (W60C) (CvS_{W60C}-TA: SEQ ID NO. 1) or to Vibrio fluvialis transaminase (VfS-TA: SEQ ID NO. 2) over a region of at least about 100 residues is used.

7.-11. (canceled)

12. The process according to claim 1 characterized in that a ketone or aldehyde is used as the amine acceptor in a sub-equimolar amount.

13. The process according to claim 1 characterized in that a 4-substituted cyclohexanone of formula K 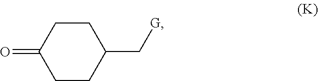
wherein G is as described in claim 1 for formula (C) and formula (T), is used as the amine acceptor-ketene.

14. The process according to claim 1 characterized in that the diastereomeric mixture consists of 2-(4-aminocyclohexyl)acetic acid esters of formula (I) and formula (II) or salts thereof 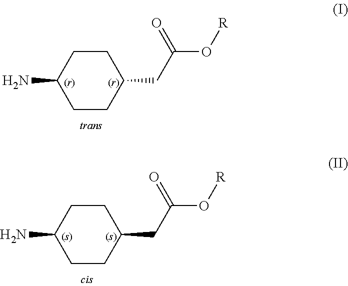
wherein R represents an alkyl, aralkyl, or aryl group.

15. The process according to claim 14 characterized in that sodium pyruvate is used as the amine acceptor in a sub-equimolar amount.

16. The process according to claim 14 characterized in that a 4-substituted cyclohexanone of formula (III) is used as the amine acceptor 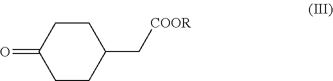
wherein R represents an alkyl, aralkyl, or aryl group.

17.-19. (canceled)

20. The process according to claim 14 characterized in that the transaminase biocatalyst is a Chromobacterium violaceum mutant (W60C) transaminase/CvS_{W60C}-TA, characterized by SEQ ID NO. 1/used in whole-cell form, immobilized whole-cell form, soluble cell-free form, or immobilized cell-free form, and wherein the transaminase biocatalyst is used in batch mode.

21. (canceled)

22. The process according to claim 14 characterized in that the transaminase biocatalyst is a Vibrio fluvialis transaminase/VfS-TA, characterized by SEQ ID NO. 2/used in whole-cell form, immobilized whole-cell form, soluble cell-free form, or immobilized cell-free form, and wherein the transaminase biocatalyst is used in batch mode.

23-24. (canceled)

25. The process according to claim 14 characterized in that the diastereomeric mixture consists of 2-(4-aminocyclohexyl)acetic acid ethyl ester hydrochloride salt (formula Ib·HCl+formula IIb·HCl) and pure 2-(trans-4-aminocyclohexyl)acetic ethyl ester (formula Ib) is produced.

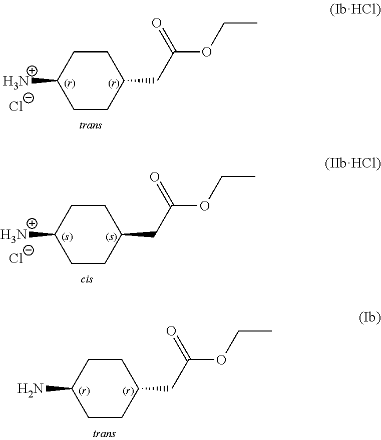

26. The process according to claim 14 characterized in that the diastereomeric mixture consists of 2-(4-aminocyclohexyl)acetic acid isopropyl ester hydrochloride salt (formula Id·HCl+formula IId·HCl) and pure 2-(trans-4-aminocyclohexyl)acetic isopropyl ester (formula Id) is produced.

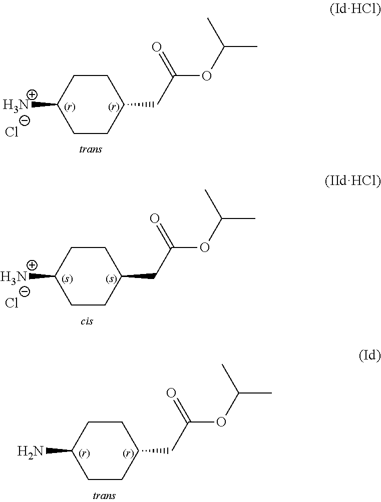

27. The process according to claim 1 characterized in that the diastereomeric mixture consists of 2-(4-aminocyclohexyl)ethan-1-ol derivatives of formula (IV) and formula (V) or salts thereof 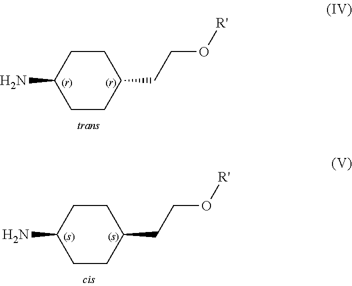
wherein R′ represents a hydrogen atom or hydroxyl-protecting group.

28. The process according to claim 27 characterized in that sodium pyruvate is used as the amine acceptor in a sub-equimolar amount.

29. The process according to claim 27 characterized in that a 4-substituted cyclohexanone of formula (VI) is used as the amine acceptor 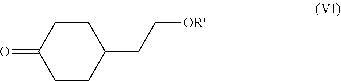
wherein R′ represents a hydrogen atom or hydroxyl-protecting group.

30. (canceled)

31. The process according to claim 27 characterized in that a Chromobacterium violaceum mutant (W60C) enzyme/CvS_{W60C}-TA, characterized by SEQ ID NO. 1/is used as the transaminase biocatalyst in batch mode.

32. (canceled)

33. The process according to claim 27 characterized in that a Vibrio fluvialis enzyme/VfS-TA, characterized by SEQ ID NO. 2/is used as the transaminase biocatalyst in batch mode.

34. (canceled)

35. The process according to claim 27 characterized in that a cis-selective Chromobacterium violaceum transaminase mutant (W60C)/CvS_{W60C}-TA/is used in continuous-flow mode.

36.-37. (canceled)

38. The process according to claim 1 characterized in that the starting diastereomeric mixture consists of 2-(4-aminocyclohexyl)acetaldehyde derivatives of formula (VII) and formula (VIII) or salts thereof 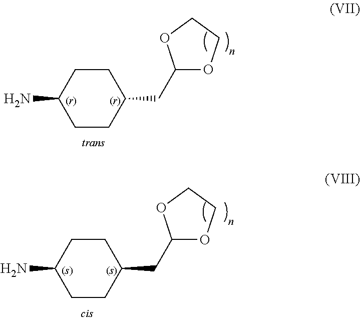
wherein n is an integer of 1 to 2.

39. (canceled)

40. The process according to claim 38 characterized in that a 4-substituted cyclohexanone of formula (IX) is used as the amine acceptor ketene 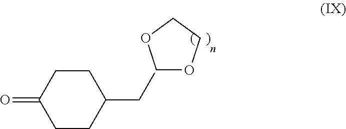
wherein n is an integer of 1 to 2.

41. The process according to claim 38 characterized in that the Chromobacterium violaceum mutant (W60C) enzyme/CvS_{W60C}-TA, characterized by SEQ ID NO. 1/is used as the transaminase biocatalyst in batch mode.

42. (canceled)

43. The process according to claim 38 characterized in that a Vibrio fluvialis enzyme/VfS-TA, characterized by SEQ ID NO. 2/is used as the transaminase biocatalyst in batch mode.

44. (canceled)

45. The process according to claim 38 characterized in that a cis-selective Chromobacterium violaceum transaminase mutant (W60C)/CvS_{W60C}-TA/is used in continuous-flow mode.

46.-47. (canceled)
